(19) United States
(12) Patent Application Publication
(10) Pub. No.: US 2024/0093034 A1
(43) Pub. Date: Mar. 21, 2024

(54) FLUORESCENT DYES

(71) Applicant: EXOPHARM LIMITED, Melbourne, Victoria (AU)

(72) Inventors: Jim PALMER, Melbourne (AU); Ian DIXON, Melbourne, Victoria (AU); Patrick JAMES, Melbourne, Victoria (AU); Melanie SCHOPPET, Melbourne, Victoria (AU); Karmen KONG, Melbourne, Victoria (AU); Sebastian M. MARCUCCIO, Scoresby, Victoria (AU); Jonathan M. FABER, Bayswater North, Victoria (AU); Kieran P. STOCKTON, Rowville, Victoria (AU); Melissa WERRETT, Prahran, Victoria (AU); Rohan JOYCE, Broadwater, Western Australia (AU)

(73) Assignee: EXOPHARM LIMITED, Melbourne, Victoria (AU)

(21) Appl. No.: 18/259,246

(22) PCT Filed: Dec. 24, 2021
(86) PCT No.: PCT/AU2021/051560

§ 371 Date: Jun. 23, 2023

(30) Foreign Application Priority Data
Dec. 24, 2020 (AU) 2020904852

(51) Int. Cl. C09B 55/00; G01N 33/58

(52) U.S. Cl. CPC C09B 55/009; G01N 33/582; G01N 2458/00

(57) ABSTRACT

The present invention relates to fluorescent dyes of formula (I), including salts thereof, and extracellular vesicles and cells labelled with the fluorescent dyes. The present invention also relates to methods, uses and kits thereof.

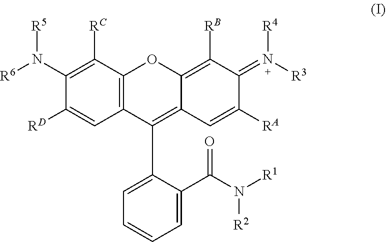

### FIELD OF THE INVENTION

The present invention relates to fluorescent dyes of formula (I), including salts thereof, and extracellular vesicles and cells labelled with the fluorescent dyes. The present invention also relates to methods of labelling extracellular vesicles and cells, and methods of preparing labelled extracellular vesicles and cells, using the fluorescent dye. The present invention further relates to uses and kits of the fluorescent dyes and salts of the invention.

### RELATED APPLICATION

This application claims priority from Australian provisional patent application AU 2020904852, the entire contents of which are hereby incorporated by reference.

### BACKGROUND OF THE INVENTION

Extracellular vesicles (EVs) are vesicles formed from the plasma membrane of cells. Unlike other vesicles such as liposomes, EVs possess characteristics of the cellular lipid bilayer, for example including the presence of extracellular proteins. EVs therefore are better able to evade the body's natural defence system and an immune response.

Several classes of EVs have been identified, including exosomes, microvesicles and apoptotic bodies. A variety of cell types can release EVs, including healthy and cancerous cells. While numerous functions of EVs have been postulated or established since their discovery, recent research has identified that EVs play a key role in intercellular communication based on their ability to transfer cargo such as nucleic acids (e.g. RNA) from cell to cell. There is currently significant interest in developing and using EVs as diagnostic markers of disease and for delivery of various therapeutic cargo. EVs derived from certain cell types, such as stem cells, are also of considerable interest themselves as potential therapeutics.

Given the potential diagnostic and therapeutic applications of EVs, there is interest in being able to detect and track EVs in order to gain further understanding of how they act. Preferably, methods for detecting and tracking EVs should be simple, inexpensive and use conventional instrumentation and techniques known in the field. While methods for detecting and/or tracking EVs have been developed, there is a continuing need for new methodology and systems for this purpose. Detecting and tracking EVs may be useful in further elucidating their biological function(s) and also potentially in their clinical use.

Accordingly, there is a need for new or alternative means that allow for detecting and/or tracking of EVs.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present inventors have identified fluorescent dyes which are capable of labelling EVs, which may advantageously allow EVs labelled with the dyes to be detected and tracked. The present inventors have identified that the fluorescent dyes are advantageously also capable of labelling cultured cells, including human cells.

Accordingly, in one aspect the present invention provides an extracellular vesicle (EV) labelled with a fluorescent dye of formula (I):

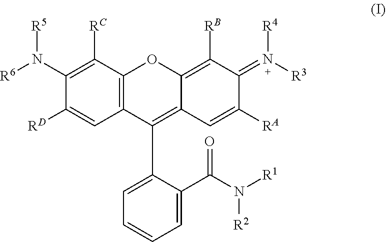

wherein
R¹ and R² are each independently selected from optionally substituted C₁₋₂₆aliphatic, optionally substituted C₁₋₂₆heteroaliphatic, optionally substituted C₁₋₂₆aliphatic-OH, optionally substituted C₁₋₂₆heteroaliphatic-OH and OH, wherein at least one of R¹ and R² is selected from optionally substituted C₁₃₋₂₆aliphatic or optionally substituted C₁₃₋₂₆heteroaliphatic;
R³, R⁴, R⁵ and R⁶ are independently selected from optionally substituted C₁₋₆alkyl, optionally substituted C₁₋₆haloalkyl, optionally substituted C₁₋₆heteroalkyl, optionally substituted aryl and optionally substituted C₁₋₆alkylaryl or
R³ and R⁴ together with the nitrogen atom to which they are attached form an optionally substituted 3-7 membered heterocyclic ring; and/or

R⁵ and R⁶ together with the nitrogen atom to which they are attached form an optionally substituted 3-7 membered heterocyclic ring; and

R^{A}, R^{B}, R^{C} and R^{D} are independently selected from hydrogen, fluorine and chlorine.

In another aspect, the present invention provides a salt of formula (II):

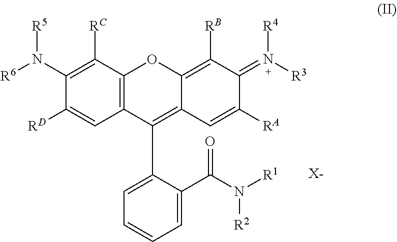

wherein
R¹ is selected from optionally substituted C₁₃₋₂₆aliphatic, C₁₃₋₂₆heteroaliphatic comprising 1 or 2 heteroatoms in the longest linear heteroaliphatic chain, optionally substituted C₁₃₋₂₆aliphatic-OH, optionally substituted C₁₃₋₂₆heteroaliphatic-OH and OH;
R² is selected from optionally substituted C₁₋₆aliphatic, optionally substituted C₁₋₆heteroaliphatic, optionally substituted C₁₋₆aliphatic-OH, optionally substituted C₁₋₆heteroaliphatic-OH, and OH;
R³, R⁴, R⁵ and R⁶ are independently selected from optionally substituted C₁₋₆alkyl, optionally substituted C₁₋₆haloalkyl, optionally substituted aryl and optionally substituted C₁₋₆alkylaryl or
R³ and R⁴ together with the nitrogen atom to which they are attached form an optionally substituted 3-7 membered heterocyclic ring; and/or
R³ and R⁴ together with the nitrogen atom to which they are attached form an optionally substituted 3-7 membered heterocyclic ring; and
R^{A}, R^{B}, R^{C} and R^{D} are independently selected from a hydrogen, fluorine and chlorine; and
X⁻ is selected from fluoride, chloride, bromide, iodide, acetate, trifluoroacetate, benzoate, C₁-C₆alkyl sulfonate, arylsulfonate, nitrate, hexafluorophosphate, tetrafluoroborate, trichlorozincate(II), tetrachloroferrate and perchlorate.

In another aspect, the present invention provides a salt of the compound of formula (I) and a counterion. In some embodiments, the counterion is selected from fluoride, chloride, bromide, iodide, acetate, trifluoroacetate, benzoate, C₁-C₆alkyl sulfonate, arylsulfonate, nitrate, hexafluorophosphate, tetrafluoroborate, trichlorozincate(II), tetrachloroferrate and perchlorate.

In another aspect, the present invention provides a method of labelling an extracellular vesicle (EV) or a cell, the method comprising combining the salt of formula (II) (e.g. the salt of a compound of formula (I)) described herein with the EV or the cell.

In another aspect, the present invention provides a method of preparing a labelled extracellular vesicle (EV), the method comprising combining the salt of formula (II) (e.g. the salt of a compound of formula (I)) described herein with the EV to thereby label the EV with the compound of formula (I).

In another aspect, the present invention provides a method of detecting an extracellular vesicle (EV) or a cell, the method comprising:
exposing an EV or cell labelled with a fluorescent dye of formula (I) described herein to a source of light selected to excite the fluorescent dye of formula (I); and
detecting fluorescence emission from the fluorescent dye.

In another aspect, the present invention provides a method of tracking an extracellular vesicle (EV) or a cell, the method comprising:
exposing an EV or cell labelled with a fluorescent dye of formula (I) defined herein to a source of light selected to excite the fluorescent dye of formula (I); and
detecting fluorescence emission from the fluorescent dye.

In another aspect, the present invention provides the use of the salt of the compound of formula (I) described herein for labelling an EV or a cell.

In another aspect, the present invention provides a kit for fluorescently labelling an EV or a cell, or when used for fluorescently labelling an EV or a cell, the kit comprising:
a salt of the compound of formula (I) described herein; and optionally instructions for its use to fluorescently label an extracellular vesicle or cell.
In another aspect, the present invention provides a method for delivering a therapeutic cargo to a cell, the method comprising contacting a cell with an EV labelled with a fluorescent dye of formula (I) as described herein, wherein the EV comprises a therapeutic cargo.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Flow cytometry image of a platelet-derived EV labelled with a fluorescent dye of the invention.

FIG. 2. Flow cytometry image of a normal human dermal fibroblasts (NHDF) cell labelled with a fluorescent dye of the invention.

FIG. 3. Microscopy images of (a) normal human dermal fibroblasts (NHDFs), (b) human umbilical cord-derived mesenchymal stem cells (UC-MSCs), and (c) bone marrow-derived MSCs (BM-MSCs) labelled with a fluorescent dye of the invention.

FIG. 4. Graphs showing fluorescence of (a) dyes 2 and 12-18 alone and (b) platelet-derived EV labelled with dyes 2 and 12-18 on NHDF cells. The fluorescence shown in (a) and (b) is normalised to dye 2 alone and EV labelled with dye 2, respectively.

FIG. 5. Graph showing fluorescence of a platelet-derived EV labelled with dye 2, and dyes 17 and 18, after passing through a ZebaTM Spin Desalting Column.

FIG. 6. Graph showing fluorescence of platelet-derived EV labelled with dyes 2 and 4, 6, 17, 18, 23, 24 and 25 on NHDF cells. The fluorescence is normalised to EV labelled with dye 2.

FIG. 7. Graphs showing delta cell index EVs labelled with dyes 2, 4, 6, 17,18, 23, 24 and 25 on proliferation of NHDF cells at (a) 24, (b) 36, (c) 48 and (d) 72 hours post-treatment, normalised to the basal media control for each time point. The samples are represented as follows: 0.1% basal media: filled circles; 2% complete media: filled squares; 10% high media: filled upward triangles; PBS control in 0.1% basal media: filled downward triangles; PLX+dye 23: filled diamonds; PLX+dye 24: unfilled circles; PLX+dye 25: unfilled squares; PLX+dye 18: unfilled upward triangles; PLX+dye 17: unfilled downward triangles; PLX+dye 4: unfilled diamonds; PLX+dye 6: asterisks; PLX+dye 2: stars; PLX alone: pluses. Significance was determined via Kruskal-Wallis nonparametric one-way analysis of variance, using multiple comparisons (uncorrected Dunn's test), where (*) denotes <0.05; (**) denotes <0.005; (***) denotes <0.0005, and non-significance is denoted by the lack of (*).

FIG. 8. Fluorescence labelling with conventional dyes and dye 2 of objects within an MSC-EV preparation. Fluorescent labelling procedures were performed for CFSE, Calcein AM, BODIPY, PKH67 and dye 2 in the absence of any EV preparation (upper row), and in the presence of MSC-EVs, alone (middle row) or in the presence of MSC-EVs and the detergent NP40. Fluorescence intensities of the dye labelled objects (x-axis) are plotted against the intensity of their size reflecting SSC signals (y-axis).

FIG. 9. (A) Gating strategy of the detected objects for MSC-EV preparations counterstained with PKH67 and anti-CD9 antibodies. Both fluorescence channels (CH02 and CH03) are initially plotted against the side scatter intensities (SSC) of all recorded objects. Number of co-incident objects per channel are depicted (2nd column). Of all recorded objects, the only objects considered in subsequent analyses were those that showed either single signals in the PKH67 or the antibody channel, or single signals in both channels (singlets). Within the Ch02 SSC singlet plots three different gates were defined with singlets in R1 and R2 with low and in R3 with concrete side scatter signals. Objects in R1 revealed no PKH67 and those in R2 and R3 concrete PKH67 signals. Ch02 signals plotted against SSC signals of the singlets are shown as well, either in the same plot size as in the left column before gating or in the zoom in versions of the same plots (right column). (B) Distribution of recorded singlets in R1, R2 and R3 without antibody labelling or following anti-CD9, anti-CD63 or anti-CD81 labelling, respectively. Plotting of the fluorescence intensities of singlets in the PKH67 (Ch02) or the antibody channel (Ch03) against the singlets' side scatter intensities. Column 3 to 5, fluorescence intensities of R1 to R3 gated singlets. (C) Number of events in gates R1-R3 for the respective measurements. The mean values ±standard deviation indicated.

FIG. 10. MSC-EV preparations counterstained with dye 2 and either anti-CD9, anti-CD63 or anti-CD81 antibodies. The same gating strategy as described in FIG. 9 was applied. Fluorescence intensities of singlets are plotted against the side scatter (SSC) intensities either for the antibody (Ch02) or the dye 2 (Ch03) channel. In the third column dye 2 signals are plotted against the signals of respective antibodies.

FIG. 11. Mixtures of PBMCs of 12 different donors were cultured in the presence or absence of non-labelled or dye 2-labelled MSC-EV preparations, or in the presence of the dye 2 for 5 days. Thereafter, cells were harvested and stained with DAPI and fluorescently labelled anti-CD4, anti-CD25 and anti-CD54 antibodies and analysed by conventional flow cytometry. (A) Gating strategy for CD4 T381 cells. Living cells were identified according to their forward and side scatter features as singlets and DAPI negative cells. CD4 T cells were gated as CD4⁺ living cells. (B) Fluorescent intensities of CD25 and CD54 gated living CD4⁺ cells of mdMLR assays cultured in the absence of any additives (stim), in the presence of non-labelled MSC-EVs (MSC-EVs), dye 2 labelled MSC-EVs (MSC-EVs+Dye 2) or in the presence of buffer solved dye 2 (Buffer+Dye 2).

FIG. 12. Analysis of the subcellular staining of immune cells of the mdMLR following uptake of dye 2-stained EVs via imaging flow cytometry. The light (bright field), fluorescent microscopy and merged images are shown.

FIG. 13. EVs stained with dye 2 of the invention in NHDF cells viewed by (A) conventional and (B) confocal microscopy. Platelet-derived EVs were stained with dye 2 (red). Nuclear stain was performed using Hoescht Stain (blue).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognise many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The present inventors have herein identified fluorescent dyes that are useful for labelling EVs and cells. The fluorescent dyes are analogues and derivatives of Rhodamine B, which is a fluorescent dye. The inventors have surprisingly found that the fluorescent dyes of the invention are capable of labelling EVs and cells, which may advantageously allow EVs and cells labelled with the dyes to be detected and tracked relatively easily and inexpensively using suitable commercially available instrumentation such as fluorimeters. Without wishing to be bound by theory, the present inventors hypothesise that the relatively hydrophobic moieties of R¹ and/or R² of the dyes of formula (I) may associate with the lipid bilayer of EVs and cells, which thereby allows them to incorporate into the EVs lipid bilayer to label the EV/cell with the dye. Also without wishing to be bound by theory, the inventors also believe that the relatively long chain lengths at R¹ and/or R² prevent dye aggregation and may allow for enhanced interaction of the positively charged rhodamine fluorophore to interact with negatively charges species on the surface of the EV or cell. It is expected that the dyes of the invention would also be capable of associating with, and therefore capable of labelling, other types of molecules or macromolecules that comprise a lipid bilayer.

As shown in the Examples, the fluorescent dyes of the invention have been shown to label EVs and various human cells. As also shown in the Examples, EVs and cells labelled with the fluorescent dyes of the invention are capable of being detected and imaged using conventional instrumentation.

It will be understood that the invention disclosed and defined in this specification extends to all alternative combinations of two or more of the individual features mentioned or evident from the text or drawings. All of these different combinations constitute various alternative aspects of the invention.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “and/or”, e.g., “X and/or Y” will be understood to mean either “X and Y” or “X or Y” and shall be taken to provide explicit support for both meanings or for either meaning.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±10%, ±5% or ±1% of that value.

As used herein, unless the context requires otherwise, the term “comprise”, and variations such as “comprises” and “comprising”, will be understood to imply the inclusion of a stated integer or step or group of integers or steps but not the exclusion of any other integer or step or group of integers or steps.

The term “aliphatic” by itself or as part of another substituent, means, unless otherwise stated, a straight or branched chain or combination thereof hydrocarbyl radical, which may be fully saturated, mono- or polyunsaturated and can include mono-, di- and multivalent radicals, having the number of carbon atoms designated (i.e. C₁-C₁₀ means one to ten carbons). Examples of saturated alkyl radicals include, but are not limited to, groups such as methyl, methylene, ethyl, ethylene, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1 -and 3-propynyl, 3-butynyl, and the higher homologs and isomers. The term “aliphatic” unless otherwise noted includes “alkyl”, “alkenyl” and “alkynyl”. In addition, references to the monovalent radical species of an aliphatic group, such as “alkyl,” unless otherwise noted, includes the multivalent radical species “alkylene”.

The term “heteroaliphatic” by itself or in combination with another term, means, unless otherwise stated, an aliphatic chain or combinations thereof interrupted by at least one heteroatom selected from the group consisting of O, N, Si and S (preferably O and N) that consists of the stated number of atoms (eg. a C₁-C₁₀heteroaliphatic means that the total number of carbon and hetero atoms numbers from 1 to 10 atoms). Any nitrogen and/or sulfur atoms included in a heteroaliphatic group may optionally be oxidized and a nitrogen heteroatom may optionally be quaternized. The heteroatom(s) O, N, S and Si may be at any valence allowed interior position of the heteroaliphatic group or the position at which the heteroaliphatic group is attached to the remainder of the molecule or to another group in the case of hybrid moieties (e.g. heteroaliphatic-OH). Examples include, but are not limited to, —CH₂—CH₂—O—CH₃, —CH₂—CH₂—NH—CH₃, —O—CH₂—CH₂—CH₃, —N H—CH₂—CH₂—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH=CH—O—CH₃, —Si(CH₃)₃, —CH₂—CH=N—OCH₃, and —CH=CH—N(CH₃)—CH₃. Up to two heteroatoms may be consecutive, such as, for example, —CH₂—NH—OCH₃ and —CH₂—O—Si(CH₃)₃. Preferably, the heteroaliphatic groups may comprise 1, 2, 3, 4 or more heteroatoms, and typically may not account for more than 40% of the atoms included in the longest linear heteroaliphatic chain. The “heteroaliphatic” group may be a “heteroalkyl”, “heteroalkenyl” or “heteroalkynyl” groups. In addition, a reference to the monovalent species includes a reference to the divalent radical, for example the term “heteroalkyl” includes “heteroalkylene”. Examples of heteroalkylene moieties include: —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like).

The term “alkyl” refers to a straight chain or branched saturated hydrocarbon group having 1 to 26 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl, decyl, undecyl, dodecyl, tricdecyl, tetradecyl, pentadecyl, hexadecyl (palmitadyl), heptadecanyl, octadecanyl (stearadyl), nonadecanyl, icosanyl, henicosanyl, docosanyl, tricosanyl, tetracosanyl, pentacosanyl and hexacosanyl.

The term “alkenyl” refers to a straight-chain or branched hydrocarbon group having one or more double bonds between carbon atoms and having 2 to 26 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl, undecenyl, dodecenyl, tridecenyl, tetradecenyl, pentadecenyl, hexadecenyl (palmitadenyl), heptadecenyl, octadecenyl (stearedyl), nonadecenyl, icosenyl, henicosenyl, docosenyl, tricosenyl, tetracosenyl, pentacosenyl and hexacosenyl.

The term “alkynyl” refers to a straight-chain or branched hydrocarbon group having one or more triple bonds between carbon atoms and having 2 to 26 carbon atoms. Where appropriate, the alkynyl group may have a specified number of carbon atoms. For example, C₂-C₆ as in “C₂-C₆alkynyl” includes groups having 2, 3, 4, 5 or 6 carbon atoms in a linear or branched arrangement. Examples of suitable alkynyl groups include, but are not limited to, ethynyl, propynyl, isopropynyl, butynyl, butadiynyl, pentynyl, pentadiynyl, hexynyl, hexadiynyl, heptynyl, octynyl, nonynyl, decynyl, undecynyl, dodecynyl, tridecynyl, tetradecynyl, pentadecynyl, hexadecynyl (palmitadynyl), heptadecynyl, octadecynyl (stearydyl), nonadecynyl, icosynyl, henicosynyl, docosynyl, tricosynyl, tetracosynyl, pentacosynyl and hexacosynyl.

The terms “cycloalkyl” and “heterocyclyl” by themselves or in combination with other terms, represent, unless otherwise stated, cyclic versions of “aliphatic” and “heteroaliphatic”, respectively. Also included are di- and multi-valent species such as “cycloalkylene.” Additionally, for heterocyclyl, a heteroatom (e.g. N or Si) can occupy the position at which the heterocycle is attached to the remainder of the molecule. Cycloalkyl and heterocyclyl groups may therefore be saturated or unsaturated. Unsaturated cycloalkyl and/or heterocyclyl groups may comprise 1 or 2 double bonds. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl”, by itself or in combination with other terms, refers to a carbocyclic (non-heterocyclic) aromatic ring or mono-, bi- or tri-cyclic ring system. Poly-cyclic ring systems may be referred to as “aryl” provided at least 1 of the rings within the system is aromatic. The aromatic ring or ring system is generally composed of 6 to 10 carbon atoms. Examples of aryl groups include, but are not limited to, phenyl, biphenyl, naphthyl and tetrahydronaphthyl. 6-membered aryls such as phenyl are preferred. The term “alkylaryl” refers to C₁₋₆alkylaryl such as benzyl.

The terms “halogen” and “halo”, by themselves or in combination with other terms, represent elements that constitute the Group Vila of the periodic table. Examples of halogens include, but are not limited to, fluorine, chlorine, bromine and iodine. The term “haloalkyl” refers to saturated alkyl radicals in which one or more hydrogens have been substituted with a halogen.

In the case of hybrid naming of substituent radicals describing two moieties that may both form a bond attaching the radical to the rest of the compound, such as alkylamino and alkylaryl, no direction in the order of groups is intended, so the point of attachment may be to any of the moieties included in the hybrid radical. For example, the terms “alkylaryl” and “arylalkyl”, are intended to refer to the same group and the point of attachment may be via the alkyl or the aryl moiety (or both in the case of diradical species). The direction of attachment of such a hybrid radical may be denoted by inclusion of a bond, for example, “-alkylaryl” or “arylalkyl-” denotes that the point of attachment of the radical to the rest of the compound is via the alkyl moiety, and “alkylaryl-” or “-arylalkyl” denotes that the point of attachment is via the aryl moiety.

### Fluorescent Dyes

The present invention relates to fluorescent dyes of formula (I):

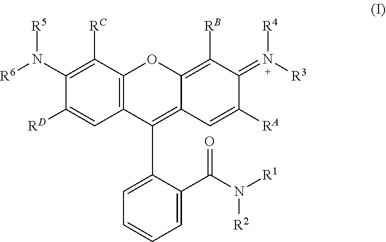

wherein
R¹ and R² are each independently selected from optionally substituted C₁₋₂₆aliphatic, optionally substituted C₁₋₂₆heteroaliphatic, optionally substituted C₁₋₂₆aliphatic-OH, optionally substituted C₁₋₂₆heteroaliphatic-OH and OH, wherein at least one of R¹ and R² is selected from optionally substituted C₁₃₋₂₆aliphatic or optionally substituted C₁₃₋₂₆heteroaliphatic;
R³, R⁴, R⁵ and R⁶ are independently selected from optionally substituted C₁₋₆alkyl, optionally substituted C₁₋₆haloalkyl, optionally substituted C₁₋₆heteroalkyl, optionally substituted aryl and optionally substituted C₁₋₆alkylaryl or
R³ and R⁴ together with the nitrogen atom to which they are attached form an optionally substituted 3-7 membered heterocyclic ring; and/or
R⁵ and R⁶ together with the nitrogen atom to which they are attached form an optionally substituted 3-7 membered heterocyclic ring; and
R^{A}, R^{B}, R^{C} and R^{D} are independently selected from hydrogen, fluorine and chlorine.

In any embodiment of formula (I), one or more R groups of formula (I) may be optionally substituted, that is, one or more R groups may be substituted or unsubstituted with one or more optional substituents.

Unless otherwise defined, the term “optionally substituted” or “optional substituent” as used herein refers to a group which may be unsubstituted or substituted further with 1, 2, 3, 4 or more groups, preferably 1, 2 or 3, more preferably 1 or 2 groups, selected from the group consisting of C₁₋₆alkyl, C₂₋₆alkenyl, C₂₋₆alkynyl, C₃-C₈cycloalkyl, hydroxyl, oxo, C₁₋₆alkoxy, aryloxy, C₁₋₆alkoxyaryl, halo, C₁₋₆alkylhalo (such as CF₃), C₁₋₆alkoxyhalo (such as OCF₃), carboxyl, esters, cyano, nitro, amino, substituted amino, disubstituted amino, acyl, ketones, substituted ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl.

For optionally substituted alkyl, alkenyl and alkynyl, the optional substituent or optional substituents are preferably selected from C₃₋₈cycloalkyl, hydroxyl, oxo, C₁₋₆alkoxy, aryloxy, C₁₋₆alkoxyaryl, halo, C₁₋₆alkylhalo (such as CF₃), C₁₋₆alkoxyhalo (such as OCF₃), carboxyl, esters, cyano, nitro, amino, substituted amino, disubstituted amino, acyl, ketones, substituted ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl, more preferably selected from halo, aryl, heterocyclyl, C₃₋₈cycloalkyl, C₁₋₆alkoxy, hydroxyl, oxo, aryloxy, haloC₁₋₆alkyl, haloC₁₋₆alkoxyl and carboxyl.

In some embodiments of formula (I):
R¹ is selected from C₁₃₋₂₆alkyl, C₁₃₋₂₆alkenyl, C₁₃₋₂₆heteroalkyl (preferably comprising 1 or 2 heteroatoms, preferably 1 or 2 oxygen atoms, in the longest linear heteroalkyl chain), and OH; preferably C₁₃₋₂₆alkyl, C₁₃₋₂₆alkenyl, and C₁₃₋₂₆heteroalkyl (preferably comprising 1 or 2 heteroatoms, preferably 1 or 2 oxygen atoms, in the longest linear heteroalkyl chain); more preferably C₁₃₋₂₆alkyl, C₁₃₋₂₆alkenyl, C₂alkyl-O-C₁₀₋₂₃a1kyl and OC₁₂₋₂₅alkyl, even more preferably C₁₄₋₂₂alkyl and C₁₄₋₂₆alkenyl; still more preferably C₁₄₋₂₂alkyl;
R² is selected from C₁₋₆alkyl, C₁₋₆heteroalkyl, C₁₋₆alkyl-OH and C₁₋₆heteroalkyl-OH; preferably C₁₋₆alkyl and C₁₋₆alkylOR⁸,wherein R⁸ is selected from hydrogen and methyl;
R³, R⁴, R⁵ and R⁶ are each independently a C₁₋₆alkyl or a C₁₋₆haloalkyl; preferably R³, R⁴, R⁵ and R⁶ are each independently a C₁₋₃alkyl or a C₁₋₃fluoroalkyl; more preferably R³, R⁴, R⁵ and R⁶ are each independently methyl, ethyl or CH₂CF3; even more preferably R³, R⁴, R⁵ and R⁶ are each ethyl;
R^{A}, R^{B}, R^{C} and R^{D} are each hydrogen.

The dye of formula (I) may be in the form of a salt. The salt comprises counterion X⁻ which is an anionic counter ion to achieve net electrical neutrality. The anionic counter ion X⁻ may be selected from inorganic and/or organic anions including, for example halides, carboxylates, carbonates, sulphates, sulfonates, sulphites, sulphides, nitrates, nitrites, phosphates, borates, alkoxides, and the like. Advantageously, salt forms of the compound of formula (I) may provide a convenient reagent for labelling an EV in any of the methods described herein.

Accordingly, the present invention provides a salt of a compound of formula (I). The salt may be a compound of formula (II):

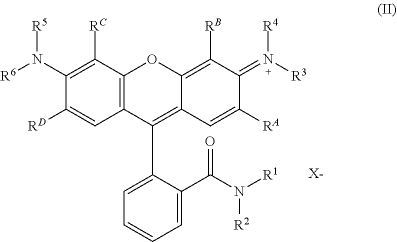

wherein
R¹ and R² are independently selected from optionally substituted C₁₋₂₆aliphatic, optionally substituted C₁₋₂₆heteroaliphatic, optionally substituted C₁₋₂₆aliphatic-OH, optionally substituted C₁₋₂₆heteroaliphatic-OH and OH, wherein at least one of R¹ and R² is selected from optionally substituted C₁₃₋₂₆aliphatic or optionally substituted C₁₃₋₂₆heteroaliphatic;
R³, R⁴, R⁵ and R⁶ are independently selected from optionally substituted C₁₋₆alkyl, optionally substituted C₁₋₆haloalkyl, optionally substituted aryl and optionally substituted C₁₋₆alkylaryl or
R³ and R⁴ together with the nitrogen atom to which they are attached form an optionally substituted 3-7 membered heterocyclic ring; and/or
R³ and R⁴ together with the nitrogen atom to which they are attached form an optionally substituted 3-7 membered heterocyclic ring; and
R^{A}, R^{B}, R^{C} and R^{D} are independently selected from a hydrogen, fluorine and chlorine; and
X⁻ is selected from fluoride, chloride, bromide, iodide, acetate, trifluoroacetate, benzoate, C₁₋₂₆alkyl sulfonate, arylsulfonate, nitrate, hexafluorophosphate, tetrafluoroborate, trichlorozincate(II), tetrachloroferrate and perchlorate.

In some embodiments of the salt of formula (II):
R¹ is selected from optionally substituted C₁₃₋₂₆aliphatic, C₁₃₋₂₆heteroaliphatic comprising 1 or 2 heteroatoms (preferably 1 or 2 oxygen atoms) in the longest linear heteroaliphatic chain, optionally substituted C₁₃₋₂₆aliphatic-OH, optionally substituted C₁₃₋₂₆heteroaliphatic-OH (preferably comprising 1 or 2 heteroatoms, preferably 1 or 2 oxygen atoms, in the longest linear heteroaliphatic chain excluding the terminal -OH oxygen atom), and OH;
R² is selected from optionally substituted C₁₋₆aliphatic, optionally substituted C₁₋₆heteroaliphatic, optionally substituted C₁₋₆aliphatic-OH, optionally substituted C₁₋₆heteroaliphatic-OH, and OH;
R³, R⁴, R⁵ and R⁶ are independently selected from optionally substituted C₁₋₆alkyl, optionally substituted C₁₋₆haloalkyl, optionally substituted aryl and optionally substituted C₁₋₆alkylaryl or
R³ and R⁴ together with the nitrogen atom to which they are attached form an optionally substituted 3-7 membered heterocyclic ring; and/or
R³ and R⁴ together with the nitrogen atom to which they are attached form an optionally substituted 3-7 membered heterocyclic ring; and
R^{A}, R^{B}, R^{C} and R^{D} are independently selected from a hydrogen, fluorine and chlorine; and
X⁻ is selected from fluoride, chloride, bromide, iodide, acetate, trifluoroacetate, benzoate, C₁₋₂₆alkyl sulfonate, arylsulfonate, nitrate, hexafluorophosphate, tetrafluoroborate, trichlorozincate(II), tetrachloroferrate and perchlorate.

In some embodiments of the salt of formula (II):
R¹ is selected from C₁₃₋₂₆alkyl, C₁₃₋₂₆alkenyl, C₁₃₋₂₆heteroalkyl comprising 1 or 2 heteroatoms, preferably 1 or 2 oxygen atoms, in the longest linear heteroalkyl chain, and OH; preferably C₁₃₋₂₆alkyl, C₁₃₋₂₆alkenyl, and C₁₃₋₂₆heteroalkyl comprising 1 or 2 heteroatoms (preferably 1 or 2 oxygen atoms) in the longest linear heteroalkyl chain; more preferably C₁₃₋₂₆alkyl, C₁₃₋₂₆alkenyl, C₂alkyl-O-C₁₀₋₂₃alkyl and OC₁₂₋₂₅alkyl; even more preferably C₁₄₋₂₂alkyl and C₁₄₋₂₂alkenyl; still more preferably C₁₄₋₂₂alkyl;
R² is selected from C₁₋₆alkyl, C₁₋₆heteroalkyl, C₁₋₆alkyl-OH and C₁₋₆heteroalkyl-OH; preferably C₁₋₆alkyl and C₁₋₆alkylOR⁸, wherein R⁸ is selected from hydrogen and methyl;
R³, R⁴, R⁵ and R⁶ are each independently a C₁₋₆alkyl or a C₁₋₆haloalkyl; preferably R³, R⁴, R⁵ and R⁶ are each independently a C₁₋₃alkyl or a C₁₋₃fluoroalkyl; more preferably R³, R⁴, R⁵ and R⁶ are each independently methyl, ethyl or CH₂CF3; even more preferably R³, R⁴, R⁵ and R⁶ are each ethyl;
R^{A}, R^{B}, R^{C} and R^{D} are each hydrogen; and
X⁻ is selected from chloride, bromide, iodide, acetate, trifluoroacetate, nitrate, hexafluorophosphate, tetrafluoroborate and perchlorate; preferably chloride, bromide and iodide; more preferably chloride.

In some embodiments of the salt of formula (II):
R¹ is selected from C₁₃₋₂₆alkyl, C₁₃₋₂₆alkenyl and C₁₃₋₂₆heteroalkyl comprising 1 or 2 heteroatoms, preferably 1 or 2 oxygen atoms, in the longest linear heteroalkyl chain; preferably C₁₃₋₂₆alkyl, C₁₃₋₂₆alkenyl, C₂alkyl-O-C₁₀₋₂₃alkyl and OC₁₂₋₂₅alkyl; more preferably C₁₄₋₂₂alkyl and C₁₄₋₂₂alkenyl; even more preferably C₁₄₋₂₂alkyl;
R² is selected from C₁₋₆alkyl, C₁₋₆heteroalkyl, C₁₋₆alkyl-OH and C₁₋₆heteroalkyl-OH; preferably C₁₋₆alkyl and C₁₋₆alkylOR⁸, wherein R⁸ is selected from hydrogen and methyl;
R³, R⁴, R⁵ and R⁶ are each independently a C₁₋₆alkyl or a C₁₋₆haloalkyl; preferably R³, R⁴, R⁵ and R⁶ are each independently a C₁₋₃alkyl or a C₁₋₃fluoroalkyl; more preferably R³, R⁴, R⁵ and R⁶ are each independently methyl, ethyl or CH₂CF₃; even more preferably R³, R⁴, R⁵ and R⁶ are each ethyl;
R^{A}, R^{B}, R^{C} and R^{D} are each hydrogen; and
X⁻ is selected from chloride, bromide, iodide, acetate, trifluoroacetate, nitrate, hexafluorophosphate, tetrafluoroborate and perchlorate; preferably chloride, bromide and iodide; more preferably chloride.

In some embodiments, the salt of formula (II) has a structure selected from any one of the following:

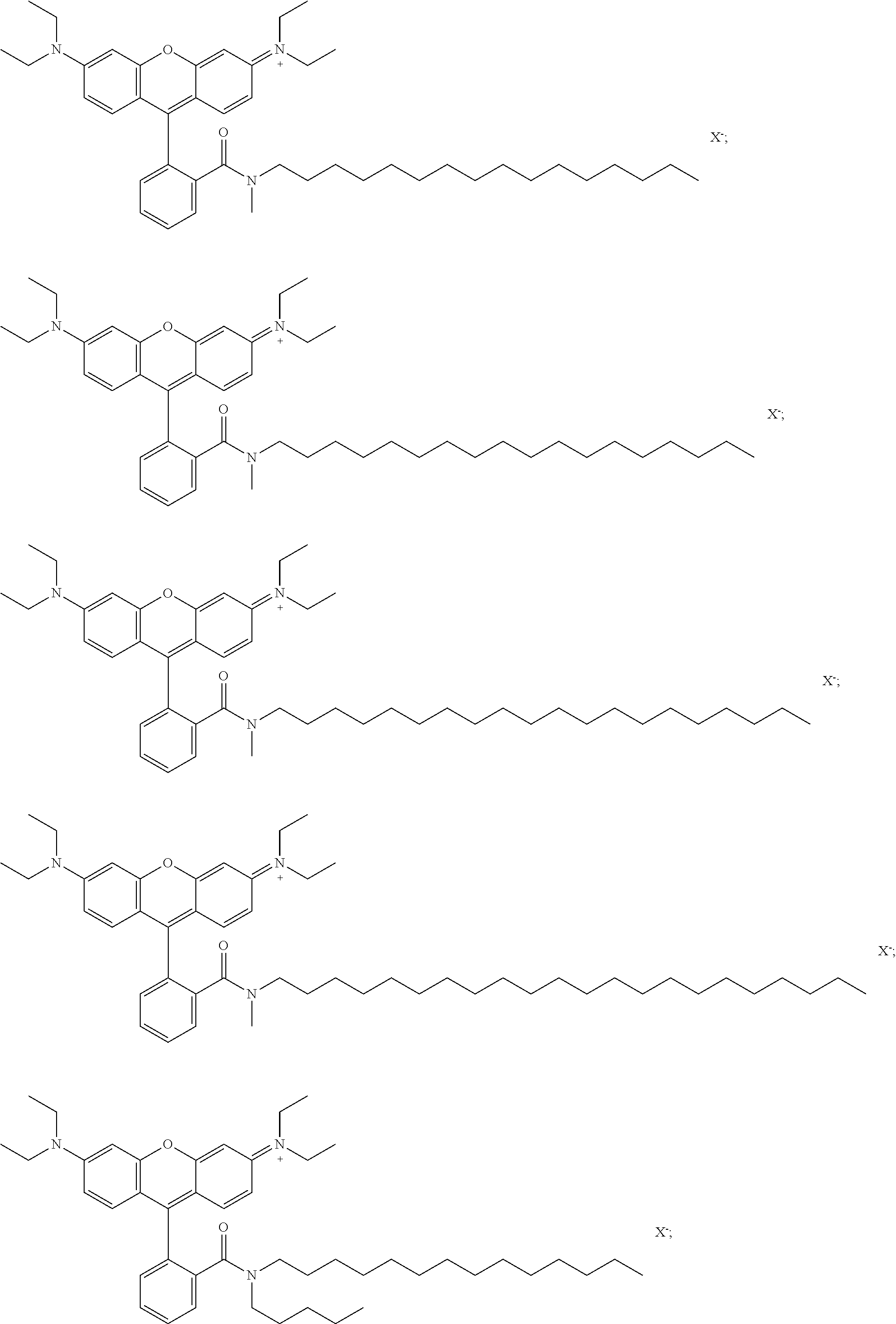

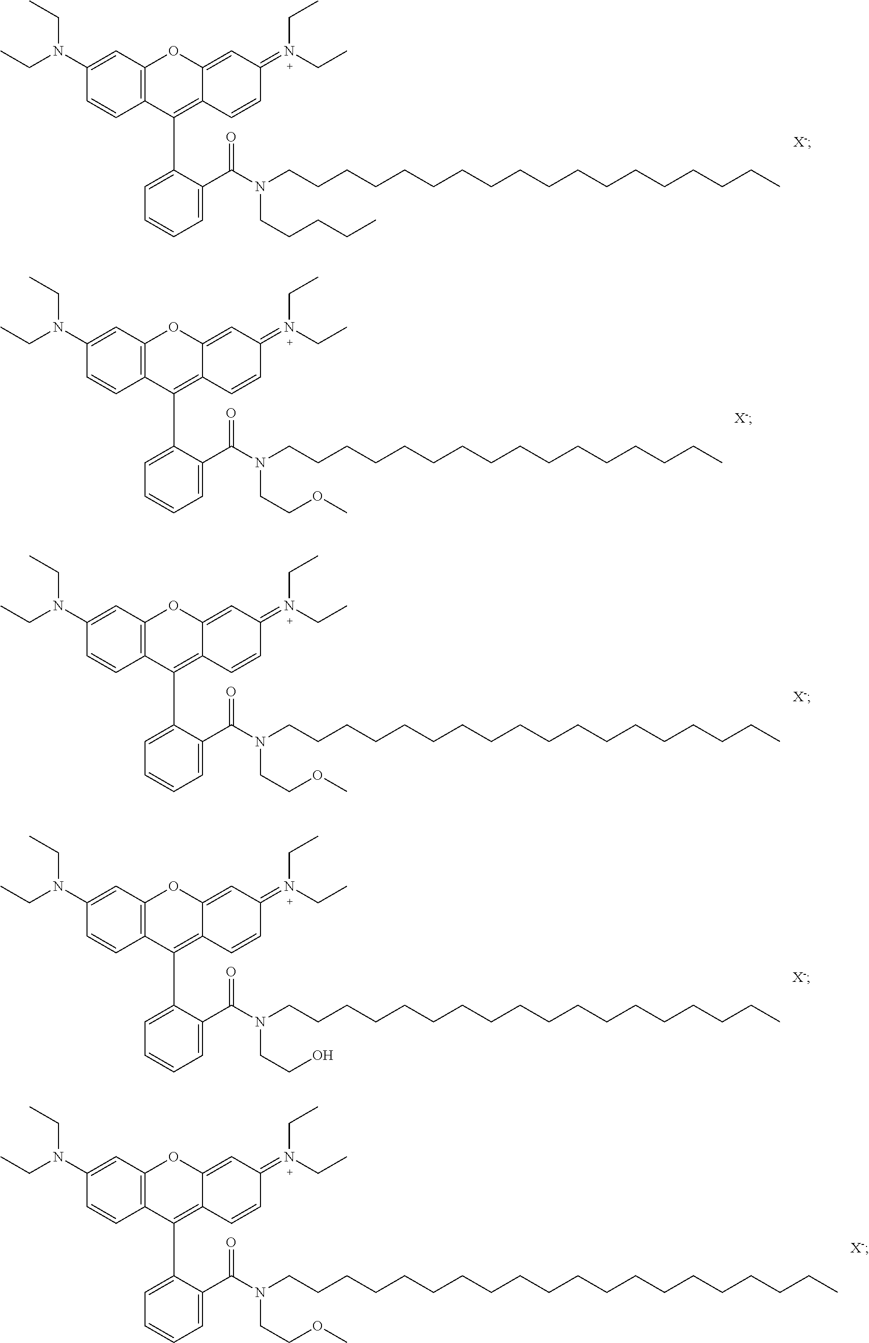

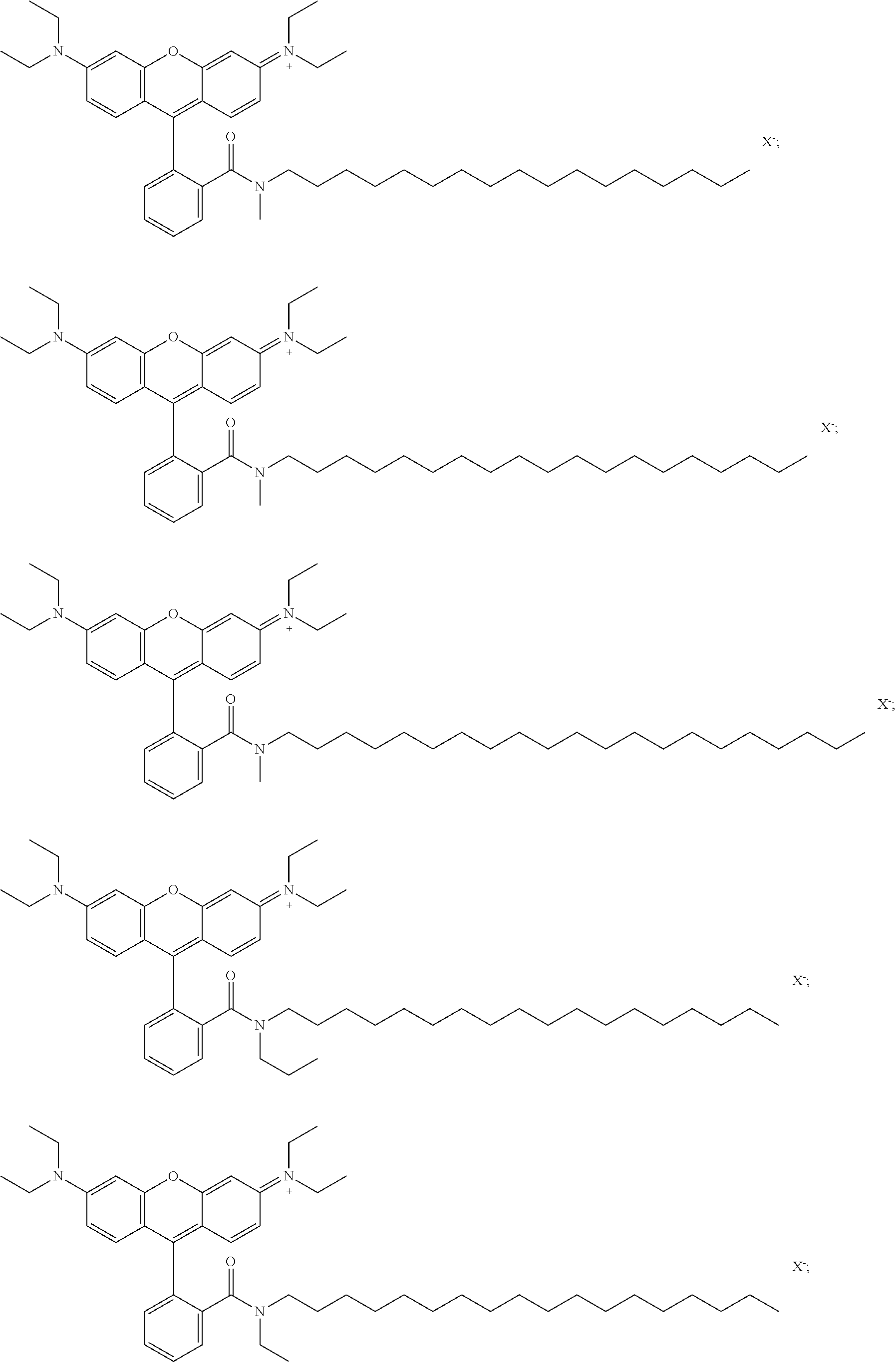

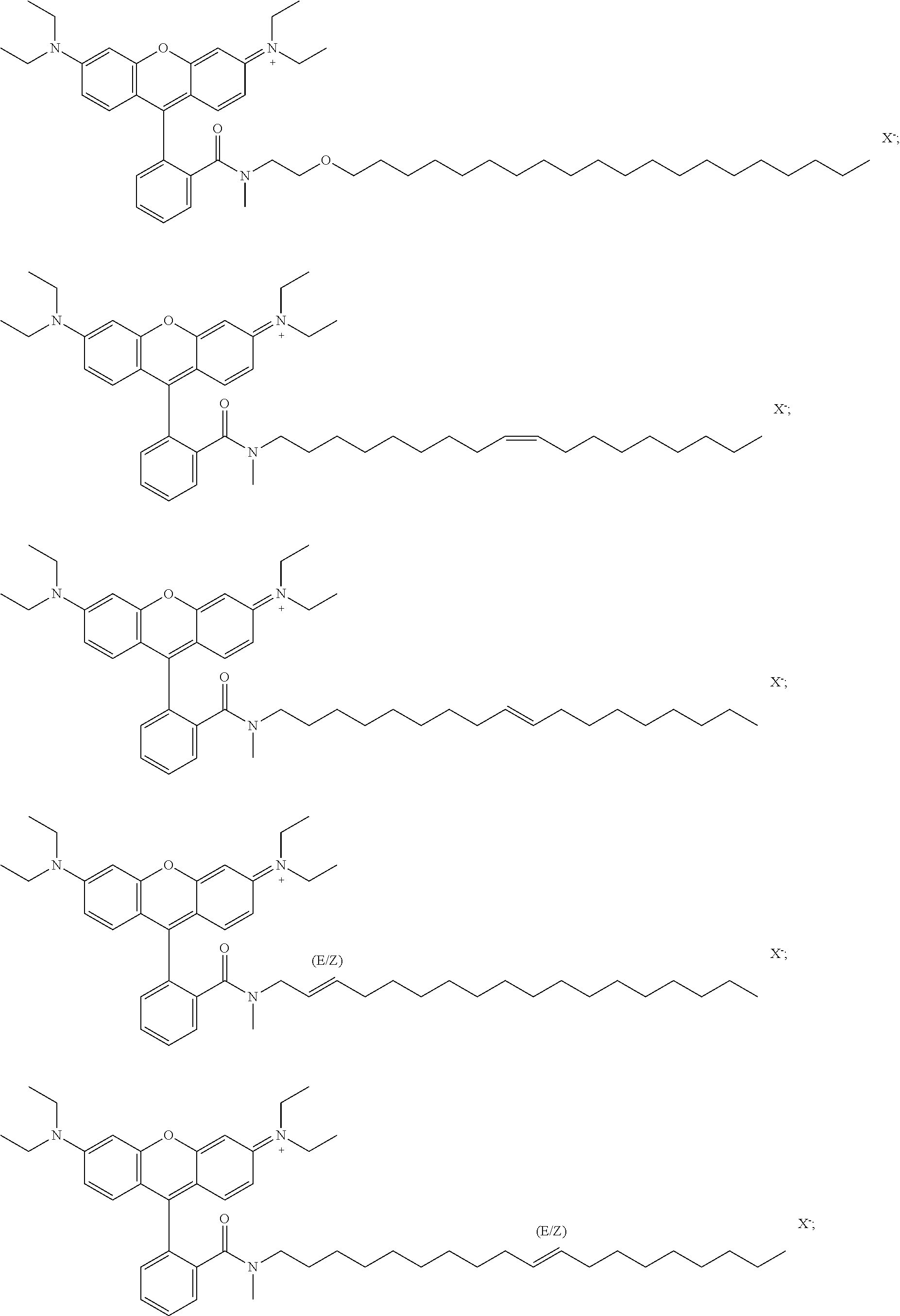

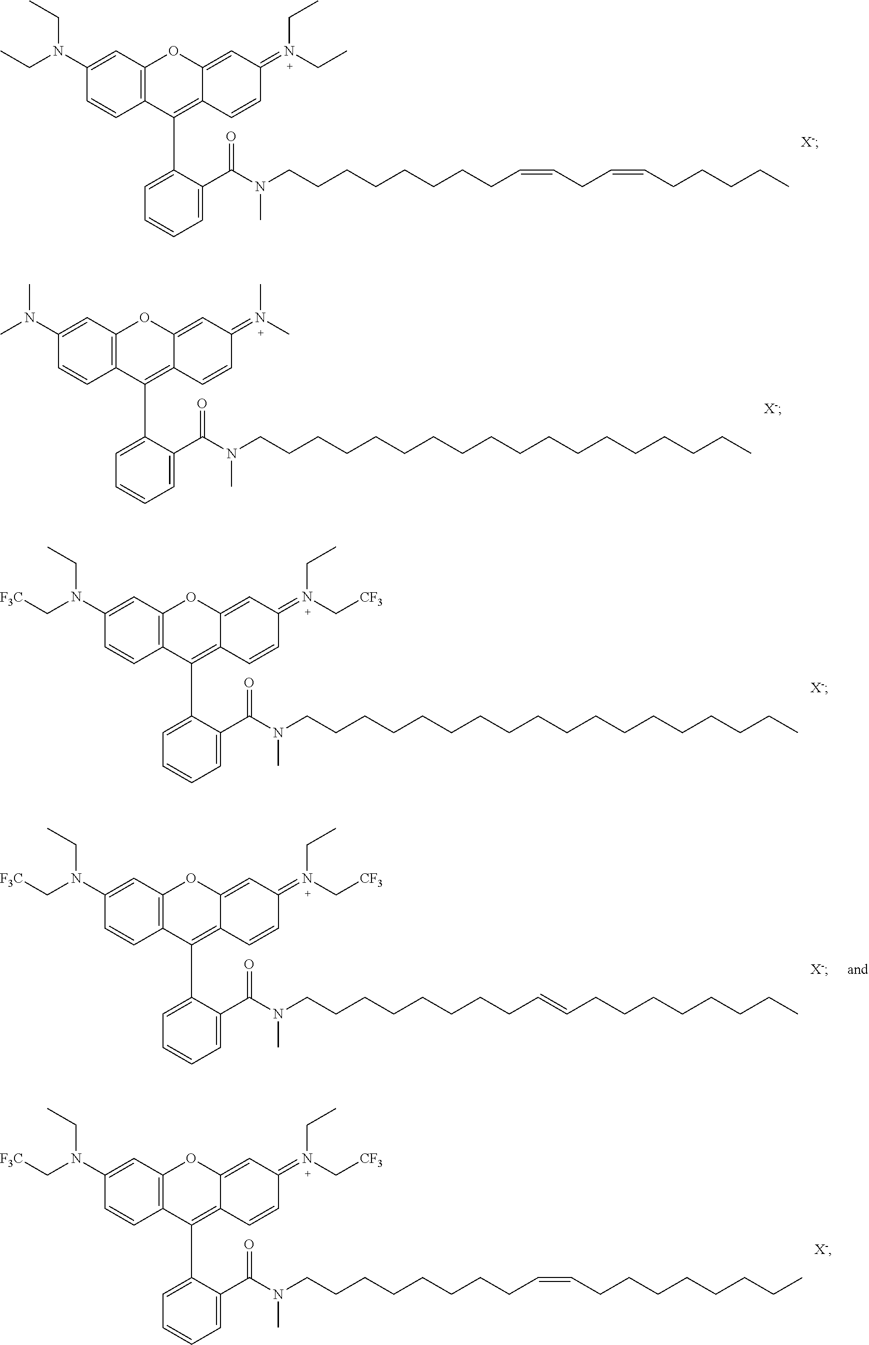

wherein X⁻ is selected from chloride, bromide, iodide, acetate, trifluoroacetate, nitrate, hexafluorophosphate, tetrafluoroborate and perchlorate; preferably chloride, bromide and iodide; more preferably chloride.

In some embodiments, the salt of formula (II) has a structure selected from any one of the following:

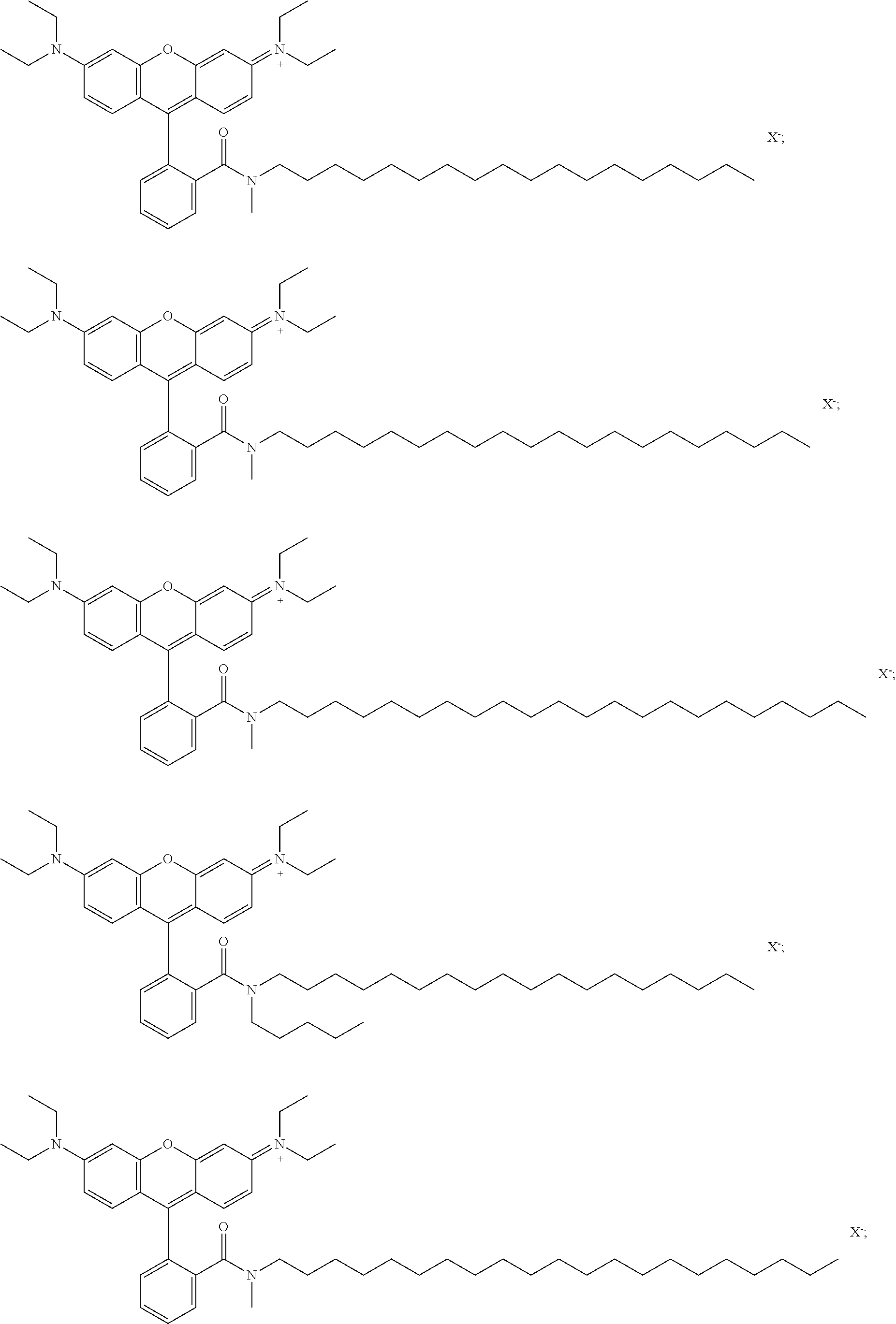

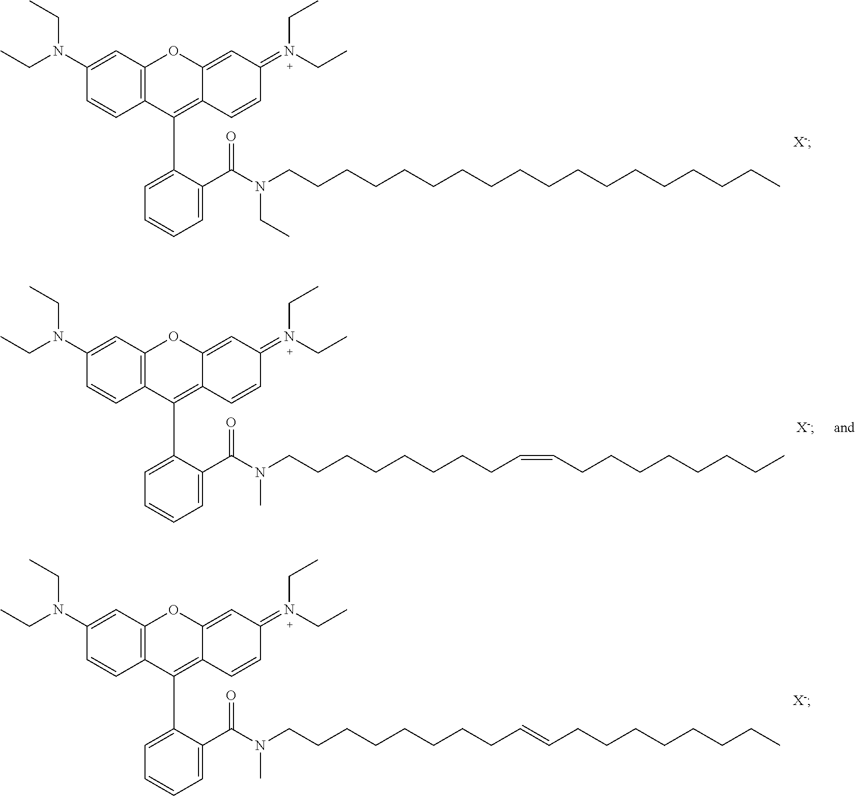

wherein X⁻ is selected from chloride, bromide, iodide, acetate, trifluoroacetate, nitrate, hexafluorophosphate, tetrafluoroborate and perchlorate; preferably chloride, bromide and iodide; more preferably chloride.

In some embodiments, the salt of formula (II) has a structure selected from any one of the following:

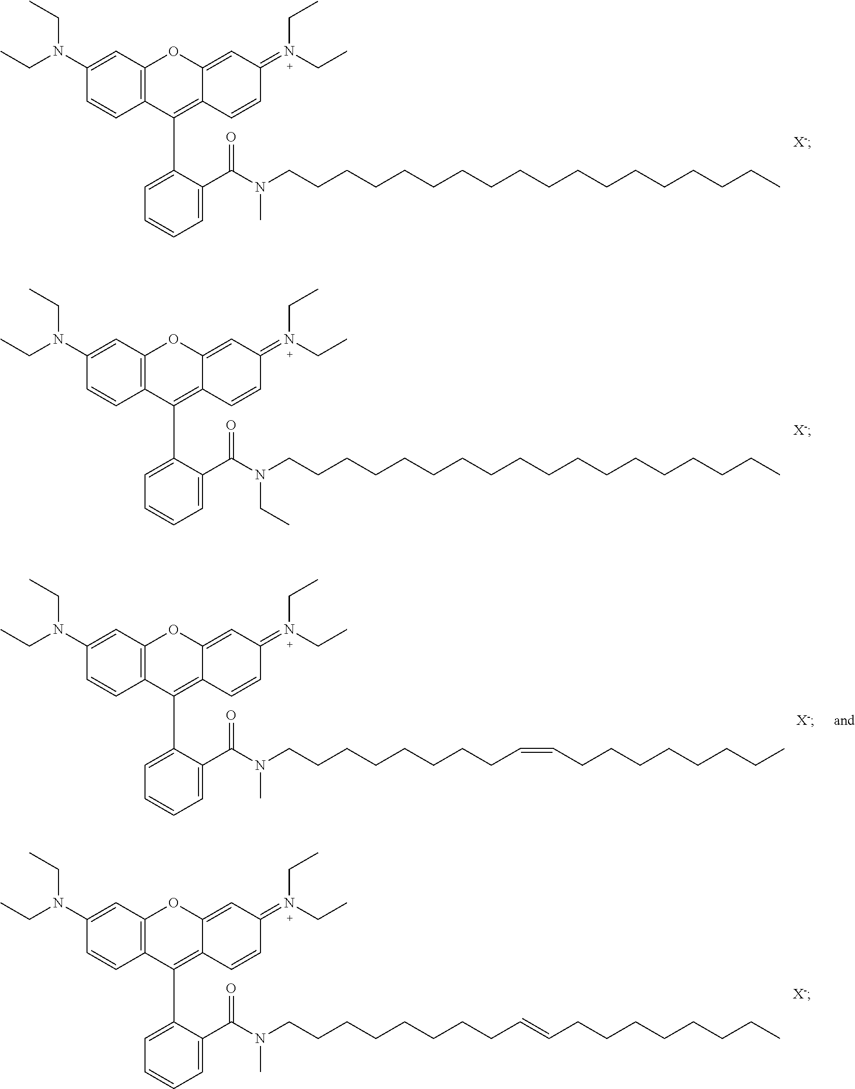

wherein X⁻ is selected from chloride, bromide, iodide, acetate, trifluoroacetate, nitrate, hexafluorophosphate, tetrafluoroborate and perchlorate; preferably chloride, bromide and iodide; more preferably chloride.

In one embodiment, the salt of formula (II) has the following structure:

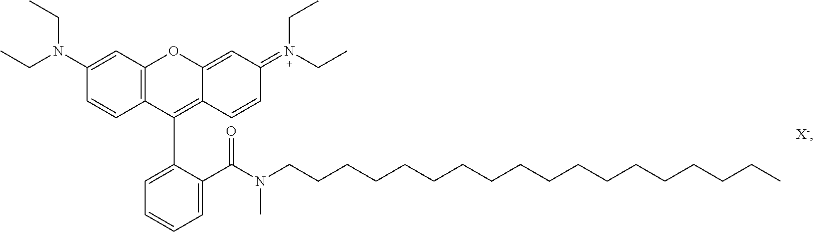

wherein X⁻ is selected from chloride, bromide, iodide, acetate, trifluoroacetate, nitrate, hexafluorophosphate, tetrafluoroborate and perchlorate; preferably chloride, bromide and iodide; more preferably chloride.

In some embodiments, the salt of formula (II) is selected from any one of the following:

| | Structure |
|---|---|
| 1 | 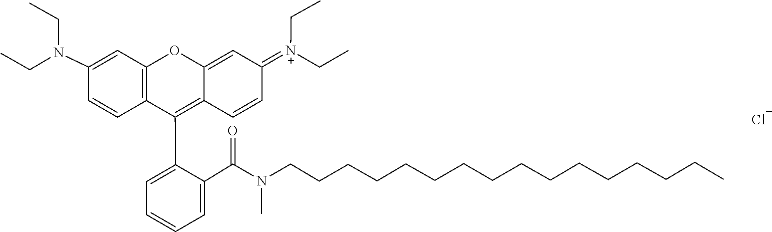 [SMILES: CCCCCCCCCCCCCCCCN(C)C(=O)c1ccccc1-c1c2ccc(=[N+](CC)CC)cc-2oc2cc(N(CC)CC)ccc12 ; [Cl-]] |
| 2 | 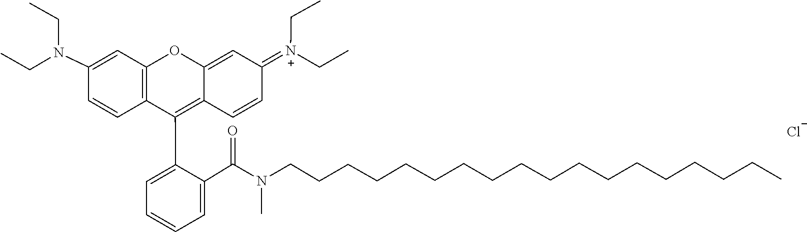 [SMILES: CCCCCCCCCCCCCCCCCCN(C)C(=O)c1ccccc1-c1c2ccc(=[N+](CC)CC)cc-2oc2cc(N(CC)CC)ccc12 ; [Cl-]] |
| 3 | 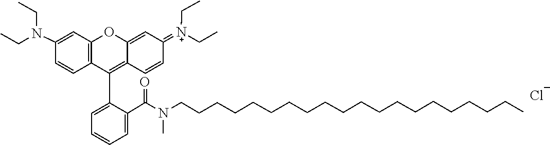 [SMILES: CCCCCCCCCCCCCCCCCCCCN(C)C(=O)c1ccccc1-c1c2ccc(=[N+](CC)CC)cc-2oc2cc(N(CC)CC)ccc12 ; [Cl-]] |
| 4 | 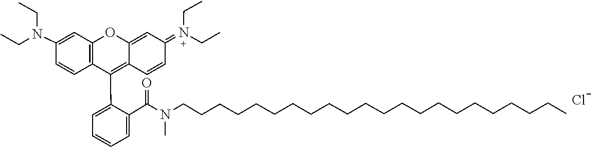 [SMILES: CCCCCCCCCCCCCCCCCCCCCCN(C)C(=O)c1ccccc1-c1c2ccc(=[N+](CC)CC)cc-2oc2cc(N(CC)CC)ccc12 ; [Cl-]] |
| 5 | 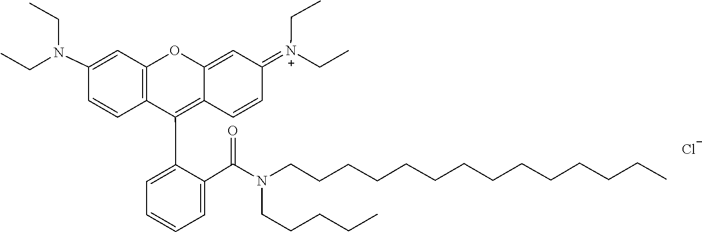 [SMILES: CCCCCCCCCCCCCCN(CCCCC)C(=O)c1ccccc1-c1c2ccc(=[N+](CC)CC)cc-2oc2cc(N(CC)CC)ccc12 ; [Cl-]] |
| 6 | 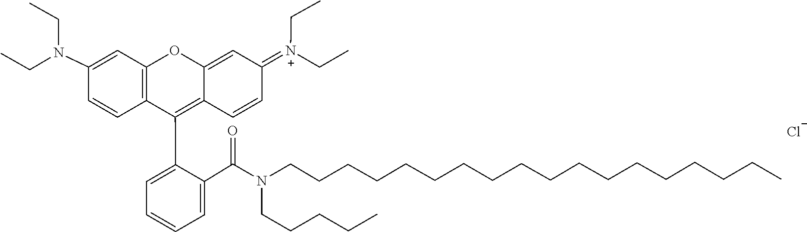 [SMILES: CCCCCCCCCCCCCCCCCCN(CCCCC)C(=O)c1ccccc1-c1c2ccc(=[N+](CC)CC)cc-2oc2cc(N(CC)CC)ccc12 ; [Cl-]] |
| 7 | 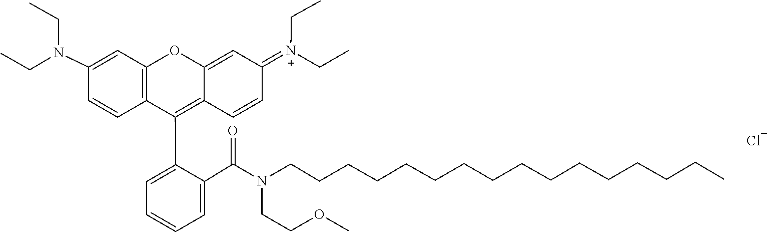 [SMILES: CCCCCCCCCCCCCCCCN(CCOC)C(=O)c1ccccc1-c1c2ccc(=[N+](CC)CC)cc-2oc2cc(N(CC)CC)ccc12 ; [Cl-]] |
| 8 | 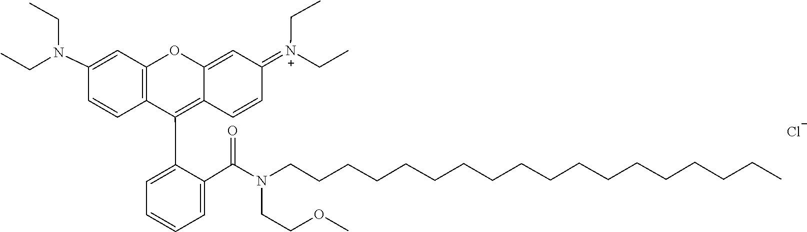 [SMILES: CCCCCCCCCCCCCCCCCCN(CCOC)C(=O)c1ccccc1-c1c2ccc(=[N+](CC)CC)cc-2oc2cc(N(CC)CC)ccc12 ; [Cl-]] |
| 9 | 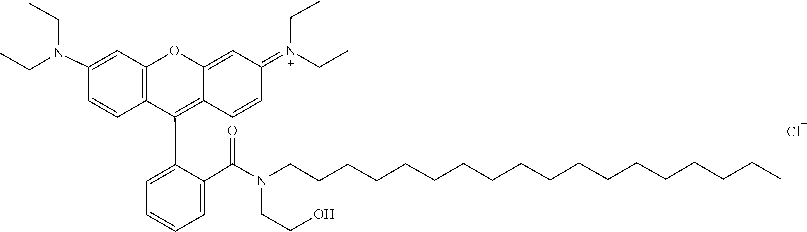 [SMILES: CCCCCCCCCCCCCCCCCCN(CCO)C(=O)c1ccccc1-c1c2ccc(=[N+](CC)CC)cc-2oc2cc(N(CC)CC)ccc12 ; [Cl-]] |
| 10 | 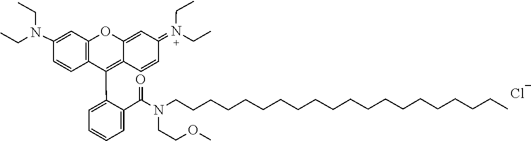 [SMILES: CCCCCCCCCCCCCCCCCCCCN(CCOC)C(=O)c1ccccc1-c1c2ccc(=[N+](CC)CC)cc-2oc2cc(N(CC)CC)ccc12 ; [Cl-]] |
| 11 | 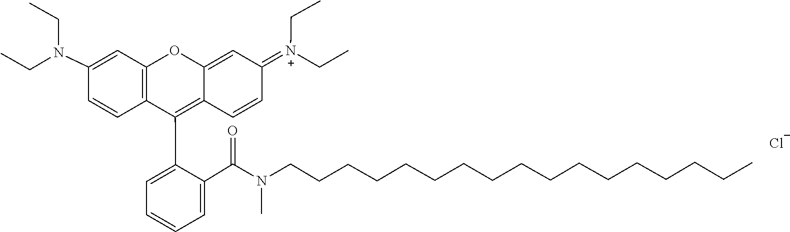 [SMILES: CCCCCCCCCCCCCCCCCN(C)C(=O)c1ccccc1-c1c2ccc(=[N+](CC)CC)cc-2oc2cc(N(CC)CC)ccc12 ; [Cl-]] |
| 12 | 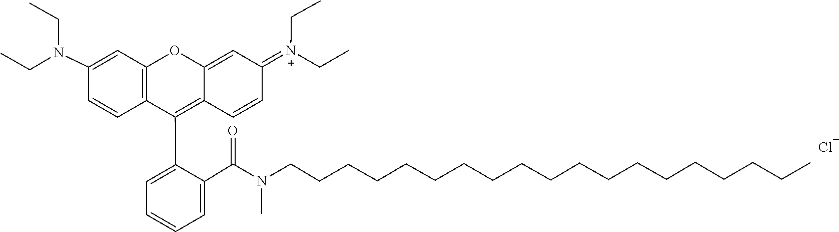 [SMILES: CCCCCCCCCCCCCCCCCCCN(C)C(=O)c1ccccc1-c1c2ccc(=[N+](CC)CC)cc-2oc2cc(N(CC)CC)ccc12 ; [Cl-]] |
| 13 | 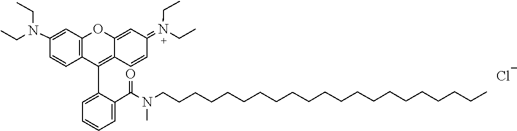 |
| 14 | 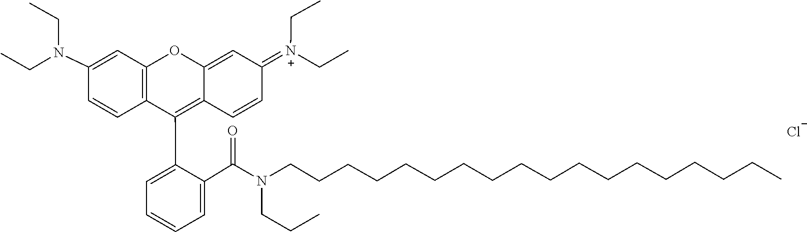 [SMILES: CCCCCCCCCCCCCCCCCCN(CCC)C(=O)c1ccccc1-c1c2ccc(=[N+](CC)CC)cc-2oc2cc(N(CC)CC)ccc12 ; [Cl-]] |
| 15 | 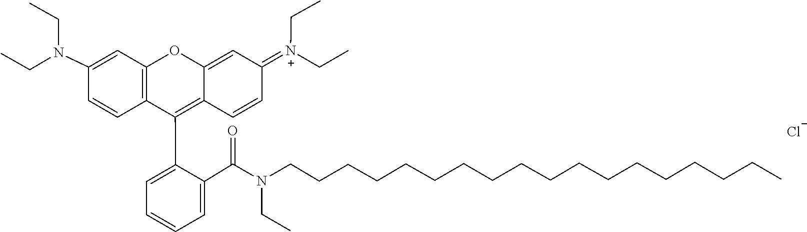 [SMILES: CCCCCCCCCCCCCCCCCCN(CC)C(=O)c1ccccc1-c1c2ccc(=[N+](CC)CC)cc-2oc2cc(N(CC)CC)ccc12 ; [Cl-]] |
| 16 | 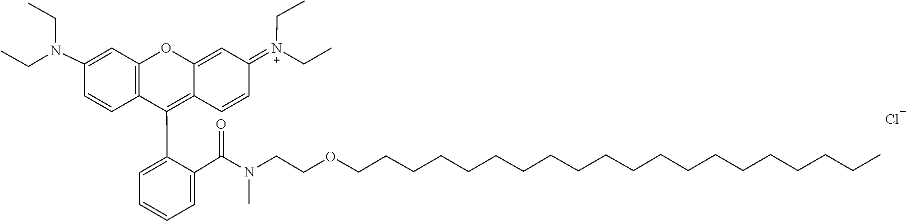 [SMILES: CCCCCCCCCCCCCCCCCCCCOCCN(C)C(=O)c1ccccc1-c1c2ccc(=[N+](CC)CC)cc-2oc2cc(N(CC)CC)ccc12 ; [Cl-]] |
| 17 | 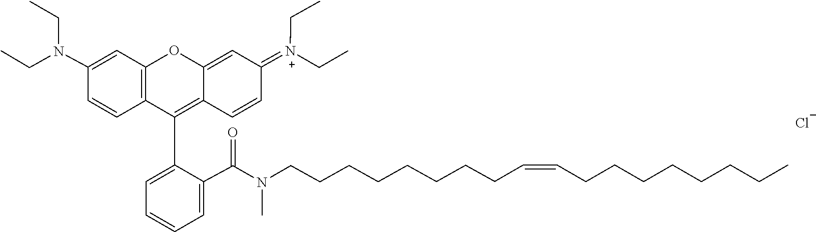 [SMILES: CCCCCCCC/C=C\CCCCCCCCN(C)C(=O)c1ccccc1-c1c2ccc(=[N+](CC)CC)cc-2oc2cc(N(CC)CC)ccc12 ; [Cl-]] |
| 18 | 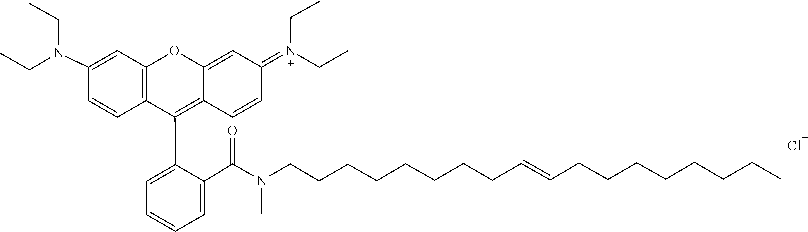 [SMILES: CCCCCCCC/C=C/CCCCCCCCN(C)C(=O)c1ccccc1-c1c2ccc(=[N+](CC)CC)cc-2oc2cc(N(CC)CC)ccc12 ; [Cl-]] |
| 19 | 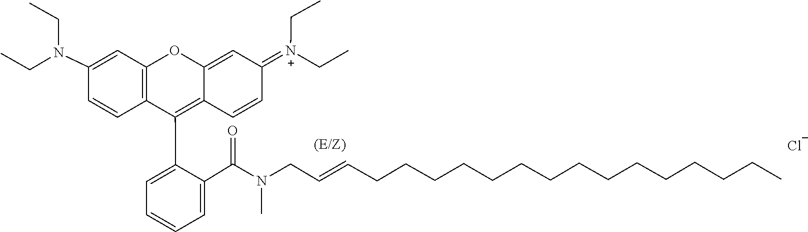 [SMILES: CCCCCCCCCCCCCCC/C=C/CN(C)C(=O)c1ccccc1-c1c2ccc(=[N+](CC)CC)cc-2oc2cc(N(CC)CC)ccc12 ; [Cl-]] |
| 20 | 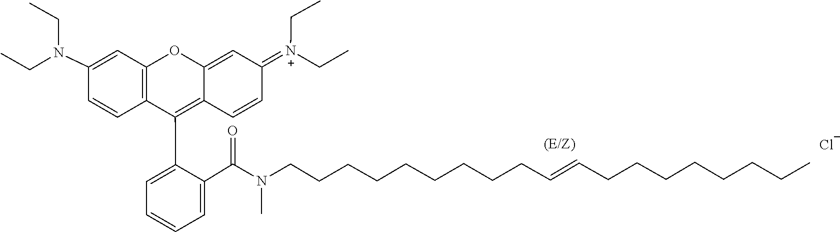 [SMILES: CCCCCCCC/C=C/CCCCCCCCCN(C)C(=O)c1ccccc1-c1c2ccc(=[N+](CC)CC)cc-2oc2cc(N(CC)CC)ccc12 ; [Cl-]] |
| 21 | 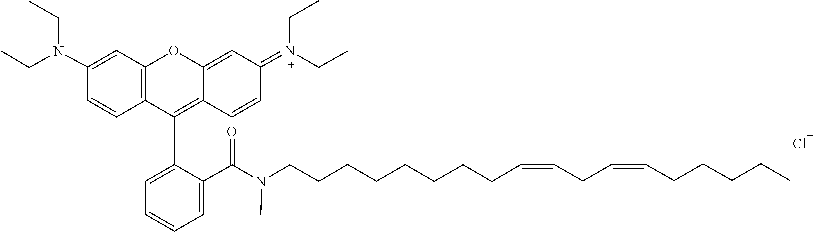 [SMILES: CCCCC/C=C\C/C=C\CCCCCCCCN(C)C(=O)c1ccccc1-c1c2ccc(=[N+](CC)CC)cc-2oc2cc(N(CC)CC)ccc12 ; [Cl-]] |
| 22 | 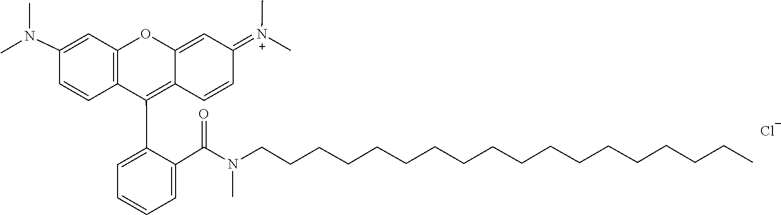 [SMILES: CCCCCCCCCCCCCCCCCCN(C)C(=O)c1ccccc1-c1c2ccc(=[N+](C)C)cc-2oc2cc(N(C)C)ccc12 ; [Cl-]] |
| 23 | 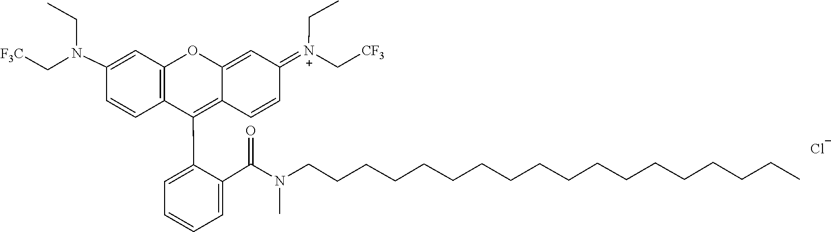 [SMILES: CCCCCCCCCCCCCCCCCCN(C)C(=O)c1ccccc1-c1c2cc/c(=[N+](/CC)CC(F)(F)F)cc-2oc2cc(N(CC)CC(F)(F)F)ccc12 ; [Cl-]] |
| 24 | 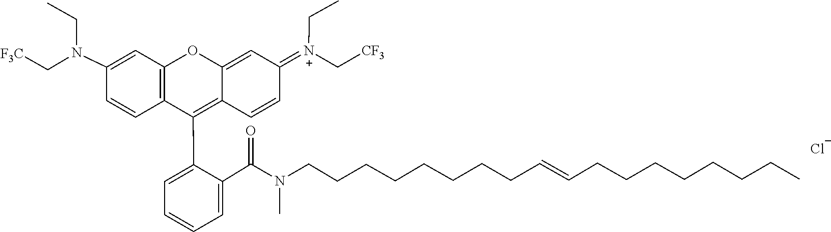 [SMILES: CCCCCCCC/C=C/CCCCCCCCN(C)C(=O)c1ccccc1-c1c2cc/c(=[N+](/CC)CC(F)(F)F)cc-2oc2cc(N(CC)CC(F)(F)F)ccc12 ; [Cl-]] |
| 25 | 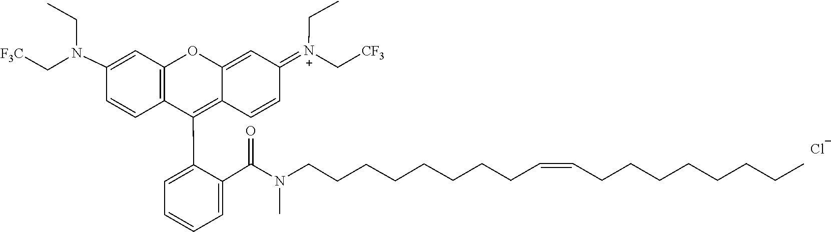 [SMILES: CCCCCCCC/C=C\CCCCCCCCN(C)C(=O)c1ccccc1-c1c2cc/c(=[N+](/CC)CC(F)(F)F)cc-2oc2cc(N(CC)CC(F)(F)F)ccc12 ; [Cl-]] |

In a preferred embodiments, the salt of formula (II) is selected from any one of the compounds 2, 3, 4, 6, 13, 15, 17 and 18, preferably any one of compounds 2, 15, 17 and 18.

In a preferred embodiment, the salt of formula (II) is compound 2.

The dyes of formula (I) and salts thereof (e.g. the compounds of formula (II)) of the invention may be prepared by methods known in the art, including chemical synthesis. For example, compounds of the invention may be prepared by reaction of a compound of formula (A)

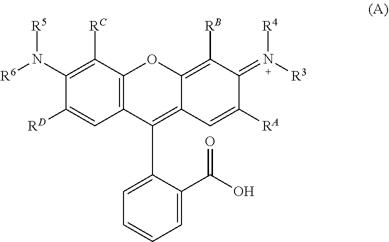

wherein p1 R^{A}, R^{B}, R^{C}, R^{D}, R³, R⁴, R⁵ and R⁶ are as defined for any compound of formula (I) described herein;
with a compound or formula (B),

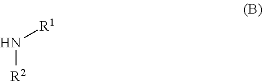

wherein R¹ and R² are as herein described. The reaction of the compound of formula (A) with the compound of formula (B) may occur in the presence of a suitable coupling agent, such as diisopropyl carbodiimide (DIC), dicyclohexyl carbodiimide (DCC), hydroxybenzotriazole (HOBt) and the like, optionally in the presence of a catalyst such as 4-dimethylaminopyridine (DMAP). Alternatively or additionally, the carboxylic acid moiety of the compound of formula (A) may be converted into an activated carboxylic acid, such as an acid chloride or a mixed anhydride. Acid chlorides may be prepared by reaction with a chlorinating reagent, such as posphoryl chloride (POCl₃) or thionyl chloride (SOCl₂). Mixed anhydrides may be prepared by reaction with dimethylanhydride.

In some embodiments, the dyes may be prepared using commercially available Rhodamine B as a starting material. The benzoic acid moiety of Rhodamine B may be converted to an acid halide, for example to an acid chloride by reacting with phosphoryl chloride (POCl₃), and the acid halide subsequently reacted with a suitable amine to provide the target dye. The dyes and salts of the invention may also be synthesised using methods analogous to those described in Example 1.

As shown in the Examples, the dyes of the invention fluoresce and are capable of acting as fluorescent markers that can be detected and tracked with a fluorimeter using methods known in the art. The dyes of the invention exhibit fluorescence in the visible range between 450-700 nm: as shown in the Examples, dyes 1-10 have a maxima of absorption of about 564 nm and a maxima of emission of about 588 nm. Accordingly, in some embodiments, the dye of the invention absorbs light at wavelength of about 544 nm to about 584 nm, or about 554 nm to about 574 nm, and fluoresces at a wavelength of about 568 nm to about 608 nm, or about 578 nm to about 598 nm. In preferred embodiments, the dye of the invention absorbs light at a wavelength of about 562 nm and emits light at a wavelength of about 583 nm.

### Labelled Extracellular Vesicles and Cells

The dyes of formula (I) and salts thereof of the invention are useful for labelling EVs and human cells.

EVs and cells comprise a lipid bilayer. The term “lipid bilayer” will be understood to mean a biological membrane consisting of two layers of phospholipid molecules, each phospholipid molecule containing a hydrophilic phosphate head and a hydrophobic lipid tail, where tail regions, being repelled by water and slightly attracted to each other, congregate together. Without wishing to be bound by theory, the present inventors hypothesise that the relatively hydrophobic moieties of R¹ and/or R² of the dyes of formula (I) associate with the lipid bilayer of EVs and cells, which thereby allows labelling of the EV/cell with the dye.

Accordingly, the present invention provides an extracellular vesicle (EV) labelled with a fluorescent dye of formula (I) as described herein.

The term “extracellular vesicle” (EV) as used herein is intended to encompass a vesicle within or outside a cell and which comprises a liquid or cytoplasm enclosed by a lipid bilayer. EVs typically contain cargo, which may be a therapeutic or drug cargo, for example one or more membrane proteins, cytosolic and nuclear proteins, extracellular matrix proteins, metabolites, and nucleic acids including DNA and RNA such as mRNA and non-coding RNA species. Examples of suitable types of cargo are described in Kalluri R and LeBleu V S (Science. 2020 Feb. 7; 367(6478)) The EV may be a naïve EV or an engineered EV. The term “naïve EV” as used herein will be understood to mean an unmodified EV that is naturally produced by cells. Examples of suitable cells from which naïve EVs can be derived include stem cells such as mesenchymal cells (MSCs), platelets and human induced pluripotent stem cells (hiPSCs), e.g., hiPSC derived neural stem cells. A naïve EV may have a lipid bilayer comprising one or more phospholipids which are substantially similar to the lipid bilayer of the cell from which the EV is derived. The term “engineered EV” as used herein will be understood to mean vesicles that have been modified to express a targeting molecule on their surface and/or to carry a specific drug cargo. EVs may be obtained by methods known in the art, for example methods by described in WO2018/112557 and WO2019/241836.

In some embodiments, the EV is selected from an exosome, an exomere, a microvesicle, an oncosome and an apoptotic body. In preferred embodiments, the EV is an exosome. The term “exosome” as used herein is intended to encompass EVs produced in the endosomal compartment of a eukaryotic cell. Exosomes typically have a diameter size of from about 40 nm to about 120 nm and contain protein and nucleic acid cargo. The term “exomere” as used herein is intended to encompass non-membranous EVs having a diameter size of less than about 50 nm. Exomeres typically have a diameter size of about 35 nm and contain protein and nucleic acid cargo. The term “microvesicle” as used herein is intended to encompass EVs released (shedded) from a cell plasma membrane. Microvesicles typically have a diameter size of from about 150 nm to about 1000 nm and contain protein and nucleic acid cargo. The term “oncosome” as used herein is intended to encompass EVs with pro-tumourigenic properties that are typically generated from the shedding of membrane blebs from cancer cells in more advanced stages of disease. Oncosomes (also called “large oncosomes”, LOs) typically have a diameter size of from about 1 μm to about 10 μm and contain oncogenic cargo including proteins. The term “apoptotic body” as used herein is intended to encompass EVs released from a cell plasma membrane during apoptosis. Apoptotic bodies typically have a diameter size of from about 500 nm to about 2000 nm and contain cargo including nuclear fractions and cell organelles.

The present invention also provides a cell labelled with a fluorescent dye of formula (I) as described herein. In some embodiments, the cell is a human cell. Examples of suitable human cells include normal human dermal fibroblasts (NHDF), human umbilical cord-derived mesenchymal stem cells (UC-MSCs) and bone marrow-derived MSCs (BM-MSCs).

As shown in the Examples, EVs and various human cells labelled with the fluorescent dye of the invention exhibit higher fluorescence compared to their unlabelled counterparts. This may advantageously allow EVs and cells labelled with the dyes of the invention to be detected and tracked using suitable instrumentation such as fluorimeters.

### Applications

The dyes of the invention may be useful as fluorescent markers for detecting and/or tracking labelled EVs and cells.

Accordingly, the present invention provides a method of labelling (or tagging) an extracellular vesicle (EV) or a cell, the method comprising combining the salt of formula (II) (e.g. a salt of a compound of formula (I)) described herein with the EV or the cell.

The present invention also provides a method of preparing a labelled extracellular vesicle (EV), the method comprising combining the salt of formula (II) (e.g. a salt of a compound of formula (I)) described herein with an EV to thereby label the EV with the compound of formula (I). The EV may be any suitable EV as described herein.

In these embodiments, the method may further comprise a step of providing an EV. The EV may be prepared or obtained by methods known in the art as described herein. Suitable methods for isolating EVs include those described in, for example, Sidhom K et al (Int. J. Mol. Sci. 2020, 21(18), 6466) and Zhang Y et al (Int J Nanomedicine. 2020;15:6917-6934). Suitable methods for purifying EVs include those described in, for example, WO 2018/112557 A1. The step of providing an EV may comprise, for example, obtaining an EV from a cell line and separating the EV from the cell. Put another way, the methods described herein may further comprise a step of isolating an EV from a cell to provide an aqueous solution comprising the EV.

The present invention also provides a method of preparing a labelled cell, the method comprising combining the salt of formula (II) (e.g. the salt of the compound of formula (I)) described herein with the cell to thereby label the cell with the compound of formula (I).

In these embodiments, the method may further comprise a step of providing a cell. The cell may be prepared or obtained by methods known in the art. The step of providing a cell may comprise, for example, culturing the cell. Suitable cell culture techniques for different cell types are available from the American Type Culture Collection (The Global Bioresource Centre) at https://www.atcc.org/.

The present invention also provides the use of the salt of the compound of formula (I) described herein for labelling an EV or a cell.

The present invention also provides a kit for fluorescently labelling an EV or a cell, or when used for fluorescently labelling an EV or a cell, the kit comprising:
a salt of the compound of formula (I) described herein; and optionally
instructions for its use to fluorescently label an extracellular vesicle or cell.

In any embodiment of the methods, uses and kits of the invention, the EV or the cell to be labelled may be any suitable EV or cell described herein.

The salt of the invention and the EV or cell are suitably combined such that the compound of formula (I) labels the EV or cell. For example, the EV or cell to be labelled may be incubated with the salt of the invention under suitable conditions and a suitable time period, for example at 37° C. for a period of about 1 hour. Typically the salt is added to an aqueous solution comprising the EVs and/or cells. However, in some embodiments, a solution of EVs and/or cells is added to an aqueous solution comprising the salt of the invention. The aqueous solution comprises water, and may comprise other water miscible solvents at levels capable of sustaining EV integrity and cell viability. Suitable water miscible solvents include dimethyl sulfoxide (DMSO), acetone, ethyl acetate (EtOAc), dimethylformamide (DMF), methanol (MeOH), ethanol (EtOH), and the like. The aqueous solution may comprise one or more additives selected from buffers, surfactants, culture medium, cryoprotectants dand combinations thereof.

Accordingly, in some embodiments, the step of combining the salt of formula (II) with an EV or with a cell comprises forming an aqueous solution of the salt of formula (II) and the EV or cell. The salt of formula (II) may be provided in any amount or at any concentration suitable for labelling the EV or cell. In some embodiments, the minimum concentration of the salt of formula (II) in the aqueous solution is at least about 0.02 μM, about 0.03 μM, about 0.05 μM, about 0.06 μM, about 0.07 μM, about 0.08 μM, about 0.09 μM, about 0.1 μM, about 0.11 μM, about 0.12 μM, about 0.13 μM, about 0.14 μM, about 0.15 μM, about 0.16 μM, about 0.17 μM, about 0.18 μM, about 0.19 μM, or about 0.2 μM. In some embodiments, the maximum concentration of the salt of formula (II) in the aqueous solution is not more than about 1 μM, about 2 μM, about 3 μM, about 4 μM, about 5 μM, about 6 μM, about 7 μM, about 8 μM, about 9 μM, about 10 μM, about 15 μM, or about 20 μM, based on the aqueous solution. In some embodiments, the concentration of the compound of formula (I) in the aqueous solution may be from any of these minimum concentration to any of these maximum concentrations, for example, from about 0.1 μM to about 5 μM or from about 0.2 μM to about 2 μM. In embodiments where the salt of formula (II) is for labelling an EV, the minimum amount of the salt of formula (II) may be at least about 60 molecules, about 90 molecules, about 150 molecules, about 180 molecules, about 210 molecules, about 240 molecules, about 270 molecules, about 300 molecules, about 330 molecules, about 360 molecules, about 390 molecules, about 420 molecules, about 450 molecules, about 480 molecules, about 510 molecules, about 540 molecules, about 570 molecules or about 600 molecules of salt per EV. In some embodiments, the maximum amount of the salt of formula (II) may be about 3000 molecules, about 6000 molecules, about 9000 molecules, about 12000 molecules, about 15000 molecules, about 18000 molecules, about 21000 molecules, about 24000 molecules, about 27000 molecules, about 30000 molecules, about 45000 molecules, or about 60000 molecules of salt per EV. In some embodiments, the salt of formula (II) may be in an amount ranging from any one of the minimum amounts to any one of the maximum amounts, for example, from about 300 molecules to about 15000 molecules or from about 600 molecules to about 6000 molecules of salt per EV. In embodiments where the salt of formula (II) is for labelling a cell, the amount of salt per cell may be orders of magnitude higher compared the amounts of salt per EV.

In some embodiments, the aqueous solution comprises an excess of the salt of formula (II) relative to the amount of salt labelling the EVs or cells. Accordingly, in some embodiments, the method further comprises purifying the solution to remove residual salt of formula (II). This purifying step may be achieved, for example by size-exclusion chromatography.

As shown in the Examples, the dye of the invention when labelled on EVs or cells is capable of fluorescence, which can be detected by suitable instrumentation. This advantageously allows the EVs and cells labelled with the dye to be detected and tracked using suitable instrumentation.

Accordingly, the present invention provides a method of detecting an extracellular vesicle (EV) or a cell, the method comprising:
exposing an EV or cell labelled with a fluorescent dye of formula (I) as described herein to a source of light selected to excite the fluorescent dye of formula (I); and
detecting fluorescence emission from the fluorescent dye.

The present invention also provides a method of tracking an extracellular vesicle (EV) or a cell, the method comprising:
exposing an EV or cell labelled with a fluorescent dye of formula (I) as described herein to a source of light selected to excite the fluorescent dye of formula (I); and
detecting fluorescence emission from the fluorescent dye, whereby detection of the fluorescence emission from the fluorescent dye allow for tracking of the labelled EV or cell.

The source of light may be any suitable light source capable of emitting light at a wavelength within the visible light range, that is, within the range of about 300 nm to about 1000 nm. In some embodiments, the light source emits light at a wavelength between about 450 to about 700 nm, preferably about 500 nm to about 650 nm, more preferably about 540 to about 590 nm.

The present invention also provides the use of the salt of the compound of formula (I) described herein for detecting and/or tracking an EV or a cell.

In any embodiment of the methods or uses of the invention, the labelled EV or labelled cell may be any suitable EV or cell described herein.

As shown in the Examples, EVs and various human cells labelled with the dyes of the invention are capable of being detected and imaged using conventional instrumentation. The detection and tracking of EVs and cells, especially EVs, may advantageously have applications in diagnostic and therapeutic research.

Accordingly, the present invention also provides a method of delivering a therapeutic cargo to a cell, the method comprising contacting a cell with an EV labelled with a fluorescent dye of formula (I) as described herein and comprising a therapeutic cargo as described herein. The cell and the labelled EV are suitably contacted such that the therapeutic cargo of the EV is delivered to the cell.

### EXAMPLES

The invention will be further described by way of non-limiting example(s). It will be understood to persons skilled in the art of the invention that many modifications may be made without departing from the spirit and scope of the invention.

### Example 1. Synthesis of Dyes

The dyes of the invention can be prepared according to methods known from the literature or methods known to those skilled in the art. The reagents used to prepare the dyes are in most cases commercially available or otherwise accessible by methods known from the literature. The preferred process for preparing the dyes of the invention is described by way of example on the basis of dyes 1-10.

### Synthesis of Compound 1

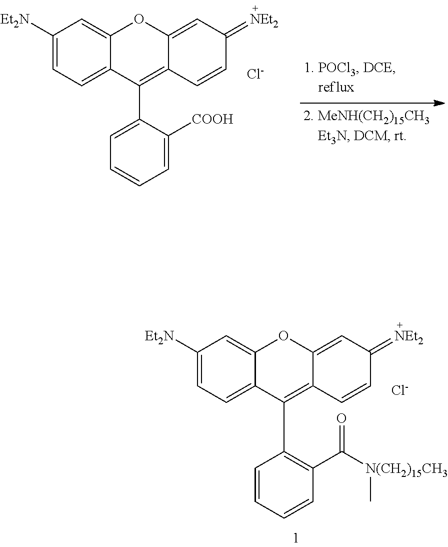

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and N-methylhexadecylamine (128.0 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with EtOAc (40 mL). The organic phase was washed was H₂O (20 mL), then brine (15 mL), before being dried (Na₂SO₄), passed through a 2 cm plug of silica and washed through with 10% MeOH/DCM (80 mL). Rotary evaporation gave 0.32 g of the crude amide as a tacky purple solid. The crude amide was purified by Biotage autocolumn chromatography to give Rhodamine B N-methylhexadecylamide (1) as a tacky purple solid (226.8 mg, 76%). ¹H NMR (400 MHz, CDCl₃) δ 7.68-7.60 (m, 2H_{R1}, 2H_{R2}), 7.55-7.49 (m, 1H_{R1}, 1H_{R2}), 7.33 (m, 1 H_{R1}, 1H_{R2}), 7.27 (d, J=9.4 Hz, 2H_{R1}), 7.22 (d, J=9.4 Hz, 2H_{R2}), 7.02 (dd, J=9.6, 2.5 Hz, 2H_{R1}), 6.89 (dd, J=9.5, 2.5 Hz, 2H_{R2}), 6.82 (d, J=2.4 Hz, 2H_{R2}), 6.74 (d, J=2.4 Hz, 2H_{R1}), 3.70-3.58 (m, 8H_{R1}, 8H_{R2}), 3.16 (t, J=7.4 Hz, 2H_{R1}), 3.02 (t, J=7.6 Hz, 2H_{R2}), 2.89 (s, 3H_{R1}), 2.69 (s, 3H_{R2}), 1.31 (app. t, J=7.1 Hz, 12H_{R1}, 12H_{R2}), 1.28-1.15 (m, 28H_{R1}, 28H_{R2}), 1.13-1.02 (m, 6H), 0.99-0.90 (m, 1H), 0.86 (m, 3H_{R1}, 3H_{R2}). NB: Rotamers are denoted R₁/R₂.

### Synthesis of Compound 2

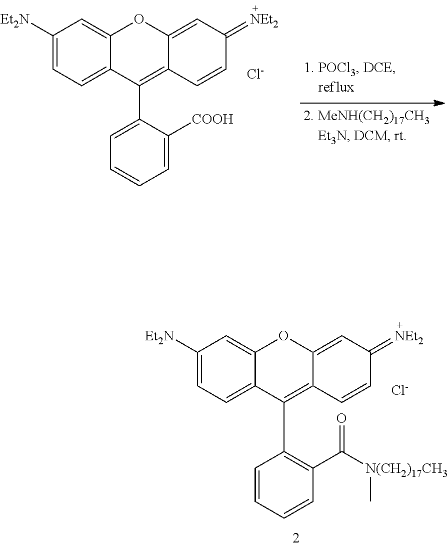

To a partial solution of Rhodamine B (0.500 g, 1.044 mmol, 1.0 eq.) in 1,2-dichloroethane (50 mL) was added POCl₃ (0.351 mL, 3.758 mmol, 3.6 eq.), and the bright purple mixture refluxed for four hours under N₂. Volatiles were removed by rotary evaporation to give the crude acyl halide as a bright purple film that was used without further purification. The residue was suspended in dry DCM (50 mL) and N-methyloctadecylamine (0.355 g, 1.253 mmol, 1.2 eq.) added in one portion. The resultant mixture was stirred for 15 minutes and then Et₃N (0.434 mL, 3.13 mmol, 3.0 eq.) added. The bright purple solution was stirred for 1 hour and monitored by TLC. The mixture was quenched with saturated aqueous NaHCO₃ (50 mL) and extracted with EtOAc (100 mL). The organic phase was washed was H₂O (50 mL), then brine (20 mL), before being dried (Na₂SO₄), passed through a 2 cm plug of silica and washed through with 10% MeOH/DCM (200 mL). Rotary evaporation gave 0.81 g of the crude amide as a tacky purple solid. The crude amide was purified by Biotage autocolumn chromatography to give Rhodamine B N-methyloctadecylamide (2) as a tacky purple solid (0.68 g, 87%). ¹H NMR (400 MHz, CDCl₃) δ 7.68-7.60 (m, 2H_{R1}, 2H_{R2}), 7.56-7.48 (m, 1H_{R1}, 1H_{R2}), 7.37-7.29 (m, 1H_{R1}, 1H_{R2}), 7.27 (d, J=9.6 Hz, 2H_{R1}), 7.22 (d, J=9.4 Hz, 2H_{R2}), 7.00 (dd, J=9.6, 2.4 Hz, 2H_{R1}), 6.88 (dd, J=9.4, 2.4 Hz, 2H_{R2}), 6.82 (d, J. 2.4 Hz, 2H_{R2}), 6.74 (d, J=2.4 Hz, 2H_{R1}), 3.72-3.54 (m, 8H_{R1}, 8H_{R2}), 3.16 (t, J=7.4 Hz, 2H_{R1}), 3.02 (t, J=7.9 Hz, 2H_{R2}), 2.89 (s, 3H_{R1}), 2.69 (s, 3H_{R2}), 1.31 (app. t, J=7.1Hz, 12H_{R1}, 12H_{R2}), 1.27-0.90 (m, 32H_{R1}, 32 H_{R2}), 0.86 (app. t, J=7.0 Hz, 3H_{R1}, 3H_{R2}). NB: Rotamers are denoted R₁/R₂.

### Synthesis of Compound 3

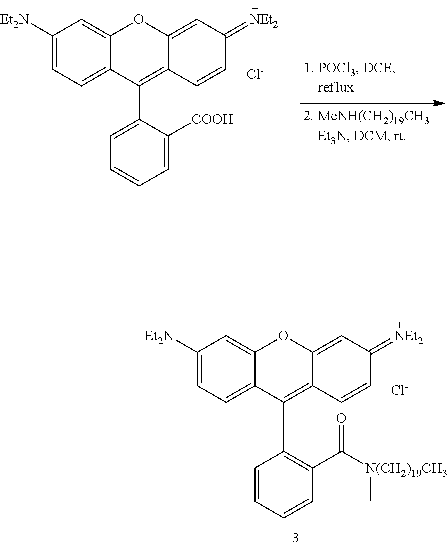

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and N-methylicosan-1-amine (156.11 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with EtOAc (40 mL). The organic phase was washed was H₂O (20 mL), then brine (15 mL), before being dried (Na₂SO₄), passed through a 2 cm plug of silica and washed through with 10% MeOH/DCM (80 mL) and 10% MeOH/Et₂O(80 mL). Rotary evaporation gave 0.64 g of the crude amide as a tacky purple solid. The crude amide was purified by Biotage autocolumn chromatography to give Rhodamine B N-methylicosaneamide (3) as a tacky purple solid (188.0 mg, 58%). ¹H NMR (400 MHz, CDCl₃) δ 7.67-7.58 (m, 2H_{R1}, 2H_{R2}), 7.54-7.47 (m, 1H_{R1}, 1H_{R2}), 7.35-7.28 (m, 1H_{R1}, 1H_{R2}), 7.25 (d, J=9.6 Hz, 2H_{R1}), 7.20 (d, J=9.6 Hz, 2H_{R2}), 7.00 (dd, J=9.6, 2.5 Hz, 2H_{R1}), 6.87 (dd, J=9.5, 2.5 Hz, 2H_{R2}), 6.78 (d, J=2.4 Hz, 2H_{R2}), 6.71 (d, J=2.4 Hz, 2H_{R1}), 3.71-3.55 (m, 8H_{R1}, 8H_{R2}), 3.14 (t, J=7.3 Hz, 2H_{R1}), 3.01 (m, 2H_{R2}), 2.87 (s, 3H_{R1}), 2.67 (s, 3H_{R2}), 1.30 (app. t, J=7.1Hz, 12H_{R1}, 12H_{R2}), 1.26-1.11 (m, 36H_{R1}, 36H_{R2}), 1.11-1.00 (m, 6H), 0.96-0.88 (m, 1H), 0.87-0.81 (m, 3H_{R1}, 3H_{R2}). NB: Rotamers are denoted R₁/R₂.

### Synthesis of Compound 4

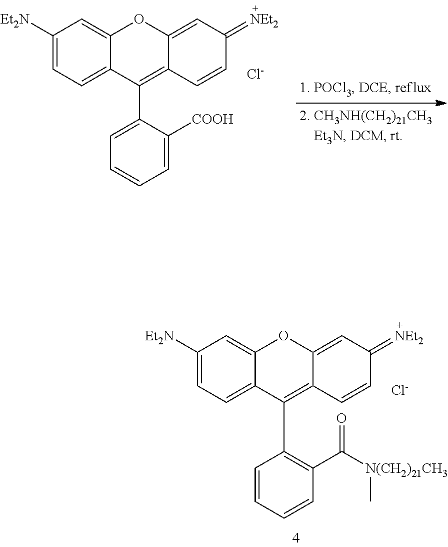

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and N-methyldocosylamine (170.16 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (80 mL) and 10% MeOH/Et₂O (80 mL). Rotary evaporation gave 0.27 g of the crude amide as a tacky purple solid. The crude amide was purified by Biotage autocolumn chromatography to give Rhodamine B N-pentyltetradecanamide (4) as a tacky purple solid (291.1 mg, 73%). ¹H NMR (400 MHz, CDCl₃) δ 7.68-7.59 (m, 2H_{R1}, 2H_{R2}), 7.54-7.49 (m, 1H_{R1}, 1H_{R2}), 7.36-7.31 (m, 1H_{R1}, 1H_{R2}), 7.27 (d, J=9.5 Hz, 2H_{R1}), 7.22 (d, J=9.5 Hz, 2H_{R2}), 7.03 (dd, J=9.6, 2.5 Hz, 2H_{R1}), 6.89 (dd, J=9.5, 2.5 Hz, 2H_{R2}), 6.81 (d, J=2.4 Hz, 2H_{R2}), 6.73 (d, J=2.5 Hz, 2H_{R1}), 3.69-3.57 (m, 8H_{R1}, 8H_{R2}), 3.16 (t, J=7.4 Hz, 2H_{R1}), 3.08-2.98 (m, 2H_{R2}), 2.89 (s, 3H_{R1}), 2.69 (s, 3H_{R2}), 1.31 (app. t, J=7.1Hz, 12H_{R1}, 12H_{R2}), 1.28-1.12 (m, 40H_{R1}, 40H_{R2}), 1.12-1.00 (m, 6H), 0.99-0.89 (m, 1H), 0.89-0.78 (m, 3H_{R1}, 3H_{R2}). NB: Rotamers are denoted R₁/R₂.

### Synthesis of Compound 5

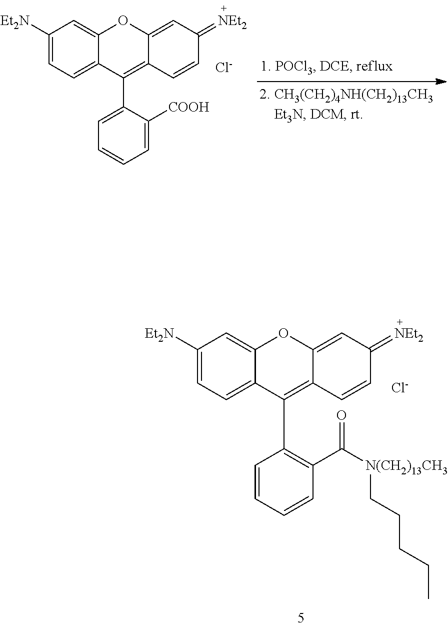

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and N-pentyltetradecan-1-amine (142.05 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (80 mL) and 10% MeOH/Et₂O (80 mL). Rotary evaporation gave 0.35 g of the crude amide as a tacky purple solid. The crude amide was purified by Biotage autocolumn chromatography to give Rhodamine B N-pentyltetradecanamide (5) as a tacky purple solid (211.5 mg, 68%). ¹H NMR (400 MHz, Chloroform-d) δ 7.68-7.61 (m, 2H), 7.54-7.47 (m, 1H), 7.38-7.32 (m, 1H), 7.24 (d, J=9.7 Hz, 2H), 6.91 (ddd, J=9.7, 5.3, 2.3 Hz, 2H), 6.75 (dd, J=5.5, 2.3 Hz, 2H), 3.62 (dq, J=11.3, 7.3 Hz, 8H), 3.20-3.02 (m, 2H), 2.90 (q, J=7.0 Hz, 2H), 1.49-1.36 (m, 3H), 1.31 (t, J=7.0 Hz, 12H), 1.27-0.95 (m, 20H), 0.93-0.77 (m, 9H), 0.71 (t, J=7.3 Hz, 3H).

### Synthesis of Compound 6

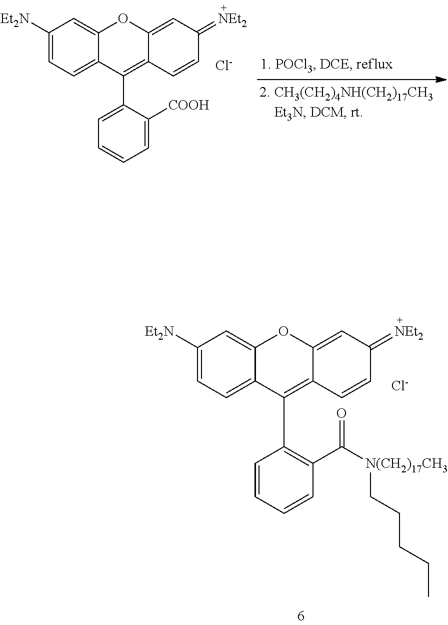

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and N-pentyloctadecan-1-amine (170.16 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (80 mL) and 10% MeOH/Et₂O (80 mL). Rotary evaporation gave 0.41 g of the crude amide as a tacky purple solid. The crude amide was purified by Biotage autocolumn chromatography to give Rhodamine B N-pentyloctadecanamide (6) as a tacky purple solid (281.4 mg, 84%). ¹H NMR (400 MHz, Chloroform-d) δ 7.70-7.61 (m, 2H), 7.55-7.48 (m, 1H), 7.39-7.33 (m, 1H), 7.27-7.23 (m, 2H), 6.91 (ddd, J=9.6, 5.8, 2.5 Hz, 2H), 6.77 (dd, J=6.1, 2.5 Hz, 2H), 3.65 (dq, J=11.9, 7.4 Hz, 8H), 3.10 (s, 2H), 2.91 (q, J=7.2 Hz, 2H), 1.42 (t, J=7.5 Hz, 2H), 1.31 (t, J=7.1Hz, 12H), 1.28-0.95 (m, 28H), 0.91-0.80 (m, 9H), 0.72 (t, J=7.3 Hz, 2H).

### Synthesis of Compound 7

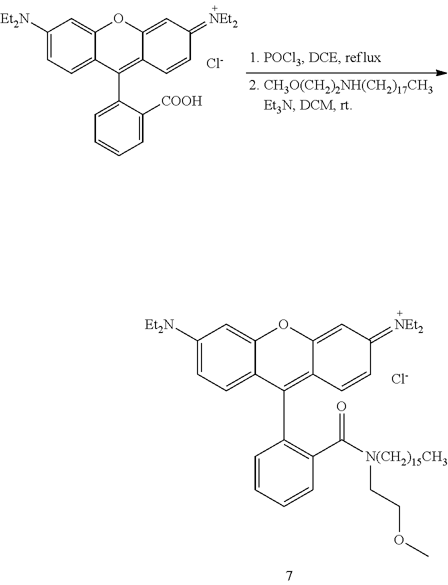

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and N-(2-methoxyethyl)hexadecan-1-amine (150.07 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (80 mL). Rotary evaporation gave 0.35 g of the crude amide as a tacky purple solid. The crude amide was purified by Biotage autocolumn chromatography to give 162.0 mg of Rhodamine B N-ethyl methyl ether hexadecanamide (7) as a tacky purple solid. 1H NMR (400 MHz, CDCl₃) δ 7.73-7.57 (m, 2H_{R1}, 2H_{R2}), 7.55-7.50 (m, 1H_{R1}, 1H_{R2}), 7.39-7.29 (m, 1H_{R1}, 1H_{R2}), 7.29-7.22 (m, 2H_{R1}, 2H_{R2}), 7.03 (d, J=9.5 Hz, 2H_{R1}), 6.88 (dd, J=9.5, 2.4 Hz, 2H_{R2}), 6.82 (d, J=2.4 Hz, 2H_{R2}), 6.72 (d, J=2.4 Hz, 2H_{R1}), 3.72-3.55 (m, 8H_{R1}, 8H_{R2}), 3.42 (t, J=5.1Hz, 1H), 3.37-3.23 (m, 3H); 3.19-3.09 (m, 2H_{R1}), 3.00-2.88 (m, 2HR₂, 3HR₁, 3H_{R2}), 1.36-1.28 (m, 12H_{R1}, 12H_{R2}), 1.27-1.12 (m, 28H_{R1}, 28H_{R2}), 0.93-0.82 (m, 6H). NB: Rotamers are denoted R₁/R₂.

### Synthesis of Compound 8

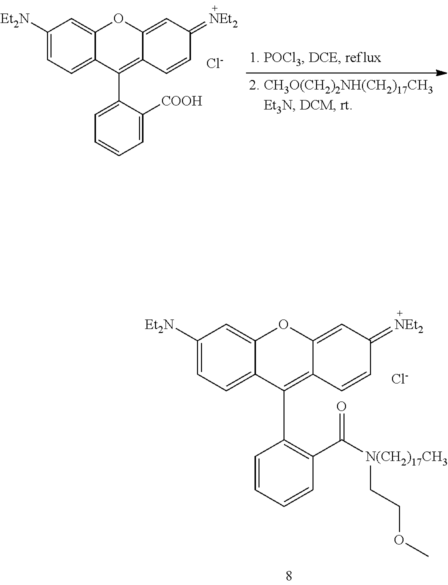

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and N-(2-methoxyethyl)octadecan-1-amine (164.12 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with EtOAc (40 mL). The organic phase was washed was H₂O (20 mL), then brine (15 mL), before being dried (Na₂SO₄), passed through a 2 cm plug of silica and washed through with 10% MeOH/DCM (80 mL) and 10% MeOH/Et₂O(80 mL). Rotary evaporation gave 0.53 g of the crude amide as a tacky purple solid. The crude amide was purified by Biotage autocolumn chromatography to give Rhodamine B N-ethyl methyl ether octadecanamide (8) as a tacky purple solid (175.9 mg, 53%). ¹H NMR (400 MHz, CDCl₃) δ 7.75-7.58 (m, 2H_{R1}, 2H_{R2}), 7.57-7.50 (m, 1H_{R1}, 1H_{R2}), 7.40-7.30 (m, 1H_{R1}, 1H_{R2}), 7.30-7.23 (m, 2H_{R1}, 2H_{R2}), 7.00 (d, J=9.6 Hz, 2H_{R1}), 6.90 (dd, J=9.5, 2.4 Hz, 2H_{R2}), 6.83 (d, J=2.4 Hz, 2H_{R2}), 6.73 (d, J=2.4 Hz, 2H_{R1}), 3.77-3.55 (m, 8H_{R1}, 8H_{R2}), 3.44 (t, J=5.1Hz, 1H), 3.30 (s, 3H); 3.14 (m, 2H_{R1}), 2.96 (m, 2HR₂, 3H_{R1}, 3H_{R2}), 1.36-1.29 (m, 12H_{R1}, 12H_{R2}), 1.29-1.13 (m, 32H_{R1}, 32H_{R2}), 0.93-0.82 (m, 6H). NB: Rotamers are denoted R₁/R₂.

### Synthesis of Compound 9

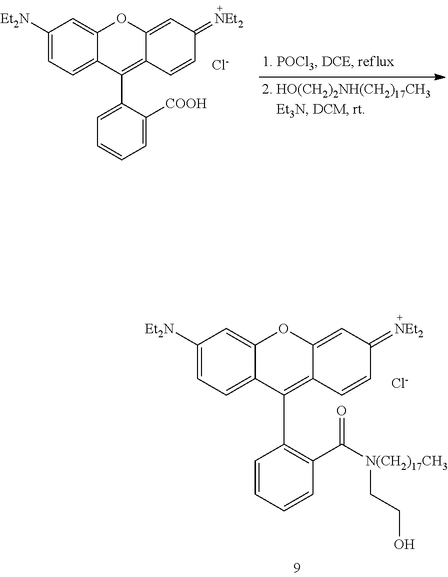

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and 2-(octadecylamino)ethan-1-ol (157.10 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (80 mL). Rotary evaporation gave 0.33 g of the crude amide as a tacky purple solid. The crude amide was purified by Biotage autocolumn chromatography to give Rhodamine B N-(2-hydroxyethyl)octadecanamide (9) as a tacky purple solid (144.2 mg, 45%). ¹H NMR (400 MHz, Chloroform-d) δ 7.72-7.54 (m, 3H), 7.42 (d, J=9.5 Hz, 1H), 7.26 (s, 2H), 7.20-7.10 (m, 1H), 6.90-6.83 (m, 1H), 6.66 (d, J=2.4 Hz, 2H), 3.74-3.61 (m, 8H), 3.61-3.50 (m, 2H), 3.47 (t, J=6.6 Hz, 1H), 3.33 (s, 1H), 3.21 (t, J=7.4 Hz, 1H), 3.11 (qd, J=7.3, 4.8 Hz, 1H), 2.96 (t, J=7.5 Hz, 1H), 1.41 (t, J=7.3 Hz, 2H), 1.36-1.28 (m, 12H), 1.28-1.02 (m, 32H), 1.02-0.92 (m, 1H), 0.91-0.82 (m, 3H).

### Synthesis of Compound 10

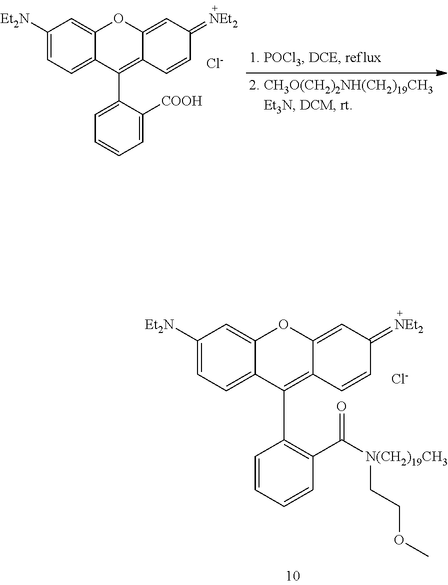

To a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (20 mL) and N-(2-methoxyethyl)icosan-1-amine (178.18 mg, 0.501 mmol, 1.2 eq.) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (80 mL) and 10% MeOH/Et₂O (80 mL). Rotary evaporation gave 0.36 g of the crude amide as a tacky purple solid. The crude amide was purified by Biotage autocolumn chromatography to give 154.1 mg of Rhodamine B N-ethyl methyl ether icosaneamide (10) as a tacky purple solid (154.1 mg, 45%). ¹H NMR (400 MHz, CDCl₃) δ 7.75-7.58 (m, 2H_{R1}, 2H_{R2}), 7.57-7.50 (m, 1H_{R1}, 1H_{R2}), 7.41-7.34 (m, 1H_{R1}, 1H_{R2}), 7.34-7.23 (m, 2H_{R1}, 2H_{R2}), 7.08 (d, J=9.4 Hz, 2H_{R1}), 6.90 (dd, J=9.5, 2.4 Hz, 2H_{R2}), 6.85 (d, J=2.4 Hz, 2H_{R2}), 6.74 (d, J=2.4 Hz, 2H_{R1}), 3.74-3.57 (m, 8H_{R1}, 8H_{R2}), 3.45 (t, J=4.9 Hz, 1H), 3.37-3.28 (s, 3H); 3.20-3.09 (m, 2H_{R1}), 3.01-2.90 (m, 2HR₂, 3H_{R1}, 3H_{R2}), 1.37-1.29 (m, 12H_{R1}, 12H_{R2}), 1.29-1.05 (m, 36H_{R1}, 36H_{R2}), 0.94-0.80 (m, 6H). NB: Rotamers are denoted R₁/R₂.

### Synthesis of Compound 11

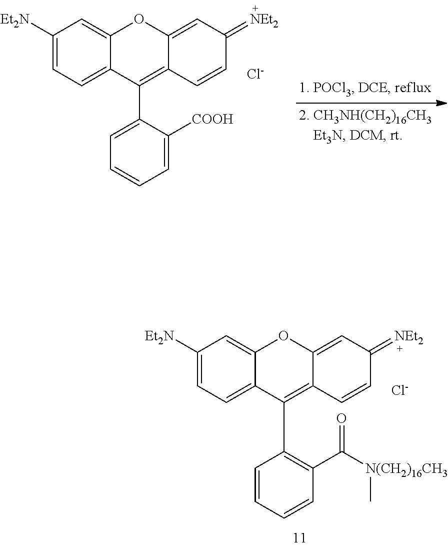

To a 250 mL 1N RBF containing a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (17 mL) and N-methylhenicosan-1-amine (163.14 mg, 0.501 mmol, 1.2 eq.) dissolved in DCM (3 mL) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by Biotage autocolumn chromatography, and following rotary evaporation, dried in the vacuum oven (40° C.) overnight to give 315.1 mg of Rhodamine B N-methylhenicosaneamide (11) as a tacky purple solid (315.1 mg, 96%). ¹H NMR (400 MHz, CDCl₃) δ 7.70-7.61 (m, 2H_{R1}, 2H_{R2}), 7.56-7.50 (m, 1H_{R1}, 1H_{R2}), 7.38-7.32 (m, 1H_{R1}, 1H_{R2}), 7.29 (d, J=9.6 Hz, 2H_{R1}), 7.23 (d, J=9.5 Hz, 2H_{R2}) 7.07 (dd, J=9.6, 2.4 Hz, 2H_{R1}), 6.92 (dd, J=9.5, 2.4 Hz, 2H_{R2}), 6.86 (d, J=2.4 Hz, 2H_{R1}) 6.76 (d, J=2.4 Hz, 2H_{R2}), 3.71-3.59 (m, 8H_{R1}, 8H_{R2}), 3.17 (t, J=7.4 Hz, 2H), 2.91 (s, 2H), 1.33 (t, J=7.1Hz, 12H), 1.30-1.13 (m, 38H), 1.13-1.02 (m, 4H), 0.99-0.91 (m, 1H), 0.86 (t, 4H).

### Synthesis of Compound 12

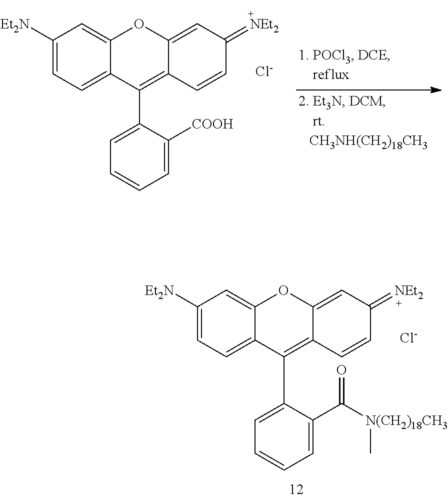

To a 250 mL 1N RBF containing a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (17 mL) and N-methylnonadecylamine (149.08 mg, 0.501 mmol, 1.2 eq.) dissolved in DCM (3 mL) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by Biotage autocolumn chromatography, and following rotary evaporation, dried in the vacuum oven (40° C.) overnight to give 229.6 mg of Rhodamine B N-methylnonadecylamide (12) as a tacky purple solid (229.6 mg, 72%). ¹H NMR (400 MHz, CDCl₃) δ 7.70-7.61 (m, 2H_{R1}, 2H_{R2}), 7.58-7.50 (m, 1H_{R1}, 1H_{R2}), 7.38-7.33 (m, 1H_{R1}, 1H_{R2}), 7.29 (d, J=9.6 Hz, 2H_{R1}), 7.23 (d, J=9.4 Hz, 2H_{R2}) 7.06 (dd, J=9.5, 2.4 Hz, 2H_{R1}), 6.91 (dd, J=9.6, 2.3 Hz, 2H_{R2}), 6.85 (d, J=2.4 Hz, 2H_{R1}) 6.75 (d, J=2.3 Hz, 2H_{R2}), 3.72-3.58 (m, 8H_{R1}, 8H_{R2}), 3.17 (t, J=7.4 Hz, 2H), 2.91 (s, 2H), 2.19 (s, 4H), 1.33 (t, J=7.1Hz, 12H), 1.29-1.14 (m, 34H), 1.14-1.02 (m, 4H), 1.01-0.91 (m, 1H), 0.87 (t, J=6.8 Hz, 4H).

### Synthesis of Compound 13

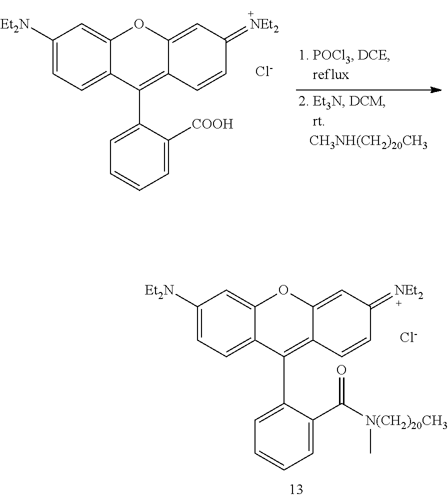

To a 250 mL 1N RBF containing a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (17 mL) and N-methylhenicosan-1-amine (163.14 mg, 0.501 mmol, 1.2 eq.) dissolved in DCM (3 mL) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by Biotage autocolumn chromatography, and following rotary evaporation, dried in the vacuum oven (40° C.) overnight to give 315.1 mg of Rhodamine B N-methylhenicosaneamide (13) as a tacky purple solid (315.1 mg, 96%) ¹H NMR (400 MHz, CDCl₃) δ 7.70-7.61 (m, 2H_{R1}, 2H_{R2}), 7.56-7.50 (m, 1H_{R1}, 1H_{R2}), 7.38-7.32 (m, 1H_{R1}, 1H_{R2}), 7.29 (d, J=9.6 Hz, 2H_{R1}), 7.23 (d, J=9.5 Hz, 2H_{R2}) 7.07 (dd, J=9.6, 2.4 Hz, 2H_{R1}), 6.92 (dd, J=9.5, 2.4 Hz, 2H_{R2}), 6.86 (d, J=2.4 Hz, 2H_{R1}) 6.76 (d, J=2.4 Hz, 2H_{R2}), 3.71-3.59 (m, 8H_{R1}, 8H_{R2}), 3.17 (t, J=7.4 Hz, 2H), 2.91 (s, 2H), 1.33 (t, J=7.1Hz, 12H), 1.30-1.13 (m, 38H), 1.13-1.02 (m, 4H), 0.99-0.91 (m, 1H), 0.86 (t, 4H).NB: Rotamers are denoted R₁/R₂.

### Synthesis of Compound 14

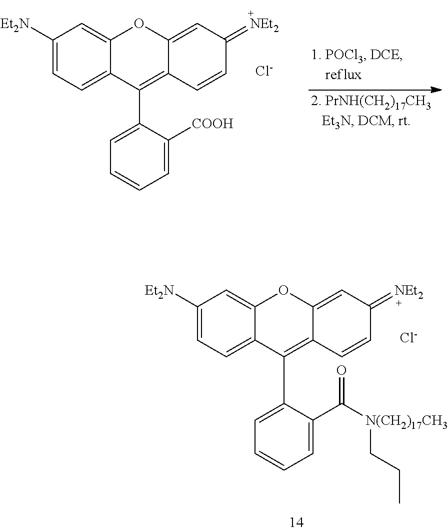

To a 250 mL 1N RBF containing a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (17 mL) and N-propyloctadecan-1-amine (156.11 mg, 0.501 mmol, 1.2 eq.) dissolved in DCM (3 mL) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by Biotage autocolumn chromatography, and following rotary evaporation, dried in the vacuum oven (40° C.) overnight to give 235.3 mg of Rhodamine B N-propyloctadecylamide (14) as a tacky purple solid (235.3 mg, 73%) ¹H NMR (400 MHz, CDCl₃) δ 7.69-7.61 (m, 2H_{R1}, 2H_{R2}), 7.55-7.48 (m, 1H_{R1}, 1H_{R2}), 7.39-7.32 (m, 1H_{R1}, 1H_{R2}), 7.28-7.22 (m, 2H_{R1}, H_{R2}) 6.94 (dd, J=9.5, 2.4 Hz, 2H_{R1}), 6.89 (dd, J=9.6, 2.4 Hz, 2H_{R2}), 6.79 (d, J=2.4 Hz, 2H_{R1}) 6.75 (d, J=2.4 Hz, 2H_{R2}), 3.71-3.54 (m, 8H_{R1}, 8H_{R2}), 3.14-3.03 (m, 2H), 2.91 (t, J=7.2 Hz, 2H), 2.59 (s, 1H); 2.05-1.84 (m, 1H), 1.55-1.36 (m, 2H), 1.31 (t, J=7.1Hz, 11H), 1.28-1.04 (m, 35H), 1.05-0.93 (m, 1H), 0.92-0.82 (m, 6H), 0.79 (t, J=7.3 Hz, 1H). NB: Rotamers are denoted R₁/R₂.

### Synthesis of Compound 15

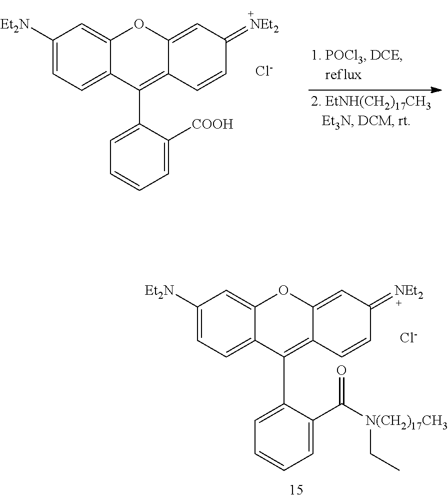

To a 250 mL 1N RBF containing a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (17 mL) and N-ethyloctadecan-1-amine (149.09 mg, 0.501 mmol, 1.2 eq.) dissolved in DCM (3 mL) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed with H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by Biotage autocolumn chromatography and following rotary evaporation, dried in the vacuum oven (40° C.) overnight to give 123.0 mg of Rhodamine B N-ethyloctadecylamide (15) as a tacky purple solid (123.0 mg, 39%). ¹H NMR (400 MHz, CDCl₃) δ 7.68-7.60 (m, 2H_{R1}, 2H_{R2}), 7.55-7.46 (m, 1H_{R1}, 1H_{R2}), 7.39-7.30 (m, 1H_{R1}, 1H_{R2}), 7.27-7.20 (m [2×d] 2H_{R1}, 2H_{R2}), 6.96 (dd, J=9.5, 2.4 Hz, 2H_{R1}), 6.88 (d, J=2.4 Hz, 2H_{R2}), 6.78 (d, J=2.4 Hz, 2H_{R1}) 6.72 (d, J=2.3 Hz, 2H_{R2}), 3.68-3.53 (m, 8H_{R1}, 8H_{R2}), 3.23-3.03 (m, 3H), 3.03-2.78 (m, 1H), 1.48 -1.37 (m, 1H), 1.30 (t, J=7.1Hz, 12H), 1.27-1.10 (m, 34H), 1.05 (h, J=6.6 Hz, 2H), 0.96-0.87 (m, 4H), 0.84 (t, J=6.7 Hz, 4H), 0.55 (t, J=7.0 Hz, 1H). NB: Rotamers are denoted R₁/R₂.

### Synthesis of Compound 16

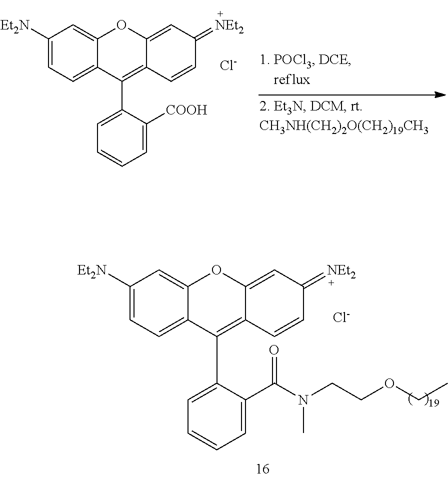

To a 250 mL 1N RBF containing a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (17 mL) and 2-(icosyloxy)-N-methylethanamine (178.18 mg, 0.501 mmol, 1.2 eq.) dissolved in DCM (3 mL) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by Biotage autocolumn chromatography, and following rotary evaporation, dried in the vacuum oven (40° C.) overnight to give 336.8 mg of rhodamine B N-(2-(icosyloxy)ethyl)-N-methylamide (16) as a tacky purple solid (336.8 mg, 99%). ¹H NMR (400 MHz, Chloroform-d) δ 7.81 -7.74 (m, 1H), 7.69-7.63 (m, 2H), 7.63-7.57 (m, 1H), 7.57-7.50 (m, 1H), 7.38-7.31 (m, 1H), 7.30-7.21 (m, 5H), 7.02-6.93 (m, 3H), 6.80-6.75 (m, 3H), 3.71-3.55 (m, 11H), 3.40-3.31 (m, 4H), 3.22-3.11 (m, 4H), 2.93 (s, 3H), 2.72 (s, 2H), 1.42-1.35 (m, 2H), 1.31 (t, J=7.1Hz, 17H), 1.27-1.15 (m, 59H), 0.85 (t, J=6.7 Hz, 5H).

### Synthesis of Compound 17

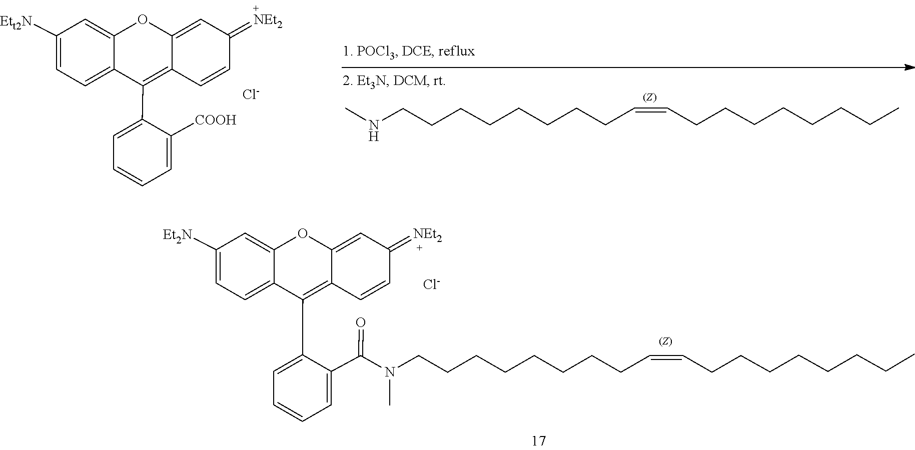

To a 250 mL 1N RBF containing a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (17 mL) and (Z)-N-methyloctadec-9-en-1-amine (141.05 mg, 0.501 mmol, 1.2 eq.) dissolved in DCM (3 mL) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave 0.33 g of the crude amide as a tacky purple solid. The crude amide was purified by Biotage autocolumn chromatography, and following rotary evaporation, dried in the vacuum oven (40° C.) overnight to give 251.2 mg of Rhodamine B N-methyloleylamide (17) as a tacky purple solid (251.2 mg, 81%). ¹H NMR (400 MHz, CDCl₃) δ 7.68-7.58 (m, 2H_{R1}, 2H_{R2}), 7.55-7.46 (m, 1H_{R1}, 1H_{R2}), 7.36-7.28 (m, 1H_{R1}, 1H_{R2}), 7.28-7.16 (m, 2H_{R1}, 2H_{R2}), 7.00 (dd, J=9.5, 2.4 Hz, 2H_{R1}), 6.87 (d, J=2.4 Hz, 2H_{R2}), 6.78 (d, J=2.4 Hz, 2H_{R2}) 6.72 (d, J=2.3 Hz, 2H_{R1}), 5.40-5.23 (m, 2H_{R1}, 2H_{R2}); 3.72-3.52 (m, 8H_{R1}, 8H_{R2}), 3.14 (t, J=7.4 Hz, 2H), 3.01 (t, J=7.7 Hz, 1H); 2.88 (s, 2H), 2.68 (s, 1H); 2.04-1.85 (m, 4H), 1.49-1.35 (m, 1H), 1.35-1.15 (m, 28H), 1.15-0.99 (m, 3H), 0.98-0.87 (m, 2H), 0.84 (t, J=6.7 Hz, 3H). NB: Rotamers are denoted R₁/R₂.

### Synthesis of Compound 18

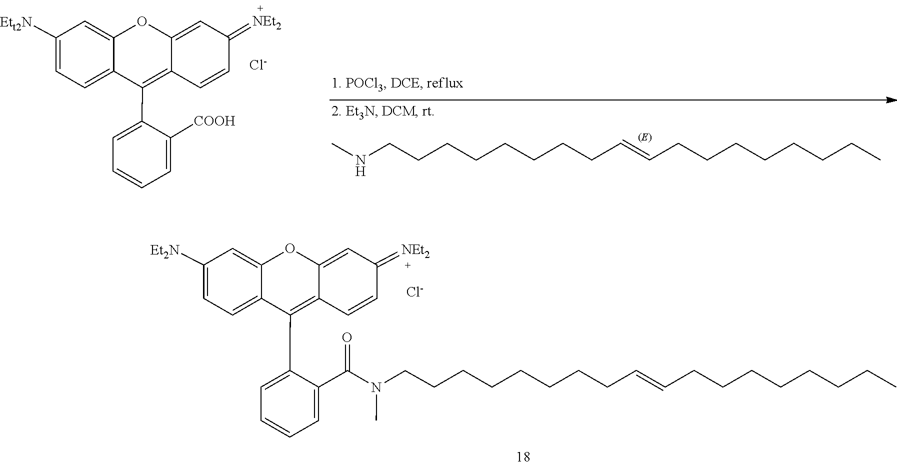

To a 250 mL 1N RBF containing a partial solution of Rhodamine B (0.600 g, 1.253 mmol, 3.0 eq.) in 1,2-dichloroethane (60 mL) was added POCl₃ (422 μL, 4.510 mmol, 10.8 eq.). The bright purple mixture was refluxed for four hours under N₂, then allowed to cool to ambient temperature. The mixture was divided into three separate 100 mL RBFs and the volatiles removed by rotary evaporation to give the crude acyl halide as a bright purple film that was used without further purification. The residue in one flask was suspended in dry DCM (17 mL) and (E)-N-methyloctadec-9-en-1-amine (141.05 mg, 0.501 mmol, 1.2 eq.) dissolved in DCM (3 mL) was added in one portion. The resultant mixture was stirred for 15 minutes and then Et₃N (173.5 μL, 1.252 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by Biotage autocolumn chromatography, and following rotary evaporation, dried in the vacuum oven (40° C.) overnight to give 245.6 mg of Rhodamine B N-methylelaidylamide (18) as a tacky purple solid (245.6 mg, 79%). ¹H NMR (400 MHz, CDCl₃) δ 7.67-7.58 (m, 2H_{R1}, 2H_{R2}), 7.53-7.47 (m, 1H_{R1}, 1H_{R2}), 7.36-7.28 (m, 1H_{R1}, 1H_{R2}), 7.26-7.17 (m, 2H_{R1}, 2H_{R2}), 6.98 (d, J=9.0, 2H_{R1}), 6.86 (s, 2H_{R2}), 6.78 (s, 2H_{R2}) 6.72 (d, J=2.3 Hz, 2H_{R1}), 5.40-5.26 (m, 2H_{R1}, 2H_{R2}); 3.75-3.42 (m, 8H_{R1}, 8H_{R2}), 3.13 (t, J=7.3 Hz, 2H), 3.00 (t, J=7.7 Hz, 1H); 2.86 (s, 2H), 2.67 (s, 1H); 2.02-1.83 (m, 4H), 1.49-1.38 (m, 1H), 1.32-1.26 (m, 12H), 1.26-1.12 (m, 26H), 1.12-0.98 (m, 3H), 0.96-0.86 (m, 1H), 0.83 (t, J=6.7 Hz, 3H). NB: Rotamers are denoted R₁/R₂.

### Synthesis of Compound 19

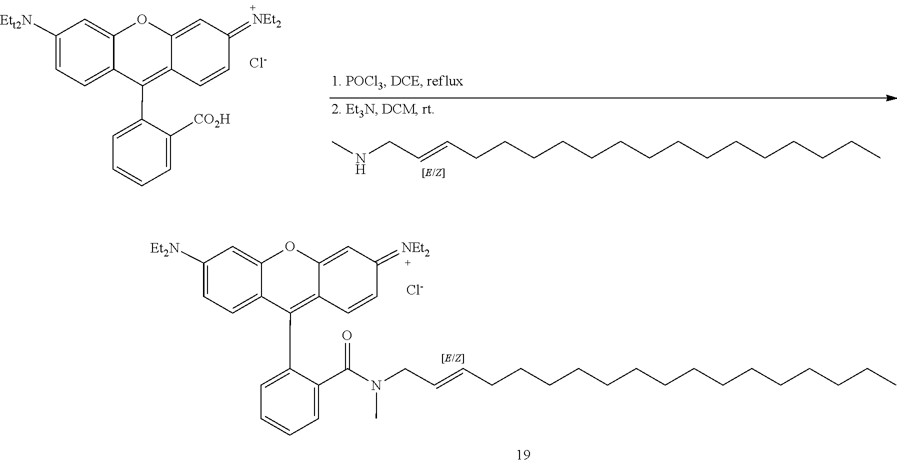

To a 250 mL 1N RBF containing a solution of Rhodamine B (2.00 g, 4.18 mmol, 1.0 eq.) in 1,2-dichloroethane (170 mL) was added POCl₃ (1.40 mL, 15.0 mmol, 3.6 eq.). The bright purple mixture was refluxed for four hours under N₂, then allowed to cool to ambient temperature. The volatiles were removed by rotary evaporation to give the crude acyl halide as a bright purple film that was used without further purification. The residue was dissolved in 100 mL of DCM and an aliquot of 26.3 ml added to a stirred solution of (E/Z)-N-methyloctadec-2-en-1-amine (0.37 g, 1.3 mmol, 1.2 eq) in DCM (10 ml). The resultant mixture was stirred for 15 minutes and then Et₃N (455 μL, 3.3 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by column chromatography eluting with gradient solvent system of DCM/ MeOH (5% DCM/MeOH-10% MeOH/ DCM), and following rotary evaporation, dried in the vacuum oven (40° C.) overnight to give 630 mg of N-(6-(diethylamino)-9-(2-(methyl(octadec-2-en-1-yl)carbamoyl)phenyl)-3H-xanthen-3-ylidene)-N-ethylethanaminium chloride (19) as a tacky purple solid (630 mg, 81%). ¹H NMR (400 MHz, CDCl₃) δ 7.70-7.60 (m, 2H_{R1}, 2H_{R2}), 7.57-7.53 (m, 1H_{R1}, 1H_{R2}), 7.36-7.30 (m, 1H_{R1}, 1H_{R2}), 7.26-7.19 (m, 2H_{R1}, 2H_{R2})) , 7.02 (dd, J=9.6, 2.2 Hz, 2H_{R1}), 6.95 (d, J=2.5 Hz, 2H_{R2}), 6.82 (d, J=2.5 Hz, 2H_{R2}), 5.62-4.85 (m, 2H_{R1}, 2H_{R2}); 3.75-3.42 (m, 10H_{R1}, 10H_{R2}), 2.84 (s, 2H), 2.70 (s, 2H); 2.10-1.90 (m, 4H), 1.85-1.75 (m, 1H), 1.5-1.00 (m, 38 H), 0.87 (t, J=6.7 Hz, 3H). NB: Rotamers are denoted R₁/R₂.

### Synthesis of Compound 20

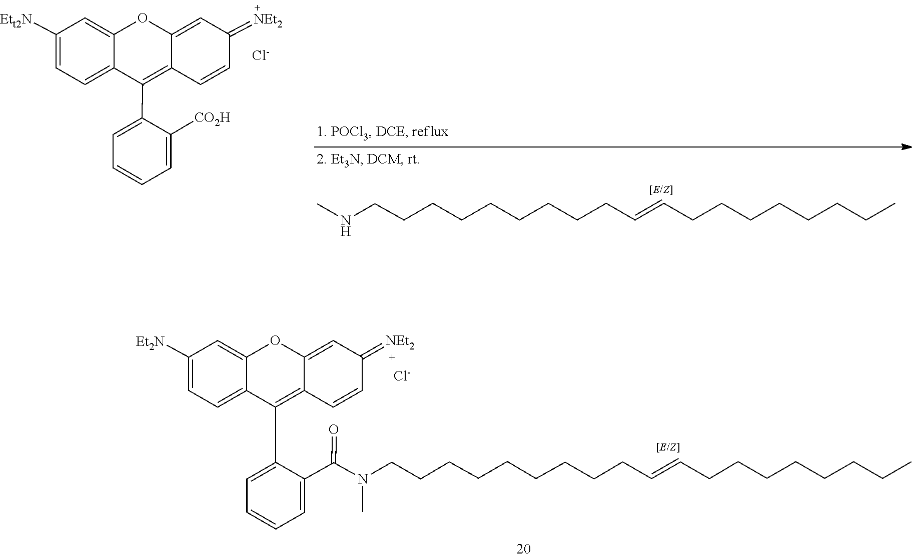

To a 250 mL 1N RBF containing a solution of Rhodamine B (2.00 g, 4.18 mmol, 1.0 eq.) in 1,2-dichloroethane (170 mL) was added POCl₃ (1.40 mL, 15.0 mmol, 3.6 eq.). The bright purple mixture was refluxed for four hours under N₂, then allowed to cool to ambient temperature. The volatiles were removed by rotary evaporation to give the crude acyl halide as a bright purple film that was used without further purification. The residue was dissolved in 100 mL of DCM and an aliquot of 4.5 ml added to a stirred solution of (E/Z)-N-methylnonadec-10-en-1-amine (66 mg, 0.22 mmol, 1.2 eq) in DCM (10 ml). The resultant mixture was stirred for 15 minutes and then Et₃N (77 μL, 0.56 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid.The crude amide was purified by column chromatography eluting with gradient solvent system of DCM/ MeOH (5% DCM/ MeOH- 10% MeOH/DCM), and following rotary evaporation, dried in the vacuum oven (40° C.) overnight to give N-(6-(diethylamino)-9-(2-(methyl(octadec-9-en-1-yl)carbamoyl)phenyl)-3H-xanthen-3-ylidene)-N-ethylethanaminium chloride (20) as a tacky purple solid (630 mg, 75%) . 1H NMR (400 MHz, CDCl₃) δ 7.70-7.60 (m, 2H_{R1}, 2H_{R2}), 7.57-7.53 (m, 1H_{R1}, 1H_{R2}), 7.36-7.30 (m, 1HR1 3 1H_{R2}), 7.30-7.19 (m, 2H_{R1}, 2H_{R2})), 7.08 (d, J=9.6, 2.2 Hz, 2H_{R1}), 6.92 (d, J=2.5 Hz, 2H_{R2}), 6.80 (d, J=2.5 Hz, 2H_{R2}), 5.40-5.30 (m, 2H_{R1}, 2H_{R2}); 3.75-3.42 (m, 8H_{R1}, 8H_{R2}), 3.17 (t, J=7.3 Hz, 2H), 3.05 (t, J=7.7 Hz, 1H); 2.90 (s, 2H), 2.70 (s, 1H); 2.20-1.90 (m, 6H), 1.5-1.00 (m, 38 H), 0.96-0.86 (m, 2H), 0.83 (t, J=6.7 Hz, 3H).NB: Rotamers are denoted R₁/R₂.

### Synthesis of Compound 21

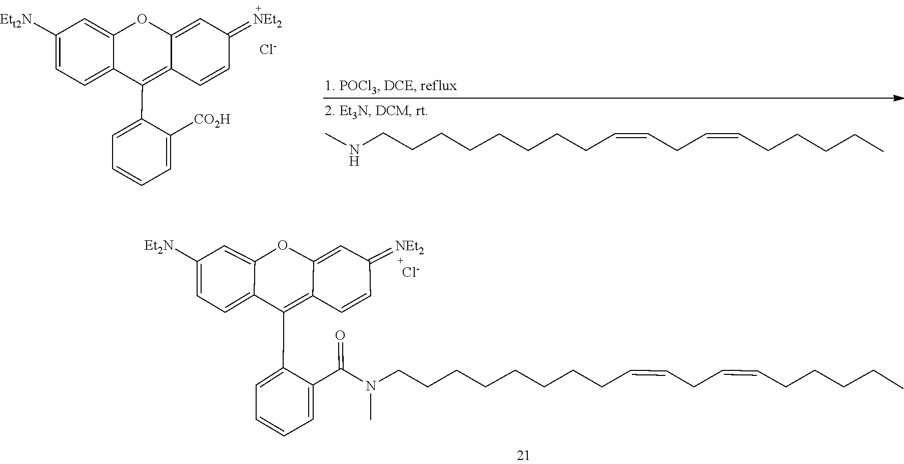

To a 250 mL 1N RBF containing a solution of Rhodamine B (2.00 g, 4.18 mmol, 1.0 eq.) in 1,2-dichloroethane (170 mL) was added POCl₃ (1.40 mL, 15.0 mmol, 3.6 eq.). The bright purple mixture was refluxed for four hours under N₂, then allowed to cool to ambient temperature. The volatiles were removed by rotary evaporation to give the crude acyl halide as a bright purple film that was used without further purification. The residue was dissolved in 100 mL of DCM and an aliquot of 35.8 ml added to a stirred solution of (9Z,12Z)-N-methyloctadeca-9,12-dien-1-amine (0.5 g, 1.79 mmol, 1.2 eq) in DCM (10 ml). The resultant mixture was stirred for 15 minutes and then Et₃N (620 μL, 4.47 mmol, 3.0 eq.) added. The bright purple solution was stirred for 1.5 hours and monitored by TLC. The mixture was quenched with saturated aqueous NaHCO₃ (20 mL) and extracted with DCM (40 mL). The organic phase was washed was H₂O (20 mL), then brine (15 mL), before being dried (Na₂SO₄), passed through a 2 cm plug of silica and washed through with 10% MeOH/DCM (100 mL). Rotary evaporation gave the crude amide as a tacky purple solid. The crude amide was purified by column chromatography eluting with gradient solvent system of DCM/ MeOH (5% DCM/MeOH-10% MeOH/ DCM), and following rotary evaporation, dried in the vacuum oven (40° C.) overnight to give 780 mg of Rhodamine B (9Z,12Z)-N-methyloctadeca-9,12-dien-1-amide (21) as a tacky purple solid (780 mg, 74%). ¹H NMR (400 MHz, CDCl₃) δ 7.67-7.58 (m, 2H_{R1}, 2H_{R2}), 7.53-7.47 (m, 1H_{R1}, 1H_{R2}), 7.36-7.30 (m, 1H_{R1}, 1H_{R2}), 7.26-7.19 (m, 2H_{R1}, 2H_{R2})), 7.02 (dd, J=9.6, 2.2 Hz, 2H_{R1}), 6.87 (d, J=2.5 Hz, 2H_{R2}), 6.84 (d, J=2.5 Hz, 2H_{R2}), 6.74 (d, J=2.3 Hz, 2H_{R1}), 5.40-5.26 (m, 2H_{R1}, 2H_{R2}); 3.75-3.42 (m, 8H_{R1}, 8H_{R2}), 3.13 (t, J=7.3 Hz, 2H), 3.00 (t, J=7.7 Hz, 1H); 2.86 (s, 2H), 2.80-2.68 (m, 2H); 2.02-1.83 (m, 4H), 1.5-1.00 (m, 36 H), 0.96-0.86 (m, 1H), 0.83 (t, J=6.7 Hz, 3H). NB: Rotamers are denoted R₁/R₂.

### Synthesis of Compound 22

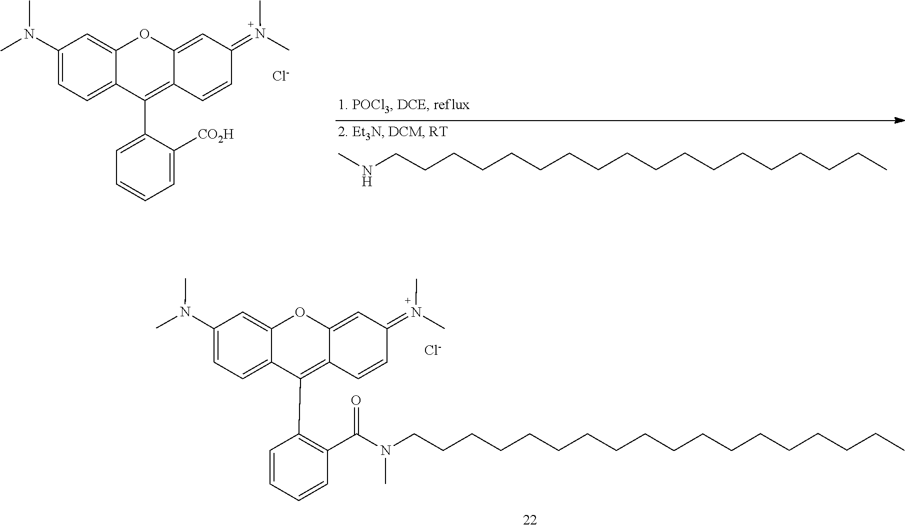

To a 25 mL 1N RBF containing a solution of tetramethylrhodamine chloride (150 mg, 0.355 mmol, 1.0 eq.) in 1,2-dichloroethane (50 mL) was added POCl₃ (196 mg, 120 μL, 1.28 mmol, 3.6 eq.). The bright purple mixture was refluxed for three hours under N₂, then allowed to cool to ambient temperature. The volatiles were removed by rotary evaporation to give the crude acyl chloride as a bright purple film that was used without further purification. The acyl chloride residue was suspended in dry DCM (10 mL) and N-Methyloctadecylamine (121 mg, 0.426 mmol, 1.2 eq.) dissolved in DCM (5 mL) was added to the acyl chloride solution in one portion. The resultant mixture was stirred for 15 minutes and then Et₃N (107 mg, 147 μL, 1.07 mmol, 3.0 eq.) was added. The bright purple solution was stirred for 16 after which time the reaction progress was monitored by TLC. The mixture was quenched with saturated aqueous NaHCO₃ (20 mL) and extracted with DCM (20 mL). The organic phase was washed was H₂O (20 mL), then brine (20 mL) before being dried (MgSO4). Rotary evaporation gave 213 mg of the crude amide as a tacky purple solid. The crude amide was purified by flash column chromatography using a graduated column (3 x 15 cm then 4 x 7 cm silica gel) using an eluent gradient of 5-15% MeOH in DCM. The fractions containing the product were combined and concentrated by rotary evaporation, then dried in the vacuum oven (60° C.) overnight to give tetramethylrhodamine N-methyloctadecylamide (22) as a dark green solid (143 mg, 59%). ¹H NMR (400 MHz, MeOD) δ 7.81-7.71 (m, 2H), 7.68 -7.62 (m, 1H), 7.55-7.49 (m, 1H), 7.32 (dd, J=9.5, 6.2 Hz, 2H), 7.10 (m, 2H), 6.98 (m, 2H), 3.33 (s, 10H), 3.18 (t, J=7.1Hz, 2H), 2.85 (s, 2H), 2.65 (s, 1H), 1.29 (d, J=2.7 Hz, 22H), 1.06 (m, 3H), 0.94-0.87 (m, 3H).

Synthesis of Hexafluoro Rhodamine B

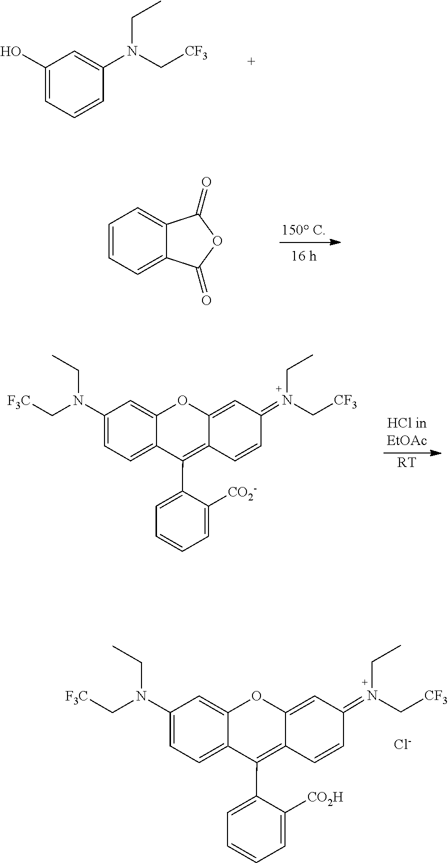

A 25 mL 1N RBF containing a stirrer bar was fitted with an air condenser and charged with N-Ethyl-N-(2,2,2-trifluoroethyl)-3-hydroxyaniline (3.11 g, 14.2 mmol, 2.0 eq.) and phthalic anhydride (1.05 g, 7.10 mmol, 1.0 eq.). The mixture was heated at 160° C. with stirring (500 RPM) for 16 hours to give the crude hexafluoro Rhodamine B free base as a dark purple solid. The crude product was purified by flash column chromatography (6×16 cm column, Note 1) using an eluent gradient of 5-20% MeOH in DCM to give 9-(2-((λ₁-oxidaneyl)carbonyl)phenyl)-3,6-bis(ethyl(2,2,2-trifluoroethyl)amino)xanthylium as a dark red solid (2.19 g). To a 1N 250 mL RBF containing a stir bar and 9-(2-((λ₁-oxidaneyl)carbonyl)phenyl)-3,6-bis(ethyl(2,2,2-trifluoroethyl)amino)xanthylium (2.10 g, 3.81 mmol, 2.24 eq.) in EtOAc (30 mL) was added 2.8 M HCl in EtOAc (10 mL). The reaction mixture was stirred for 5 min and the solvent was then removed by rotary evaporation. The residue was dissolved in DCM (50 mL) and 25 mL of the solution was transferred to a 100 mL separating funnel. The remaining half of the dissolved residue was concentrated by rotary evaporation for later use. The DCM layer was washed with H₂O (3×25 mL) and brine (25 mL) then dried over MgSO4 and filtered on a sintered glass funnel. The filtrate was then concentrated by rotary evaporation to give hexafluoro rhodamine B (1.00 g) as a glass-like dark red solid. The other half of the concentrated reaction mixture residue was re-dissolved in 50 mL of DCM and 25 mL was transferred to a 100 mL separating funnel. The DCM layer was washed with H₂O (3×25 mL) and brine (25 mL) then dried over MgSO4 and filtered on a sintered glass funnel. The filtrate was then concentrated by rotary evaporation to give hexafluoro rhodamine B (579 mg) as a dark red solid. The remaining one-quarter of the reaction mixture residue in 25 mL of DCM was transferred to a 100 mL separating funnel. The DCM layer was washed with H₂O (3×25 mL) and brine (25 mL) then dried over MgSO4 and filtered on a sintered glass funnel. The filtrate was then concentrated by rotary evaporation to give hexafluoro rhodamine B (613 mg) as a dark red solid. Total overall yield: 2.19 g, 96%. ¹H NMR (400 MHz, CDCl₃) δ 8.05-7.96 (m, 1H), 7.71 — 7.56 (m, 2H), 7.23-7.17 (m, 1H), 7.09 (t, J=8.1Hz, 1H), 6.67-6.59 (m, 4H), 6.45 (dd, J =8.9, 2.7 Hz, 2H), 6.39-6.23 (m, 2H), 3.91-3.72 (m, 6H), 3.56-3.35 (m, 6H), 1.28-1.08 (m, 9H).

### Synthesis of Compound 23

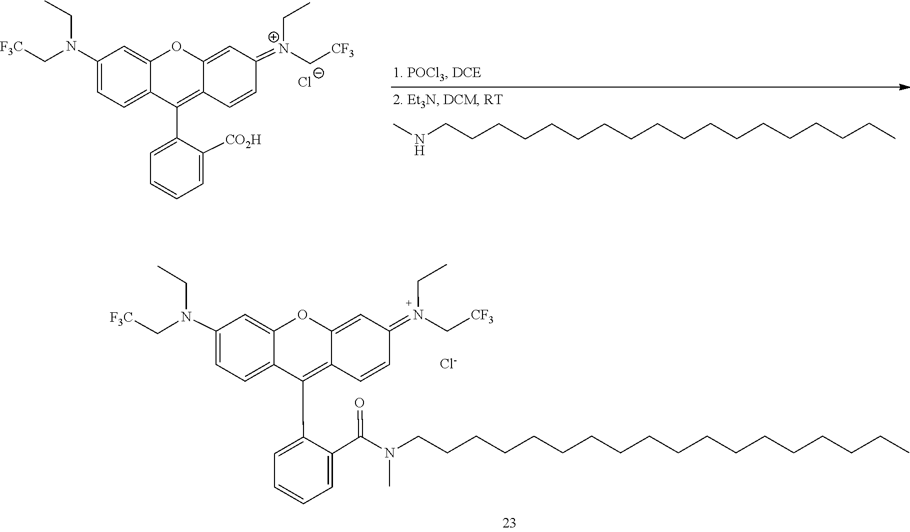 [SMILES: CCN(CC(F)(F)F)c1ccc2c(-c3ccccc3C(=O)O)c3cc/c(=[N+](/CC)CC(F)(F)F)cc-3oc2c1 ; [Cl-] ; CCCCCCCCCCCCCCCCCCNC ; CCCCCCCCCCCCCCCCCCN(C)C(=O)c1ccccc1-c1c2cc/c(=[N+](/CC)CC(F)(F)F)cc-2oc2cc(N(CC)CC(F)(F)F)ccc12 ; [Cl-]]

To a 250 mL 1N RBF containing a solution of hexafluoro rhodamine B (579 mg, 0.986 mmol, 2.0 eq.) in 1,2-dichloroethane (50 mL) was added POCl₃ (544 mg, 332 μL, 3.55 mmol, 7.2 eq.). The bright purple mixture was refluxed for four hours under N₂, then allowed to cool to ambient temperature. The volatiles were removed by rotary evaporation to give the crude acyl chloride as a bright purple film that was used without further purification. The acyl chloride residue was suspended in dry DCM (40 mL) and a 20 mL portion (1.0 eq. of acyl chloride) was taken from the resultant solution. N-methyloctadecylamine (168 mg, 0.592 mmol, 1.2 eq.) dissolved in DCM (5 mL) was added to the 20 mL of acyl chloride solution in one portion. The resultant mixture was stirred for 15 minutes and then Et₃N (150 mg, 205 μL, 1.48 mmol, 3.0 eq.) was added. The bright purple solution was stirred for 16 h after which time the reaction progress was monitored by TLC. The mixture was quenched with saturated aqueous NaHCO3 (25 mL) and extracted with DCM (25 mL). The organic phase was washed was H₂O (25 mL), then brine (25 mL) before being dried (MgSO4). Rotary evaporation gave 477 mg of the crude amide as a tacky purple solid. The crude amide was purified by flash column chromatography (4×20 cm silica gel) using an eluent gradient of 5-15% MeOH in DCM. The fractions containing the product were combined and concentrated by rotary evaporation, then dried in the vacuum oven (60° C.) overnight to give hexafluoro Rhodamine B N-methyloctadecylamide (23) as a dark red solid (242 mg, 58%). 1H NMR (400 MHz, CDCl₃) δ 7.72-7.62 (m, 2H), 7.49-7.33 (m, 5H), 7.22-7.16 (m, 1H), 7.06 (d, J=2.2 Hz, 1H), 4.43 (q, J=8.6 Hz, 4H), 3.89-3.75 (m, 4H), 3.18-3.06 (m, 2H), 2.99 (s, 2H), 2.68 (s, 1H), 2.21-1.94 (m, 3H), 2.09 (s, 4H), 1.37 (t, J=7.0 Hz, 6H), 1.30-1.02 (m, 34H), 0.96 (d, J=6.6 Hz, 2H), 0.91-0.82 (m, 4H).

### Synthesis of Compound 24

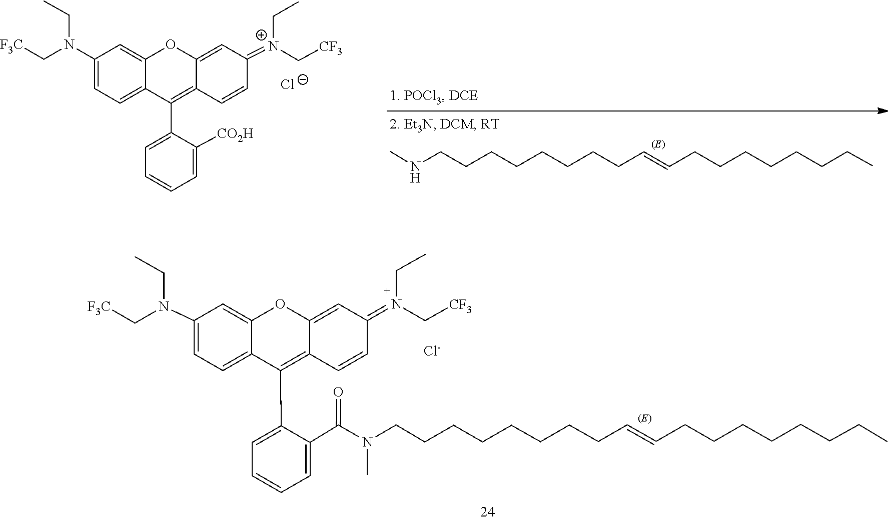 [SMILES: CCN(CC(F)(F)F)c1ccc2c(-c3ccccc3C(=O)O)c3cc/c(=[N+](/CC)CC(F)(F)F)cc-3oc2c1 ; [Cl-] ; CCCCCCCC/C=C/CCCCCCCCNC ; CCCCCCCC/C=C/CCCCCCCCN(C)C(=O)c1ccccc1-c1c2cc/c(=[N+](/CC)CC(F)(F)F)cc-2oc2cc(N(CC)CC(F)(F)F)ccc12 ; [Cl-]]

To a 250 mL 1N RBF containing a solution of hexafluoro rhodamine B (579 mg, 0.986 mmol, 2.0 eq.) in 1,2-dichloroethane (50 mL) was added POCl₃ (544 mg, 332 μL, 3.55 mmol, 7.2 eq.). The bright purple mixture was refluxed for four hours under N₂, then allowed to cool to ambient temperature. The volatiles were removed by rotary evaporation to give the crude acyl chloride as a bright purple film that was used without further purification. The acyl chloride residue was suspended in dry DCM (40 mL) and a 20 mL portion (1.0 eq. of acyl chloride) was taken from the resultant solution. (E)-N-Methyloctadec-9-en-1-amine (167 mg, 0.592 mmol, 1.2 eq.) dissolved in DCM (5 mL) was added to the 20 mL of acyl chloride solution in one portion. The resultant mixture was stirred for 15 minutes and then Et₃N (150 mg, 205 μL, 1.48 mmol, 3.0 eq.) was added. The bright purple solution was stirred for 16 h after which time the reaction progress was monitored by TLC. The mixture was quenched with saturated aqueous NaHCO₃ (25 mL) and extracted with DCM (25 mL). The organic phase was washed was H₂O (25 mL), then brine (25 mL) before being dried (MgSO₄). Rotary evaporation gave 418 mg of the crude amide as a tacky purple solid. The crude amide was purified by flash column chromatography (4×20 cm silica gel) using an eluent gradient of 5-15% MeOH in DCM. The fractions containing the product were combined and concentrated by rotary evaporation, then dried in the vacuum oven (60° C.) overnight to give hexafluoro Rhodamine B N-methyloctadecylamide (24) as a dark red solid (254 mg, 61%). ¹H NMR (400 MHz, CDCl₃) δ 7.71-7.63 (m, 2H), 7.55 (dd, J=7.0, 2.0 Hz, 1H), 7.48-7.35 (m, 4H), 7.19 (d, J=8.9 Hz, 1H), 7.05 (d, J=2.3 Hz, 1H), 5.46-5.24 (m, 2H), 4.42 (q, J=8.3, 7.9 Hz, 4H), 3.91-3.75 (m, 4H), 3.13 (t, J=7.5 Hz, 1H), 2.99 (s, 2H), 2.68 (s, 1H), 2.21-1.88 (m, 4H), 1.43-1.02 (m, 29H), 1.00-0.92 (m, 1H), 0.90 -0.81 (m, 3H).

### Synthesis of Compound 25

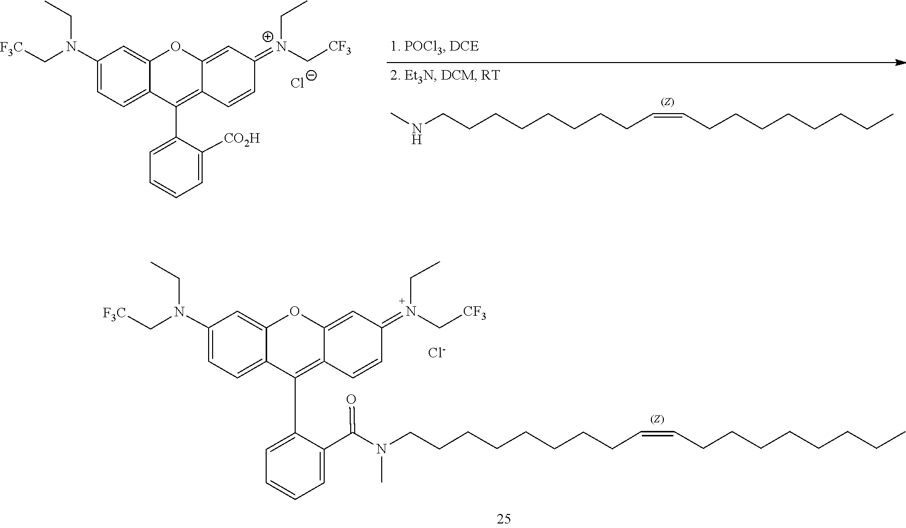

To a 250 mL 1N RBF containing a solution of hexafluoro rhodamine B (1.00 g, 1.70 mmol, 1.0 eq.) in 1,2-dichloroethane (100 mL) was added POCl₃ (938 mg, 572 μL, 6.12 mmol, 3.6 eq.). The bright purple mixture was refluxed for four hours under N₂, then allowed to cool to ambient temperature. The volatiles were removed by rotary evaporation to give the crude acyl chloride as a bright purple film that was used without further purification. The acyl chloride residue was suspended in dry DCM (80 mL) and (Z)-N-methyloctadec-9-en-1-amine (574 mg, 2.04 mmol, 1.2 eq.) dissolved in DCM (20 mL) was added in one portion. The resultant mixture was stirred for 15 minutes and then Et₃N (516 mg, 707 μL, 5.10 mmol, 3.0 eq.) was added. The bright purple solution was stirred for 1.5 hours and monitored by TLC. The mixture was quenched with saturated aqueous NaHCO₃ (100 mL) and extracted with DCM (200 mL). The organic phase was washed was H₂O (100 mL), then brine (75 mL) before being dried (Na₂SO₄). Rotary evaporation gave 1.78 g of the crude amide as a tacky purple solid. The crude amide was purified by flash column chromatography (4×20 cm silica gel) using an eluent gradient of 5-15% MeOH in DCM. The fractions containing the product were combined and concentrated by rotary evaporation, then dried in the vacuum oven (60 ° C.) overnight to give hexafluoro rhodamine B N-methyloleylamide (25) as a dark red solid (478 mg, 33%). ¹H NMR (400 MHz, CDCl₃) δ 7.73-7.61 (m, 2H), 7.55 (dd, J=6.6, 2.3 Hz, 1H), 7.49-7.32 (m, 4H), 7.24-7.15 (m, 1H), 7.05 (d, J=2.4 Hz, 1H), 5.40-5.26 (m, 2H), 4.43 (p, J=8.3 Hz, 3H), 3.83 (h, J=7.6 Hz, 4H), 3.13 (q, J=6.3, 5.1 Hz, 2H), 3.00 (d, J=2.0 Hz, 2H), 2.68 (s, 1H), 1.98 (p, J=6.8 Hz, 4H), 1.37 (t, J=7.1 Hz, 6H), 1.31-1.23 (m, 15H), 1.10 (s, 3H), 0.91-0.83 (m, 3H).

### Example 2. Spectral Scan of Dyes

A spectral scan was conducted for dyes 1-25 according to the following procedure. 200 μL of 2 μM dye was pipetted into a Costar 96 well flat bottom plate. The plate was scanned on a CLARIOStar spectrophotometer. Each well was scanned at a single point. For the absorption scan, the expected emission wavelength was set at 598 nm, and the absorption data was collected from 450-568 nm. For the emission spectra, the expected absorption wavelength was set to 552 nm and the emission spectra was collected from 580-700 nm. The gain was set at 1000.

The maxima of absorption for the Rhodamine family was determined to be 564 nm and maxima for emission was determined to be 588 nm. It is noted most systems have a bandwidth of about 10-20 nm. For subsequent experiments, fluorescence was set at 562 nm/583 nm (Ex/Em).

### Example 3. Labelling of Extracellular Vesicles with Dyes

Labelling of extracellular vesicles (EVs) with dyes 1-10 was performed according to the following procedure. 20 μL of platelet-derived EVs were incubated in HPLC vials with or without each dye at a final concentration of 2 μM dye, the dye stock solution being made up in water. For negative controls, an equivalent volume of the diluent solution was added to the EV solution. The sample was mixed gently. Each sample was subsequently incubated for 1 hr at 37° C. Samples were transferred and stored in the autosampler set at 4° C. A batch table was setup that injected unlabelled EVs prior to labelled EVs. A blank injection was run after each labelled EV experiment to ensure there was no carryover.

15 μL of each sample was injected onto a Superose 6 Increase column (3.2/300) from Cytiva. The flow rate was set at 0.15 mL min* The Shimadzu BioHPLC was run in isocratic mode with Buffer A: 1× PBS (phosphate-buffered saline), pH 7.4, 100 nm filtered. The column oven temperature was set at 30° C. Total run duration was 30 min. Data was collected using two detectors. The PDA (UV detector) was set to scan from 190-800 nm and a channel of the FLD (fluorescence detector) was set at 562 nm/583 nm (Ex/Em). Titration experiments were conducted to confirm that excess dye was removed. The areas of the void peaks from the 214 nm absorption and the 583 nm emission were calculated by the LabSolutions software using the manual integration function. To normalise the fluorescent signal, the total fluorescent area was divided by the UV 214 nm area. This ratio can be used to rank each dye, with higher ratios indicative of greater incorporation.

The results are summarised in Table 1. All dyes 1-10 were found to incorporate into EVs, with each sample containing EV+dye exhibiting a higher FLD/UV ratio compared to the respective EV alone samples. These results indicate that dyes 1-10 are capable of being incorporated into EVs.

TABLE 1
UV and FLD data of EVs with and without dye

| | EV + dye | | | EV alone | | |
|---|---|---|---|---|---|---|
| | | | Area FLD/ | | | Area FLD/ |
| Dye | UV area | FLD area | Area UV | UV area | FLD area | Area UV |
| 1 | 1092869 | 2281007 | 2.09 | 1128078 | 896318 | 0.79 |
| 2 | 554454 | 2493643 | 4.50 | 766929 | 624693 | 0.81 |
| 3 | 1177448 | 4270562 | 3.63 | 1307744 | 1087337 | 0.83 |
| 4 | 913937 | 1901680 | 2.08 | 970922 | 815649 | 0.84 |
| 5 | 1918454 | 2405326 | 1.25 | 2101807 | 1837760 | 0.87 |
| 6 | 1803632 | 4247970 | 2.36 | 1857512 | 1607151 | 0.87 |
| 7 | 1056319 | 1456410 | 1.38 | 1387348 | 1152890 | 0.83 |
| 8 | 1173725 | 3422473 | 2.92 | 1535405 | 1237838 | 0.81 |
| 9 | 1367007 | 4558422 | 3.33 | 1589460 | 1332220 | 0.84 |
| 10 | 1106893 | 2826617 | 2.55 | 1119328 | 952255 | 0.85 |

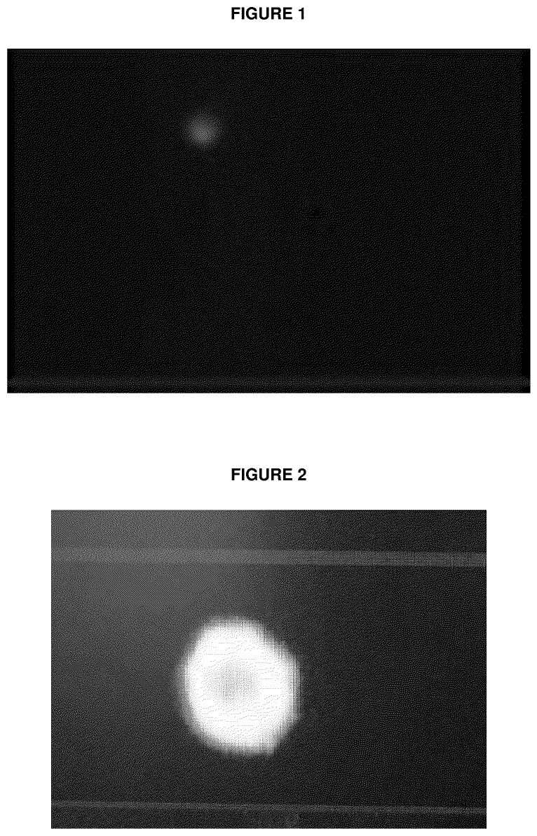

Flow cytometry also confirmed incorporation of the dyes into EVs. FIG. 1 shows the detection of EV+dye 2 using the Amnis ImageStreamX MKI I imaging flow cytometer. This result indicates that the dyes are capable of labelling EVs.

### Example 4. Labelling of Human Cells with Dyes

Labelling of human cells with dyes 1-10 was performed according to the following procedure. Each well of a Costar 96 well flat bottom plate was seeded with 5,000 normal human dermal fibroblasts (NHDF; not older than P9) and allowed to attach overnight in full media: fibroblast growth media (Lonza)+2% v/v fetal calf serum. Each dye was diluted 1:50 from a stock of 1 mM with 10 mM L-histidine, 150 mM NaCl, pH 6.0, to a final concentration of 20 μM. The dye was subsequently added to full media at a further 1:10 dilution to a final concentration of 2 μM. The media on the cells was subsequently replaced with the freshly prepared full media containing the dye. For negative controls, an equivalent volume of the diluent solution was added to the cell solution. Cells were incubated for 1 hr at 37° C. and then washed with 1 xHBSS (Hank's balanced salt solution) to remove free dye. Plate was imaged using the CLARIOStar spectrophotometer (5x5 matrix mode, average of 25 data points, 3 wells for each dye). A higher fluorescence indicates a higher amount of incorporation into the cells.

The results are summarised in Table 2. All dyes 1-10 were found to incorporate into NHDFs, with each sample containing dye exhibiting a higher FLD compared to NHDFs alone. These results indicate that dyes 1-10 are capable of being incorporated human cells.

TABLE 2
FLD data of NHDFs with and without dye

| Dye | FLD |
|---|---|
| 1 | 120641 |
| 2 | 123449 |
| 3 | 43073 |
| 4 | 18027 |
| 5 | 106332 |
| 6 | 20270 |
| 7 | 97347 |
| 8 | 83404 |
| 9 | 83536 |
| 10 | 45943 |
| No dye | 1262 |

Flow cytometry also confirmed incorporation of the dyes into the NHDFs. FIG. 2 shows the detection of Cell+dye 2 using the Amnis ImageStreamX MKII imaging flow cytometer. This result indicates that the dyes are capable of labelling human cells such as NHDFs.

### Example 5. Imaging Human Cells Labelled with Dye

The cells were imaged to assess incorporation of the dyes according to the following procedure. Cell+dye 2 samples were prepared following the procedure in Example 4 with the following modifications: the cells were seeded with NHDF (not older than P9), human umbilical cord-derived mesenchymal stem cells (UC-MSCs) or bone marrow-derived MSCs (BM-MSCs); and the dye 2 was used in a final concentration of 0.2 μM. Before imaging, each well was washed with 1× HBSS (−) once. 100-200 μL of 1× HBSS (−) was added to each well prior to imaging to make sure the cells are not dried out. The cells were imaged on a Thermo Fisher Evos M5000 microscope equipped with the Texas Red filter. Routinely utilized conditions are (magnification, light, exposure, gain): 10× Texas Red, 22, 146, 105; 20× Texas Red, 12, 133, 64; 40× Texas Red, 36, 79, 40. Settings were held constant for all images taken on the same plate.

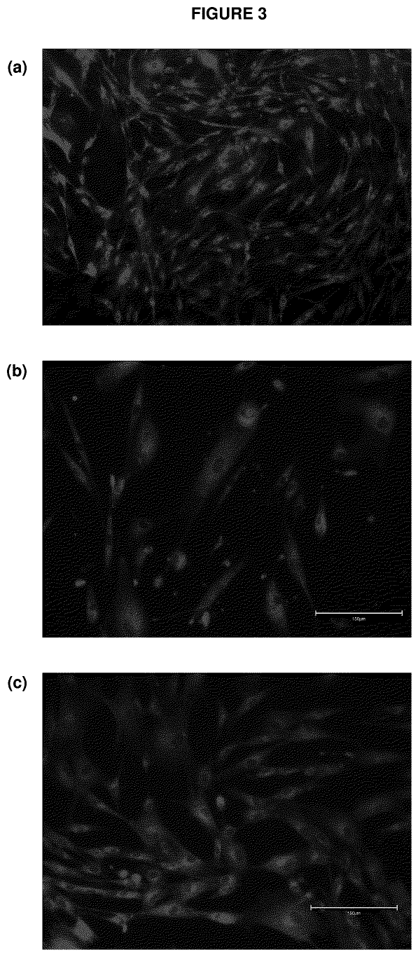

The microscopy images are shown in FIG. 3 and demonstrate that dye 2 is capable of labelling the NHDF, UC-MSCs and BM-MSCs (FIGS. 3(a), (b) and (c), respectively). The results indicate that the dyes are capable of labelling various human cells.

### Example 6. Procedure for Removing Excess Dye

The capacity of columns to remove excess dye was investigated according to the following procedure. ZebaTM Spin Desalting Columns, 40K Molecular Weight Cut Off (MWCO), 2 mL from ThermoFisher Scientific were used to remove excess dye. Centrifugation was performed following the instructions provided by ThermoFisher for using a 40 kDa cut-off Zeba spin desalting column. Dye 2 was assessed at 3 concentrations (2 μM, 0.2 μM and 0.02 μM) for its capacity to pass through a 40 kDa cut-off Zeba spin desalting column. A control well contained NHDF cells only. The rationale behind this procedure was to determine, if x% of dye 2 was “free” after incubation with EVs/cells, could the Zeba column be used to remove the “free” (excess) dye: the 2 μM, 0.2 μM and 0.02 μM samples represent 100%, 10% and 1% “free” dye 2, respectively. The samples were analysed using the CLARIOStar spectrophotometer (5×5 matrix mode, average of 25 data points, 3 wells for each dye).

The results are shown in Table 3. The results show about 15% of the dye passed through for the 2 μM, whereas essentially none of the dye passed through for the 0.2 μM and 0.02 μM samples. The results indicate that excess dye is capable of being removed using a suitable column, with the amount of dye passing through the column unlikely to affect experimental results.

TABLE 3
FLD data of dye with and without passing through Zeba column

| | Fluorescence - | Fluorescence - |
|---|---|---|
| Sample | no Zeba column | Zeba column |
| 2 μM dye 2 | 10299 ± 132 | 1529 ± 14 |
| 0.2 μM dye 2 | 1829 ± 32 | 1206 ± 20 |
| 0.02 μM dye 2 | 1130 ± 20 | 1151 ± 14 |
| Cells only | 1111 ± 26 | — |

### Example 7. Labelling of Human Cells with Dyes and Labelled Extracellular Vesicles

Labelling of NHDF cells with dyes 11-18 alone and EVs labelled with dyes 11-18 was performed according to the following procedure. Each well of a Costar 96 well flat bottom plate was seeded with 5,000 normal human dermal fibroblasts (NHDF; not older than P9) and allowed to attach overnight in full media: fibroblast growth media (Lonza)+2% v/v fetal calf serum. Each dye was diluted in water to a final concentration of 1 mM. The dyes were then diluted to a final concentration of 2 μM in either a base formulation (1× PBS) or a sample of Plexaris (purified EVs derived from platelets) in 1× PBS and incubated at 1 hr at 37° C. The dyes were subsequently added onto the NHDFs at a further 1:10 dilution to a final concentration of 0.2 μM. Cells were incubated for 1 hr at 37° C. and then washed with 1× HBSS (Hank's balanced salt solution) to remove free dye. Plate was imaged using the CLARIOStar spectrophotometer (5×5 matrix mode, average of 25 data points, 3 wells for each dye). The data was normalised against either dye 2 or Plexaris+dye 2. A higher fluorescence indicates a higher amount of incorporation into the cells.

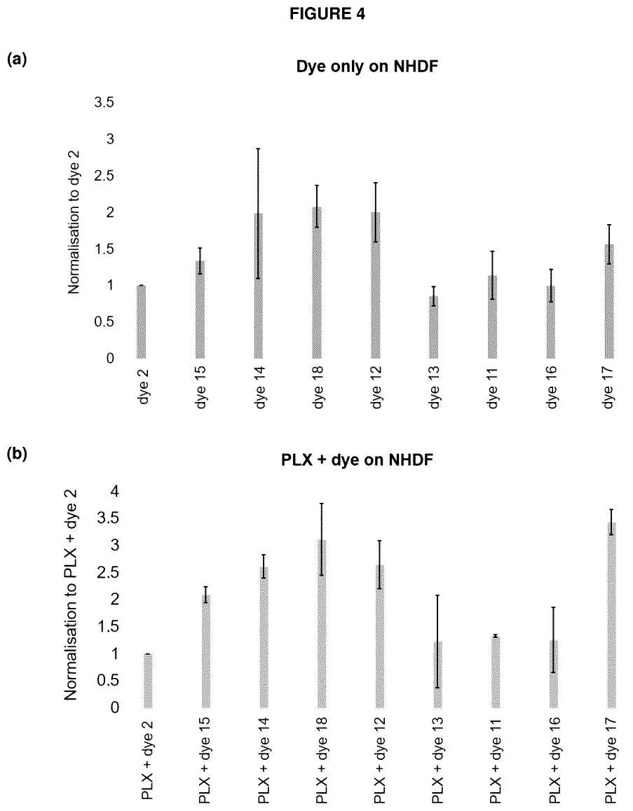

FIG. 4(a) shows the results of dye only on NHDFs and FIG. 4(b) shows the results of Plexaris+dye on NHDFs. All dyes 11-18, alone or with Plexaris (PLX), were found to incorporate into NHDFs, indicating that dyes 11-18 and EVs tagged with dyes 11-18 are capable of being incorporated human cells. The results also provide an indication that dyes 12, 14 ,15, 17 and 18, alone or with PLX, were incorporated to the cells to a larger extent than dye 2 alone and PLX+dye 2 respectively.

### Example 8. Procedure for Removing Excess Dye

To explore whether dyes 17 and 18 could be removed using a Zeba™ Spin Desalting Columns, 40K Molecular Weight Cut Off (MWCO), the dyes were prepared a 2 μM concentration in base formulation (1× PBS) and spun through a Zeba spin column. Removal of the dyes was compared to Plexaris+dye 2, with Plexaris alone and NHDF cells alone used as controls.

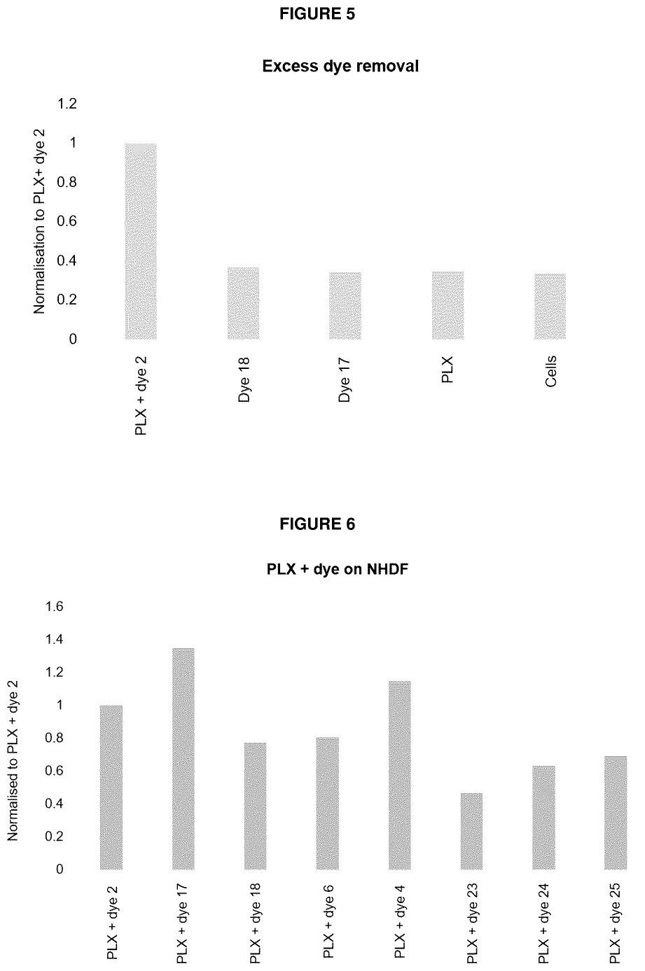

The results are shown in FIG. 5, in which the data is normalised to PLX+dye 2. The results demonstrate that no cell staining was observed for dyes 17 and 18. The results indicate that excess dye is capable of being removed using a suitable column, with the amount of dye passing through the column unlikely to affect experimental results.

Example 9. Labelling of human cells with labelled extracellular vesicles

Labelling of NHDF cells with EVs labelled with dyes 4, 6, 17, 18, 23, 24 and 25 was performed according to the following procedure. Each well of a Costar 96 well flat bottom plate was seeded with 5,000 normal human dermal fibroblasts (NHDF; not older than P9) and allowed to attach overnight in full media: fibroblast growth media (Lonza)+2% v/v fetal calf serum. Each dye was diluted in phosphate buffered saline (PBS) to a final concentration of 1 mM. The dyes were then diluted to a final concentration of 2 μM in Plexaris (purified EVs derived from platelets) in PBS and incubated at 1 hr at 37° C. The dyes were subsequently added onto the NHDFs at a further 1:10 dilution to a final concentration of 0.2 μM. Cells were incubated for 1 hr at 37° C. and then washed with 1× HBSS (Hank's balanced salt solution) to remove free dye. Plate was imaged using the CLARIOStar spectrophotometer (5×5 matrix mode, average of 25 data points, 3 wells for each dye). The data was normalised against Plexaris+dye 2. A higher fluorescence indicates a higher amount of incorporation into the cells.

The results are shown in FIG. 6. All dyes 4, 6, 17, 18, 23, 24 and 25 with Plexaris (PLX) were found to incorporate into NHDFs, indicating that EVs labelled with dyes 4, 6, 17, 18, 23, 24 and 25 are capable of being incorporated human cells.

### Example 10. Cell Proliferation Assay Using Labelled Extracellular Vesicles

To investigate whether the dyes had an effect on cell proliferation, EVs labelled with dyes of the invention were investigated according to the following procedure. The wells of a Costar 96 well flat bottom plate were seeded with 5,000 normal human dermal fibroblasts (NHDF; not older than P9) and allowed to attach overnight in 200 μL complete media: fibroblast growth media (Lonza)+2% v/v fetal calf serum. After 24 hours, the complete media was removed without disturbing the adhered cells at the bottom of the well and each well was washed once with 200 μL HBSS (Hank's balanced salt solution). 200 μL of basal media (fibroblast growth media (Lonza) +0.1% v/v fetal calf serum) was added to each well. To prepare the labelled EVs, 7 mM stock solutions of dyes 2, 4, 6, 17, 18, 23, 24 and 25 in ethanol were diluted 1:350 in PBS to provide a final concentration of 20 μM. The dyes were subsequently added to HEK EVs (1× PBS) at a further 1:10 dilution and incubated at 1 hr at 37° C. A sample of unlabelled Plexaris was also prepared by diluting 360 μL Plexaris with 40 μL PBS.

After 16 hours, the basal media was removed from the experimental wells and 200 μL per well of treatment conditions and control were added (n =3 for each). The assay was stopped after 72 hours. The delta cell index was used for data analysis, using normalisation time (first reading after treatments were added). Data was analysed at 24, 36, 48 and 72 hours post-treatment, normalising the values to the respective basal media for each time point. GraphPad Prism (version 8) was used to perform the Kruskal-Wallis nonparametric one-way analysis of variance, using multiple comparisons (uncorrected Dunn's test).

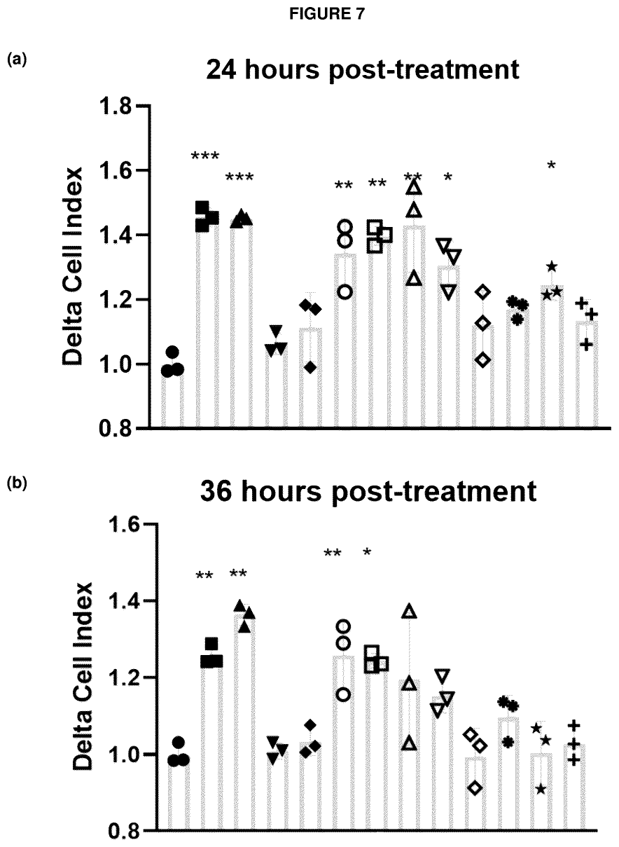

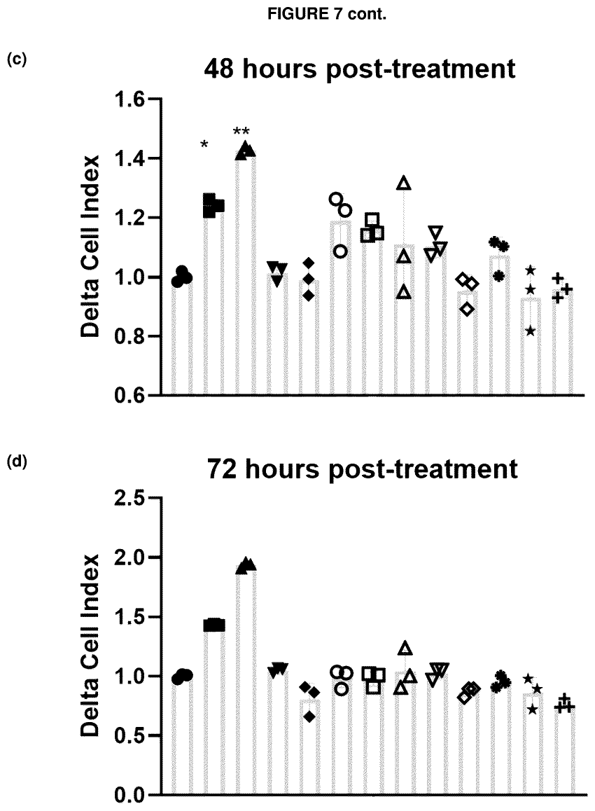

The results at 24, 36, 48 and 72 hours post-treatment are shown in FIGS. 7(a)-7(d) respectively. The samples are represented as follows: 0.1% basal media (fibroblast growth media (Lonza)+0.1% v/v fetal calf serum): filled circles; 2% complete media (fibroblast growth media (Lonza)+2% v/v fetal calf serum): filled squares; 10% high media (fibroblast growth media (Lonza)+10% v/v fetal calf serum): filled upward triangles; PBS control in 0.1% basal media: filled downward triangles; PLX +dye 23: filled diamonds; PLX+dye 24: unfilled circles; PLX+dye 25: unfilled squares; PLX+dye 18: unfilled upward triangles; PLX+dye 17: unfilled downward triangles; PLX+dye 4: unfilled diamonds; PLX+dye 6: asterisks; PLX+dye 2: stars; PLX alone: pluses. Significance is represented as follows: (*) denotes <0.05; (**) denotes <0.005; (***) denotes <0.0005; non-significance is denoted by the lack of (*). The results indicate that the dyes had little effect on cell proliferation compared to 0.1% basal media over the 72 hour time period.

### Example 11. Immunomodulatory Assay Using Labelled Extracellular Vesicles

In this study, the labelling efficiency of EV preparations from mesenchymal stromal cells (MSC-EVs) with dye 2 of the invention as well as conventionally used dyes BODIPY-TR-CER, Calcein AM, CFSE and PKH67 was investigated. Counterstaining of PKH67 and dye 2-labelled objects was performed with anti-tetraspanin antibodies. The impacts of dye 2 labelling on the immunomodulatory capabilities of the MSC-EV preparation were investigated in a multi-donor mixed lymphocyte reaction (mdMLR) assay. Furthermore, the uptake of dye-2 labelled objects by the different immune cells within the mdMLR assay were evaluated.

### Materials and Methods

MSC-EVs were prepared form human platelet lysate containing MSC-conditioned media by polyethylene glycol 6000 precipitation followed by ultracentrifugation, as described previously (Borger, V. et al, 2020, Scaled Isolation of Mesenchymal Stem/Stromal Cell-Derived Extracellular Vesicles. Curr Protoc Stem Cell Biol 55, e128; Kordelas et al., 2014, MSC-derived exosomes: a novel tool to treat therapy-refractory graft-versus-host disease. Leukemia 28, 970-973; Ludwig et al., 2018, Precipitation with polyethylene glycol followed by washing and pelleting by ultracentrifugation enriches extracellular vesicles from tissue culture supernatants in small and large scales. J Extracell Vesicles 7, 1528109). Conditioned media were harvested every 48 h. Obtained MSC-EV preparations were diluted in NaCl-HEPES buffer (Sigma-Aldrich, Taufkirchen, Germany) such that 1 mL of final samples contained the preparation yield of the conditioned media of approximately 4.0×10⁷ cells.

### Characterisation of EV Preparations

Obtained EV preparations were characterized according to the MISEV criteria (Thery et al., 2018, Minimal information for studies of extracellular vesicles 2018 (MISEV2018): a position statement of the International Society for Extracellular Vesicles and update of the MISEV2014 guidelines. J Extracell Vesicles 7, 1535750.). Briefly, average particle concentrations were determined by NTA on a ZetaView PMX-120 platform equipped with the software version 8.03.08.02 (ParticleMetrix, Meerbusch, Germany) as described previously (Ludwig et al., 2018). Protein concentration was determined by bicinchoninic acid (BCA) assay (Pierce, Rockford, IL, USA) in 96-well plates according to the manufacturer's recommendations. The presence of EV specific proteins (CD9, CD63, CD81 and Syntenin) and the absence of impurities (Calnexin) were confirmed in Western Blots performed as described previously (Ludwig et al., 2018).

EV labelling with Dyes

The staining with CFSE (Thermo Fisher Scientific, Darmstadt, Germany) was based on the manufacturer's protocol. Slight modifications were required to reduce the signal from unbound CFSE. Briefly, the CFSE stock solution was diluted to a working solution of 10 μM CFSE. The solution was centrifuged three times for 10 min at 17,000 × g. Subsequently, 25 μL of MSC-EV preparations, corresponding to the amount of EVs derived from 1×10⁶ MSCs, were incubated with the centrifuged CFSE solution for 20 min at 37° C. The sample was diluted 1:20 to a final volume of 1 mL prior to analysis.

The staining of MSC-EV preparations with Calcein AM followed the manufacturer's instructions. Briefly, 25 μL of MSC-EV preparations were incubated with 25 μL of a 20 μM solution of Calcein AM (Thermo Fisher Scientific) for 40 min at 37° C. The sample was diluted 1:20 to a final volume of 1 mL to reduce background noise, avoiding the requirement of a washing step.

The staining of MSC-EV preparations with BODIPY-TR-Ceramide followed the manufacturer's instructions. Briefly, 25 μL of the MSC-EV preparation, corresponding to EVs purified from 4×10 6 MSCs, were incubated with 25 μL of a 20 μM solution of BODIPY TR Ceramide (Thermo Fisher Scientific) for 20 min at 37° C. 450 μL of 0.9% NaCl with 10mM HEPES (0.9% NaCl, Melsungen, B. Braun; HEPES, Thermo Fisher Scientific) buffer was added and the EVs were washed by using a Centrifugal Concentrator (Vivaspin 500; Sartorius, Gottingen, Germany). The retentate was adjusted to 500 μL prior to analysis.

The staining of MSC-EV preparations with PKH67 followed the manufacturer's instructions for labeling EVs (Thermo Fisher Scientific). Briefly, using 200 μL of given MSC-EV preparations, corresponding to EVs purified from 8×10⁶ cells, the solution was adjusted with Diluent C to a final volume of 1 mL. 6 μL of PKH67 dye was added to each tube and mixed continuously for 30 seconds. After 5 min at room temperature, the solution was quenched by adding 2 mL of 10% (w/v) bovine serum albumin fraction 5 (Carl Roth, Karlsruhe, Germany). Serum-free medium, DMEM low glucose (PAN Biotech, Aidenbach, Germany) supplemented with 100 U/ mL penicillin-streptomycin-glutamine (Thermo Fisher Scientific, Darmstadt, Germany), was used to adjust the volume to 8.5 mL. 1.5 mL of a 0.971 M sucrose solution (Carl Roth) was added to the bottom of the tube, and the tube was centrifuged for 2 hours at 190,000× g in a swing-out rotor (SW40 Ti; 159 Beckman Coulter, Krefeld, Germany; k-factor: 137) at 4° C. The supernatant was discarded, and the pellet resuspended in Na-HEPES buffer. After resuspension, the volume was adjusted to 5 mL and transferred to a Centrifugal Concentrator (Vivaspin 6; Sartorius). The retentate was adjusted to 120 μL prior to analysis.

The MSC-EV preparations were stained with dye 2 of the invention according to the following protocol. Dye 2 was provided as a lyophilised powder. 1 mg of dye 2 was resuspended with 1 mL buffer to a final concentration of 0.2 μM. Like CFDA-SE, the dye 2 solution was centrifuged for 10 min at 17,000 x g to reduce background noise to a minimum. Briefly, for the EV-labelling 25 μL of the MSC-EV preparations were incubated with 25 μL of a prepared, centrifuged dye 2 solution (0.2 μM) for 1 hour at 37° C. The sample was diluted 1:20 to a final volume of 1 mL prior to analysis. For EV uptake experiments dye 2-labelled MSC-EV preparations were cleared from EV unbound dye 2 by ultrafiltration. Briefly, after labelling with dye 2, the MSC-EVs were washed by via centrifugation at 12,000xg through Vivaspin 500 filters (Sartorius) for 10 min. The retentate was collected as labelled EV sample.

### Antibody Labelling of PREPARED EVS

After dye labelling, 5 μL of dye 2-stained MSC-EV samples were mixed with 20 μL of a 10 nM anti-human CD9 FITC (EXBIO, Vestec, Czech Republic), 12 nM anti-human CD63 AF488 (EXBIO) or 13 nM anti-human CD81 FITC (Beckman Coulter) antibody solution, respectively. For PKH67 stained MSC-EV samples, incubated with 10 nM anti-human CD9 PE (EXBIO), 12 nM anti-human CD63 PE (EXBIO) or 13 nM anti-human CD81 PE (Beckman Coulter), respectively, for 2 hours at room temperature as described previously (Tertel et al., 2020b, Chapter Four - Analysis of individual extracellular vesicles by imaging flow cytometry. In Methods in Enzymology, S. Spada, and L. Galluzzi, eds. (Academic Press), pp. 55-78). Accordingly, isotype controls were performed. For dye 2, final preparations were diluted to 500 μL for CD9 (end dilution factor of 1 to 100) and 200 μL for CD63 and CD81 analyses (end dilution factor of 1 to 40). The preparations for PKH67 were diluted to 100 μL for all three analyses (1:20 dilution).

### Detergent Control

To test for the EV nature of labelled objects detergent controls were performed by adding a sample volume of a 2% (w/v) NP-40 solution (Calbiochem, San Diego, CA, USA) to the samples.

### IFCM Analyses

All samples were measured using the built-in autosampler from U-bottom 96-well plates (Corning Falcon, cat 353077) with 5 min acquisition time per well on the AMNIS ImageStreamX Mark II Flow Cytometer (AMNIS/Luminex, Seattle, WA, USA). All data were acquired at 60× magnification at low flow rate (0.3795±0.0003 μL/min) and with removed beads option deactivated as described previously (Gorgens et al., 2019, Optimisation of imaging flow cytometry for the analysis of single extracellular vesicles by using fluorescence-tagged vesicles as biological reference material. J Extracell Vesicles 8, 1587567; Tertel et al., 2020a, High-Resolution Imaging Flow Cytometry Reveals Impact of Incubation Temperature on Labelling of Extracellular Vesicles with Antibodies. Cytometry A 97, 602-609). The data was analyzed as described previously (Tertel et al., 2020b).

Multi-Donor Mixed Lymphocyte Reaction (mdMLR)

The immunomodulatory potential of dye 2-labelled and non-labelled MSC-EV preparations were compared in a multi-donor mixed lymphocyte reaction assay (MLR) as described previously (Madel et al., 2020, Independent human mesenchymal stromal cell-derived extracellular vesicle preparations differentially affect symptoms in an advanced murine Graft-versus-Host-Disease model. bioRxiv, 2020.2012.2021.423658). Briefly, Ficoll prepared peripheral blood mononuclear cells (PBMC) of 12 healthy donors were mixed in equal proportions, aliquoted and stored in the vapour phase of liquid nitrogen until usage. After thawing 600,000 cells were seeded per well of a 96-well U-bottom shape plates (Corning, Kaiserslautern, Germany) and cultured in 10% human AB serum (produced in house) and 100 U/mL penicillin and 100 pg/mL streptomycin (Thermo Fisher Scientific) supplemented RPMI 1640 medium (Thermo Fisher Scientific) in a final volume of 200 μL per well, either in the presence or absence of MSC-EV preparations to be tested. After 5 days, cells were harvested, stained with a collection of different fluorescent labelled antibodies (CD4-BV785; BioLegend, San Diego, CA, USA; CD25-PE-Cy5.5; BD Bioscience; and CD54-AF700; EXBIO) and analysed on a Cytoflex flow cytometer (Software CytExpert 2.3, Beckman-Coulter). Activated and non-activated CD4⁺ T cells were discriminated by means of their CD25 and CD54 expression, respectively. Typically, 5 μL of MSC-EV preparations to be tested were applied into respective wells. The following antibodies were used to further discriminate subpopulations: CD8-BV650 (BioLegend), CD14-PO (EXBIO), CD19-ECD (Beckman Coulter) and CD56-APC (BioLegend). The evaluation of the data was carried out with the Kaluza software (Version 2.1, Beckman Coulter).

### Statistics

The statistics and graphical presentation were performed with GraphPad version 8.4.3. Mean values ±standard deviation are provided.

### Results

### CFSE, Calcein AM and BODIPY-TR-Ceramide do not Label MSC-EVs

The accuracy of conventionally used EV labelling dyes, specifically CFSE, Calcein AM, PKH67 and BODIPY-TR-Ceramide, and dye 2 of the invention were evaluated. MSC-EV preparations that have been extensively explored in various animal models had been obtained from supernatants of MSCs raised in 10% human platelet lysate supplemented media by well established PEG-ultracentrifugation protocols (Borger et al., 2020; Doeppner et al., 2015, Extracellular Vesicles Improve Post-Stroke Neuroregeneration and Prevent Postischemic Immunosuppression, Stem Cells Trans! Med 4, 1131-1143; Drommelschmidt et al., 2017, Mesenchymal stem cell-derived extracellular vesicles ameliorate inflammation-induced preterm brain injury. Brain Behav Immun 60, 220-232; Gussenhoven et al., 2019, Annexin Al as Neuroprotective Determinant for Blood-Brain Barrier Integrity in Neonatal Hypoxic-Ischemic Encephalopathy. J Clin Med 8; Kaminski et al., 2020, Mesenchymal Stromal Cell-Derived Extracellular Vesicles Reduce Neuroinflammation, Promote Neural Cell Proliferation and Improve Oligodendrocyte Maturation in Neonatal Hypoxic-Ischemic Brain Injury. Front Cell Neurosci 14, 601176; Kordelas et al., 2014; Ludwig et al., 2018; Ophelders et al., 2016, Mesenchymal Stromal Cell-Derived Extracellular Vesicles Protect the Fetal Brain After Hypoxia-lschemia. Stem Cells Trans! Med 5, 754-763; Wang et al., 2020, Mesenchymal Stromal Cell-Derived Small Extracellular Vesicles Induce Ischemic Neuroprotection by Modulating Leukocytes and Specifically Neutrophils. Stroke 51, 1825-1834). Since micelle formation of some of the conventional dyes have been reported and following the MIFlowCyt-EV recommendation (Welsh et al., 2020, MIFlowCyt-EV: a framework for standardized reporting of extracellular vesicle flow cytometry experiments, Journal of Extracellular Vesicles 9, 1713526), all of the labelling dyes but the EV sample were initially added to the NaCl-HEPES buffer, the buffer MSC-EVs are suspended in. Samples were processed according to the manufacturer's recommendation and analyzed by IFCM with protocols that we have successfully established for the characterization of antibody labelled MSC-EVs (Gorgens et al., 2019; Tertel et al., 2020a; Tertel et al., 2020b).

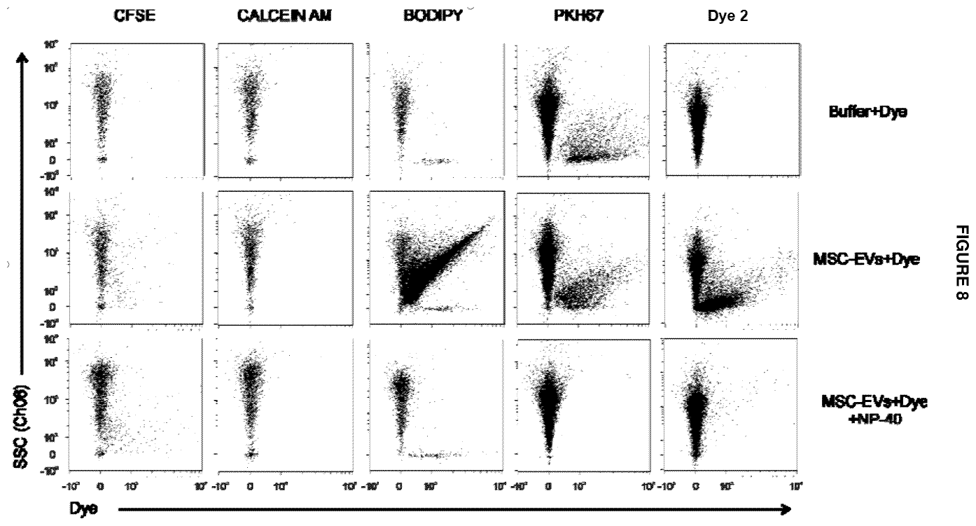

Depending on the manufacturer's protocol, different amounts of MSC-EV preparation was required. For the CFSE, Calcein-AM, BODIPY-TR Ceramide-(BODIPY) and dye 2 labelling volumes of 25 μL of MSC-EV preparation were used, and for PKH67 with 200 μL. Initially, all recorded 239 objects were analysed. Based on prior experience, sEVs appear as fluorescently labelled objects with minimal side scatter signals (SSC). Upon comparing the dye only solutions, CFSE, Calcein AM and dye 2 were observed to contain no objects. In contrast, upon analysing the BODIPY and PKH67 solutions, solid populations of labelled objects with minimal side scatter signals were identified (FIG. 8). This data implies micelle or aggregate formation of BODIPY and PKH dyes.

Subsequently, MSC-EV preparations labelled using the same procedure were analysed. In contrast to the buffer only solutions, solid populations of labelled objects were observed after BODIPY, PKH67 and dye 2 labelling and some objects following CFSE labelling (FIG. 8). Calcein AM failed to label any detectable objects. BODIPY+objects that were not recovered in the buffer-BODIPY solution control revealed side scatter signals that were much higher than those typically seen for small EVs (sEVs). In contrast, the light scattering properties of the objects specifically labelled with PKH67 or dye 2 reflect those of sEVs. In good agreement with published reports that PKH dyes increased the size of labelled EVs (Dehghani et al., 2020, Systematic Evaluation of PKH Labelling on Extracellular Vesicle Size by Nanoparticle Tracking Analysis, Scientific Reports 10, 9533; Morales-Kastresana et al., 2017, Labelling Extracellular Vesicles for Nanoscale Flow Cytometry. Sci Rep 7, 1878), the PKH67+objects specifically labelled in the MSC-EV preparation indicated higher side scatter signals than dye 2+objects (FIG. 8).

To determine whether the specifically labelled objects are detergent-sensitive, the dye labelled MSC-EV samples was treated with NP40. While the BODIPY+objects specifically detected in MSC-EV preparations, those with the higher light scattering properties, and all PKH67⁺ and dye 2⁺ objects disappeared following NP40 treatment. In contrast, the population of CFSE+objects and the BODIPY+objects with sEV light scattering properties were hardly affected by the NP40 treatment. To this end, it was considered neither detergent-resistant CFSE⁺ nor the BODIPY⁺ objects with low light scattering properties as small EVs, coupled to the failure of Calcein AM to label any specific objects, CFSE, Calcein AM and BODIPY were excluded from all later analyses and the accuracy of PKH67 and dye 2 as MSC-EV labelling dyes was focused on.

PKH67 Fails to Effectively Label CD9⁺, CD63⁺ and CD81⁺ sEVs

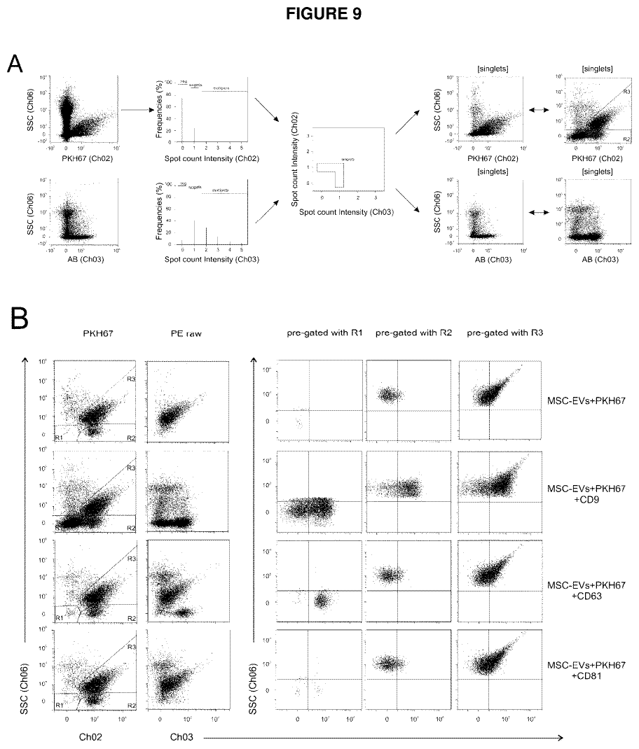

Next, the potential co-localisations of PKH67 with known EV markers was investigated. To this end, the well characterized MSC-EV preparations were used. These were stained either by PKH67 alone or in combination with any of the following antibodies: anti-CD9, anti-CD81 or anti-CD63 antibodies. To reduce the background noise and to exclude coincident events, the simultaneous detection of two or more independent objects at the same time (coincidences with high object numbers), an optimized gating strategy was applied. Briefly, objects recognised as singlets in the PKH67 channel without a simultaneous antibody signal or as singlets in the antibody channel without a simultaneous PKH67 signal, and events appearing in both channels as singlets not providing two individual objects, were focused on (FIG. 9A).

Upon plotting side scatter against PKH67 intensities, many more objects were recovered in samples that had been counterstained by anti-CD9 antibodies than in the PKH67 labelled buffer and MSC-EV containing controls. Most of these objects were negative for PKH67 and showed low SSC signals. The region containing these objects was defined as R1. For anti-CD9 staining, 6672±1170 objects were recovered in the region R1. Hardly any objects were recovered in R1 in the PKH67 labelled MSC-EV samples that were not counterstained by antibodies. A slight increase in objects numbers was recorded when PKH67 labelled MSC-EV samples were counterstained with anti-CD63 (221±42 objects) or anti-CD81 (96±32 objects) antibodies. Most of the objects that were positive for PKH67 revealed solid SSC signals. The region including these objects was defined as R3. A smaller number of PKH67 labelled objects was identified with low SSC signals that was clustered in a region defined as R2. In contrast to the number of objects in R1, the numbers of objects in R2 and R3 were only slightly affected by the antibody labelling procedures (FIGS. 9A and 9B). Within the antibody non-labelled control 2040±344 objects were recovered in R2, following anti-CD9 staining 2927±466 objects, following anti-CD63 staining 1747±141 objects and following anti-CD81 staining 1734±200 objects. In all antibody labelled MSC-EV preparations more objects were found in R3 (anti-CD9: 6625±803 objects; anti-CD63: 5915±271 objects; anti-CD81: 5777±675 objects) than in the antibody non-labelled control (2046±157 objects). To analyze objects within the 3 different regions in more detail, their antibody labelling intensities were plotted against PHK67 labelling intensities. The results clearly confirm that a huge proportion of the objects in R1 were effectively labelled by anti-CD9 antibodies. Although the R1 object populations were much smaller following anti-CD63 and anti-CD81 than after anti-CD9 antibody staining, a proportion of these objects was clearly recognized as CD63⁺ or CD81⁺, respectively (FIG. 9B). In contrast, all objects in R2 or in R3 appeared as CD63− and CD81− objects, most of which can be labelled by anti-CD9 antibodies. The frequencies of CD9+, CD63⁺ and CD81⁺ objects recovered in R1 are congruent to our previous observations that MSC-EV preparations contain a dominating CD9⁺CD81⁻ and a minor CD9⁺ CD81⁺ sEV population (Görgens et al., 2019). Overall, it was considered that most antibody stained objects in R1 were sEVs not being labelled by PKH67 and that most of the sEVs can only be detected if they are successfully stained with any of the 3 antibodies. Thus, this data questions the efficiency of PKH67 as a MSC-sEV labelling dye.

### Dye 2 Effectively Labels CD9+, CD63+ and CD81+ EVs in MSC-EV Preparations

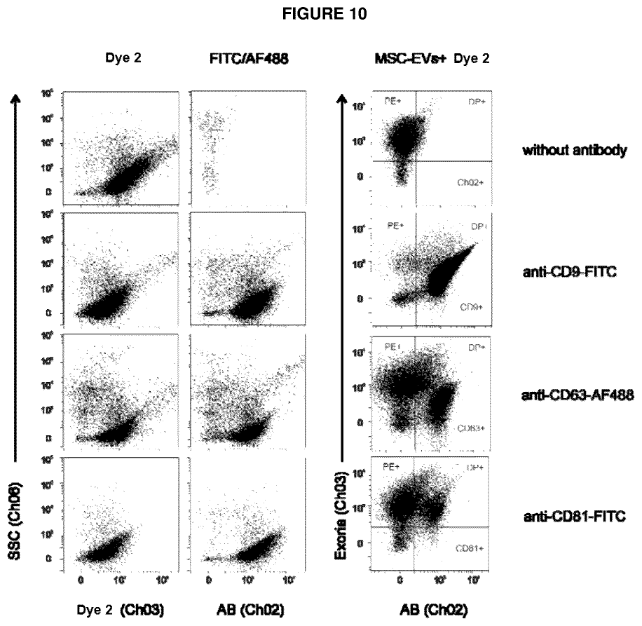

Next, the reliability of dye 2 of the invention as an EV labelling dye was investigated in a comparable manner to PKH67. To this end, the MSC-EV preparations (n=3) were either stained with dye 2 alone or in combination with anti-CD9, anti-CD63 or anti-CD81 antibodies, respectively. Without defining R1-3 sub gates, gating strategies were applied as depicted in FIG. 9A. In contrast to the PKH67 labelling experiments, many more objects with lower side scatter signal intensities were labelled by dye 2, even in the absence of any of the three different antibodies. No clear increase in the numbers of detected objects was observed between MSC-EV samples that were solely labelled by dye 2 or in addition by anti-CD9 antibodies (FIG. 10). Thus, in contrast to the PKH67 labelling, labelling with dye 2 is sufficient to label most of the sEVs within the MSC-EV preparations. All labelled objects were confirmed to be detergent sensitive. Overall the data demonstrate that dye 2 successfully labels most of the sEVs in the MSC-EV preparations.

Staining with Dye 2 does not Affect the Immunomodulatory Capacity of the MSC-EV Preparations

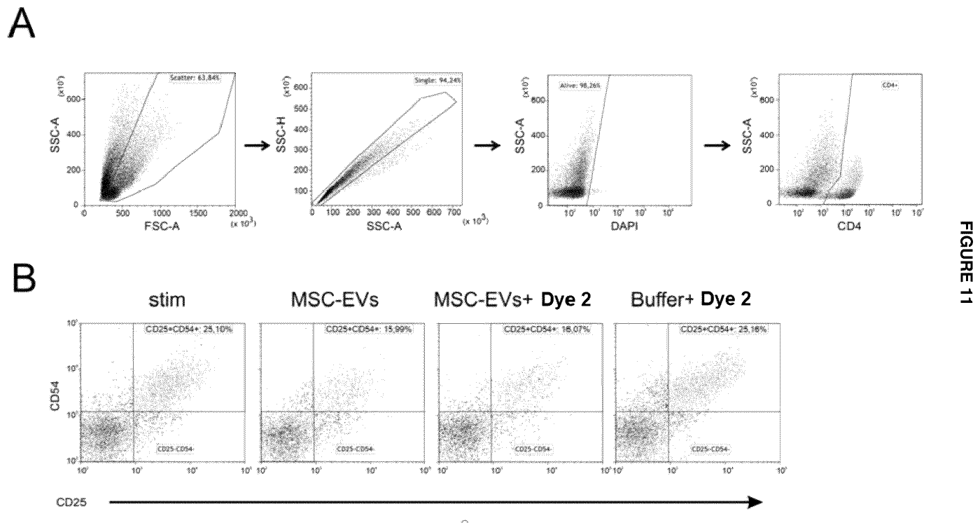

Next, it was investigated whether dye 2 of the invention affects the MSC-EV preparation's immunomodulatory capability. To this end, the activity of MSC-EV preparations stained with dye 2 were compared to corresponding, non-labelled MSC-EV preparations in a multi-donor mixed lymphocyte reaction (mdMLR) assay. Upon pooling of mononuclear cells from the peripheral blood of 12 different healthy donors (PBMCs), allogenic immune reactions are induced that can be monitored by the activation status of CD4⁺ T cells. Following 5 days in culture, approximately a quarter of all CD4⁺ T cells express the interleukin-2 receptor (CD25) and the intercellular adhesion molecule-1 (CD54), indicating T cell activation (FIG. 11). As previously described, MSC-EV preparations with immunomodulatory capabilities effectively reduce the content of activated CD4⁺ T cells (Madel et al., 2020). Consistently, in the presence of the non-labelled MSC-EV preparations only 16% of the monitored CD4⁺ T cells were found to display the activation cell surface markers (FIG. 11B). In the presence of dye 2-labelled MSC-EV preparations (n=3) we observed a comparable reduction in CD4⁺ T cell activation (FIG. 11B). Notably, dye 2 itself did not influence the activation status of CD4⁺ T cells (FIG. 11B). Thus, dye 2 does not recognizably affect the immunomodulatory capability of the applied MSC-EV preparations.

MSC-EVs Stained with Dye 2 Exhibit Different Uptake Potential Across Immune Cell Subtypes of a mdMLR Assay

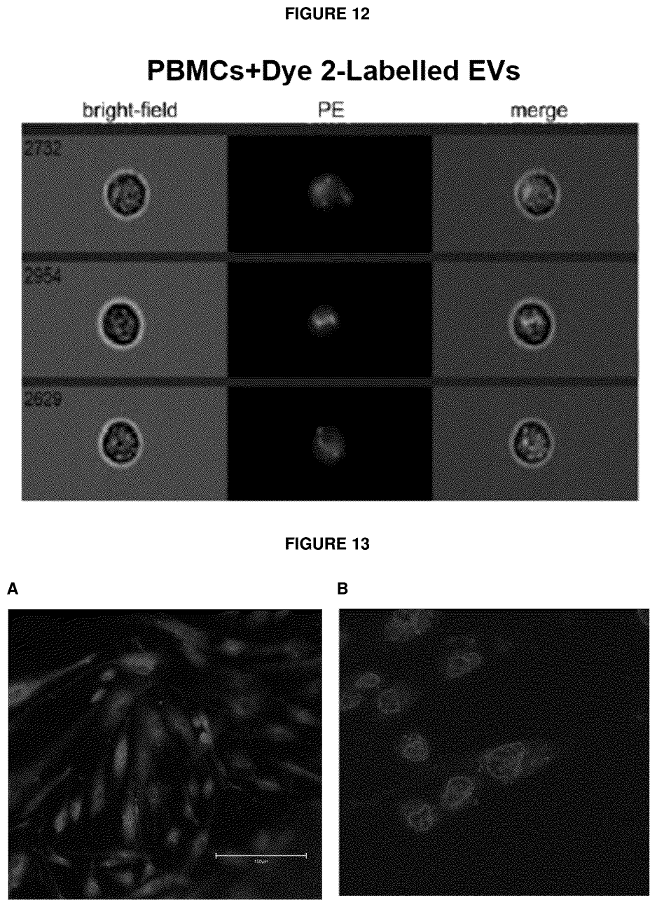

To investigate whether EV labelling with dye 2 of the invention allows the identification of EV-uptaking cells, the labelled EV uptake of the different immune cells within the mdMLR assay was examined. To this end, a pool of PBMCs derived from 12 healthy donors were cultured for 5 days in the presence of dye 2-labelled MSC-EVs (n=3) that had been cleared from excess dye 2 by ultrafiltration. Thereafter, cells were harvested, antibody labelled and analysed by flow cytometry. The content of dye 2-labelled cells within different PBMC subtypes was determined. Almost all monocytes (CD14⁺ cells, 99%) revealed dye 2 signals. In contrast, only proportions of the different lymphocytes appeared as dye 2 positive cells, i.e. 71% of all CD4⁺ T cells (CD4⁺ cells), 34% of all CD8⁺ T cells (CD8⁺ cells), 72% of all B cells (CD19⁺ cells) and 15% of all NK cells (CD56⁺ cells). In addition, IFCM was used to visualise the subcellular staining of dye 2 positive cells (FIG. 12). Obtained images reveal concrete labelled structures that according to our experience are located subcellularly. Thus, EVs labelled with dye 2 within the MSC-EV preparations were specifically taken up by different contents of the immune cell types within the assay.

### Example 12. Visualisation of Extracellular Vesicles Labelled with Dye

Electron microscopy techniques can be applied to visualise purified EVs. Further, confocal microscopy can be used to track the movement of labelled EVs inside cells during in vitro and other assays. FIG. 13 illustrates EVs stained with dye 2 of the invention in NHDFs viewed by conventional (FIG. 13A) and confocal (FIG. 13B) microscopy. As described herein, dye 2 can also be used in EV flow cytometry.
An extracellular vesicle (EV) labelled with a fluorescent dye of formula (I):

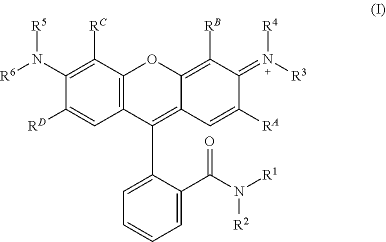

wherein
R¹ and R² are each independently selected from optionally substituted C₁₋₂₆aliphatic, optionally substituted C₁₋₂₆heteroaliphatic, optionally substituted C₁₋₂₆aliphatic-OH, optionally substituted C₁₋₂₆heteroaliphatic-OH and OH, wherein at least one of R¹ and R² is selected from optionally substituted C₁₃₋₂₆aliphatic or optionally substituted C₁₃₋₂₆heteroaliphatic;
R³, R⁴, R⁵ and R⁶ are independently selected from optionally substituted C₁₋₆alkyl, optionally substituted C₁₋₆haloalkyl, optionally substituted C₁₋₆heteroalkyl, optionally substituted aryl and optionally substituted C₁₋₆alkylaryl or
R³ and R⁴ together with the nitrogen atom to which they are attached form an optionally substituted 3-7 membered heterocyclic ring; and/or
R⁵ and R⁶ together with the nitrogen atom to which they are attached form an optionally substituted 3-7 membered heterocyclic ring; and
R^{A}, R^{B}, R^{C} and R^{D} are independently selected from hydrogen, fluorine and chlorine.

2. The EV of claim 1, wherein
R¹ is selected from C₁₃₋₂₆alkyl, C₁₃₋₂₆alkenyl and C₁₃₋₂₆heteroalkyl;
R² is selected from C₁₋₆alkyl, C₁₋₆heteroalkyl, C₁₋₆alkyl-OH and C₁₋₆heteroalkyl-OH; and
R³, R⁴, R⁵ and R⁶ are each independently C₁₋₆alkyl or C₁₋₆haloalkyl.

3. The EV of claim 1 or claim 2, wherein R¹ is selected from C₁₃₋₂₆alkyl, C₁₃₋₂₆alkenyl, C₂alkyl-O-C₁₀₋₂₆alkyl and OC₁₂₋₂₅alkyl.

4. The EV of any one of claims 1-3, wherein R¹ is selected from C₁₄₋₂₂alkyl and C₁₄₋₂₂alkenyl.

5. The EV of any one of claims 1-4, wherein R² is selected from C₁₋₆alkyl and C₁₋₆alkylOR⁸, wherein R⁸ is selected from hydrogen and methyl.

6. The EV of any one of claims 1-5, wherein R³, R⁴, R⁵ and R⁶ are each independently C₁₋₆alkyl or C₁₋₆haloalkyl.

7. The EV of any one of claims 1-6, wherein R³, R⁴, R⁵ and R⁶ are each independently C₁₋₃alkyl or C₁₋₃fluoroalkyl.

8. The EV of any one of claims 1-7, wherein R^{A}, R^{B}, R^{C} and R^{D} are each hydrogen.

9. The EV of any one of claims 1-8, wherein the EV is selected from an exosome, an exomere, a microvesicle, an oncosome and an apoptotic body.

10. A salt of a compound of formula (II): 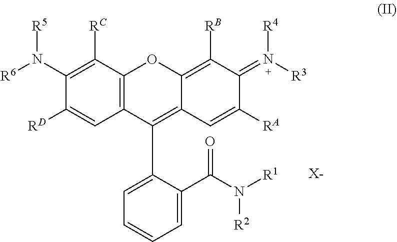
wherein
R¹ is selected from optionally substituted C₁₃₋₂₆aliphatic, C₁₃₋₂₆heteroaliphatic comprising 1 or 2 heteroatoms in the longest linear heteroaliphatic chain, optionally substituted C₁₃₋₂₆aliphatic-OH, optionally substituted C₁₃₋₂₆heteroaliphatic-OH and OH;
R² is selected from optionally substituted C₁₋₆aliphatic, optionally substituted C₁₋₆heteroaliphatic, optionally substituted C₁₋₆aliphatic-OH, optionally substituted C₁₋₆heteroaliphatic-OH, and OH;
R³, R⁴, R⁵ and R⁶ are independently selected from optionally substituted C₁₋₆alkyl, optionally substituted C₁₋₆haloalkyl, optionally substituted aryl and optionally substituted C₁₋₆alkylaryl or
R³ and R⁴ together with the nitrogen atom to which they are attached form an optionally substituted 3-7 membered heterocyclic ring; and/or
R³ and R⁴ together with the nitrogen atom to which they are attached form an optionally substituted 3-7 membered heterocyclic ring; and
R^{A}, R^{B}, R^{C} and R^{D} are independently selected from a hydrogen, fluorine and chlorine; and
X⁻ is selected from fluoride, chloride, bromide, iodide, acetate, trifluoroacetate, benzoate, C₁₋₂₆alkyl sulfonate, arylsulfonate, nitrate, hexafluorophosphate, tetrafluoroborate, trichlorozincate(II), tetrachloroferrate and perchlorate.

11. The salt of claim 10, wherein R¹ is selected from C₁₃₋₂₆alkyl, C₁₃₋₂₆alkenyl and C₁₃₋₂₆heteroalkyl comprising 1 or 2 heteroatoms in the longest linear heteroalkyl chain;
R² is selected from C₁₋₆alkyl, C₁₋₆heteroalkyl, C₁₋₆alkyl-OH and C₁₋₆heteroalkyl-OH; and
R³, R⁴, R⁵ and R⁶ are each independently C₁₋₆alkyl or C₁₋₆haloalkyl.

12. The salt of claim 10 or claim 11, wherein R¹ is selected from C₁₃₋₂₆alkyl, C₁₃₋₂₆alkenyl, C₂alkyl-O-CC₁₀₋₂₆alkyl and OCC₁₂₋₂₅alkyl.

13. The salt of any one of claims 10-12, wherein R¹ is selected from C₁₄₋₂₂alkyl and C₁₄₋₂₂alkenyl.

14. The salt of any one of claims 10-13, wherein R² is selected from C₁₋₆alkyl and C₁₋₆alkylOR⁸, wherein R⁸ is selected from hydrogen and methyl.

15. The salt of any one of claims 10-14, wherein R³, R⁴, R⁵ and R⁶ are each independently C₁₋₆alkyl or C₁₋₆haloalkyl.

16. The salt of any one of claims 10-15, wherein R³, R⁴, R⁵ and R⁶ are each independently C₁₋₃alkyl or C₁₋₃fluoroalkyl.

17. The salt of any one of claims 10-16, wherein R^{A}, R^{B}, R^{C} and R^{D} are each hydrogen.

18. The salt of any one of claims 10-17 having a structure selected from any one of the following: 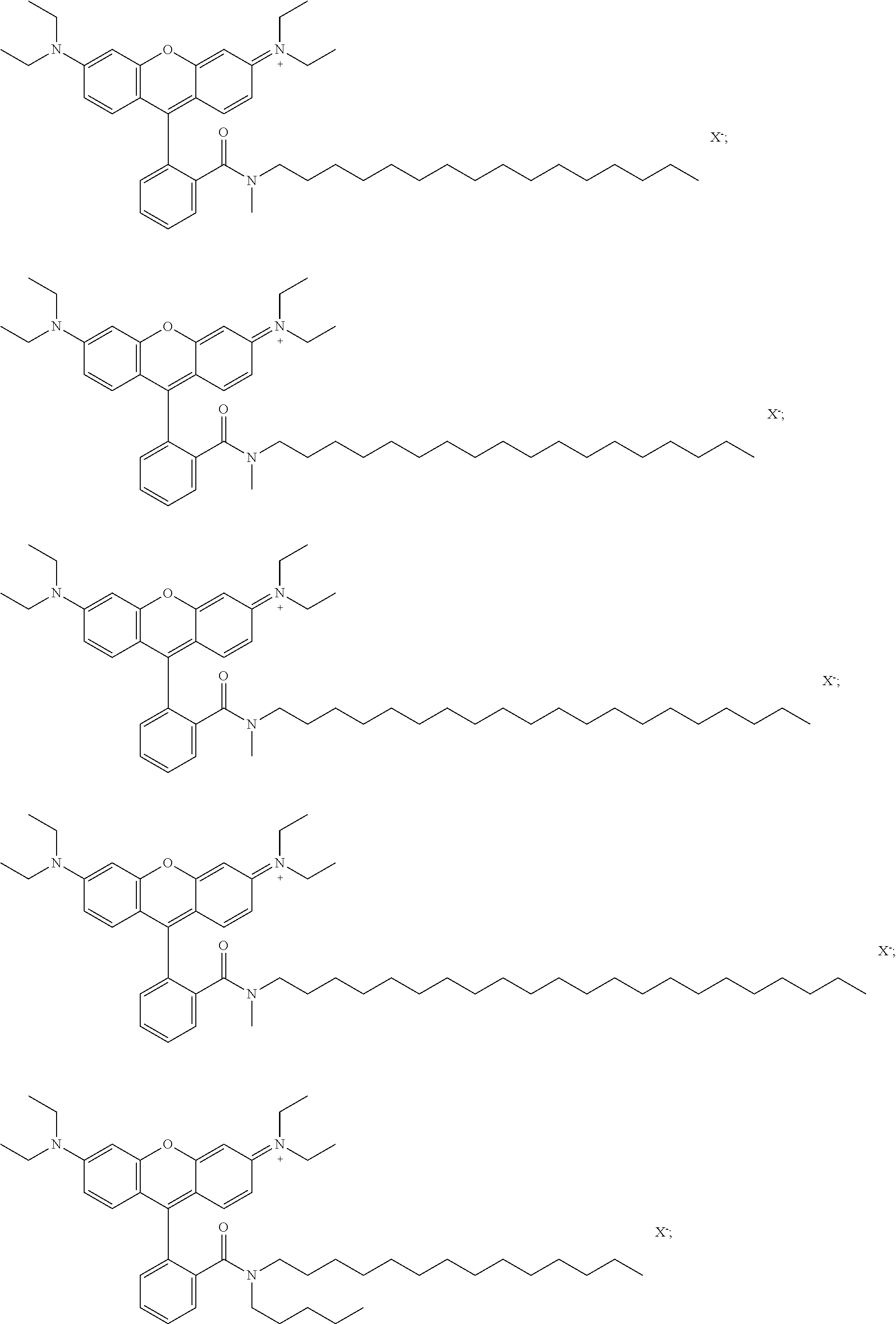 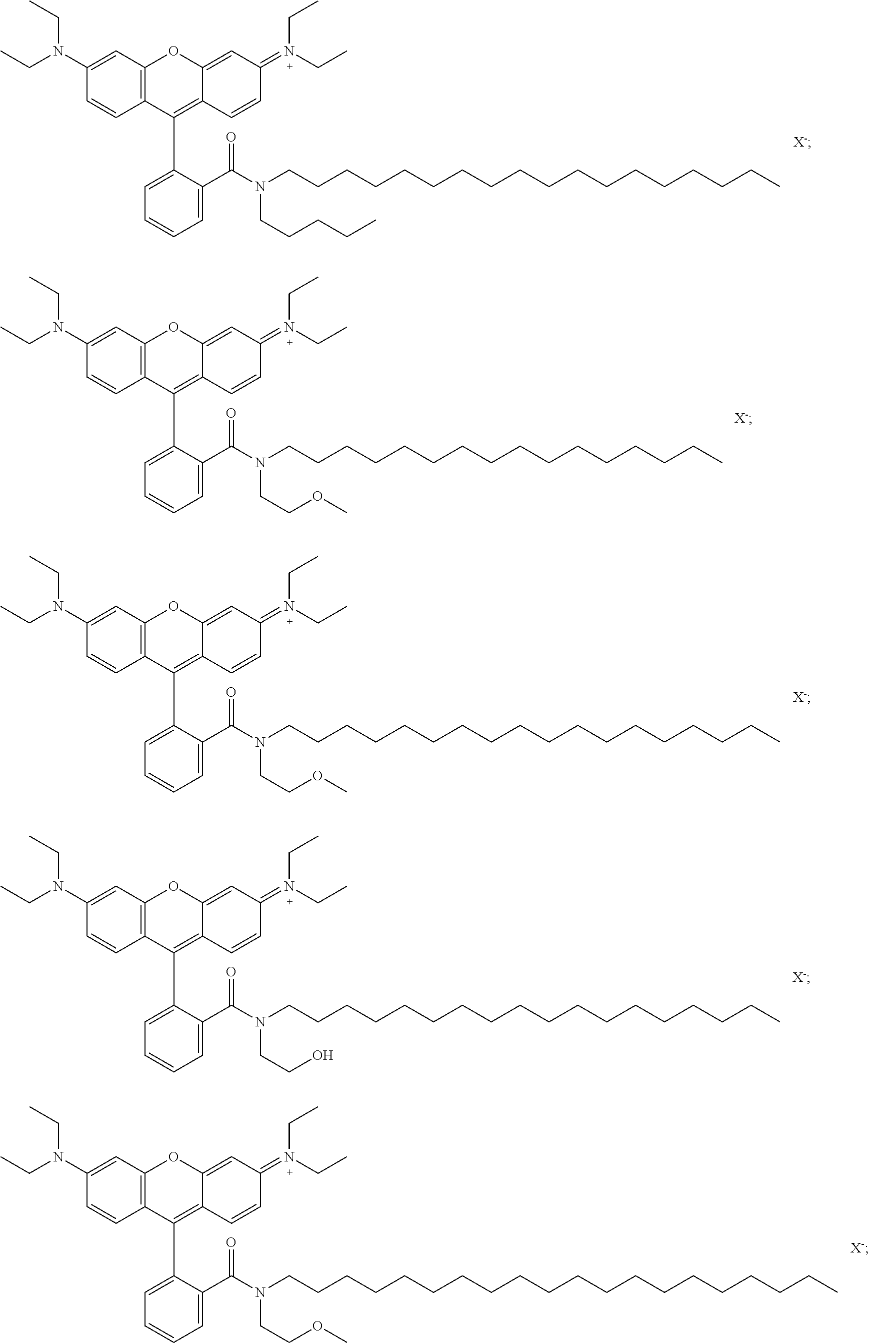 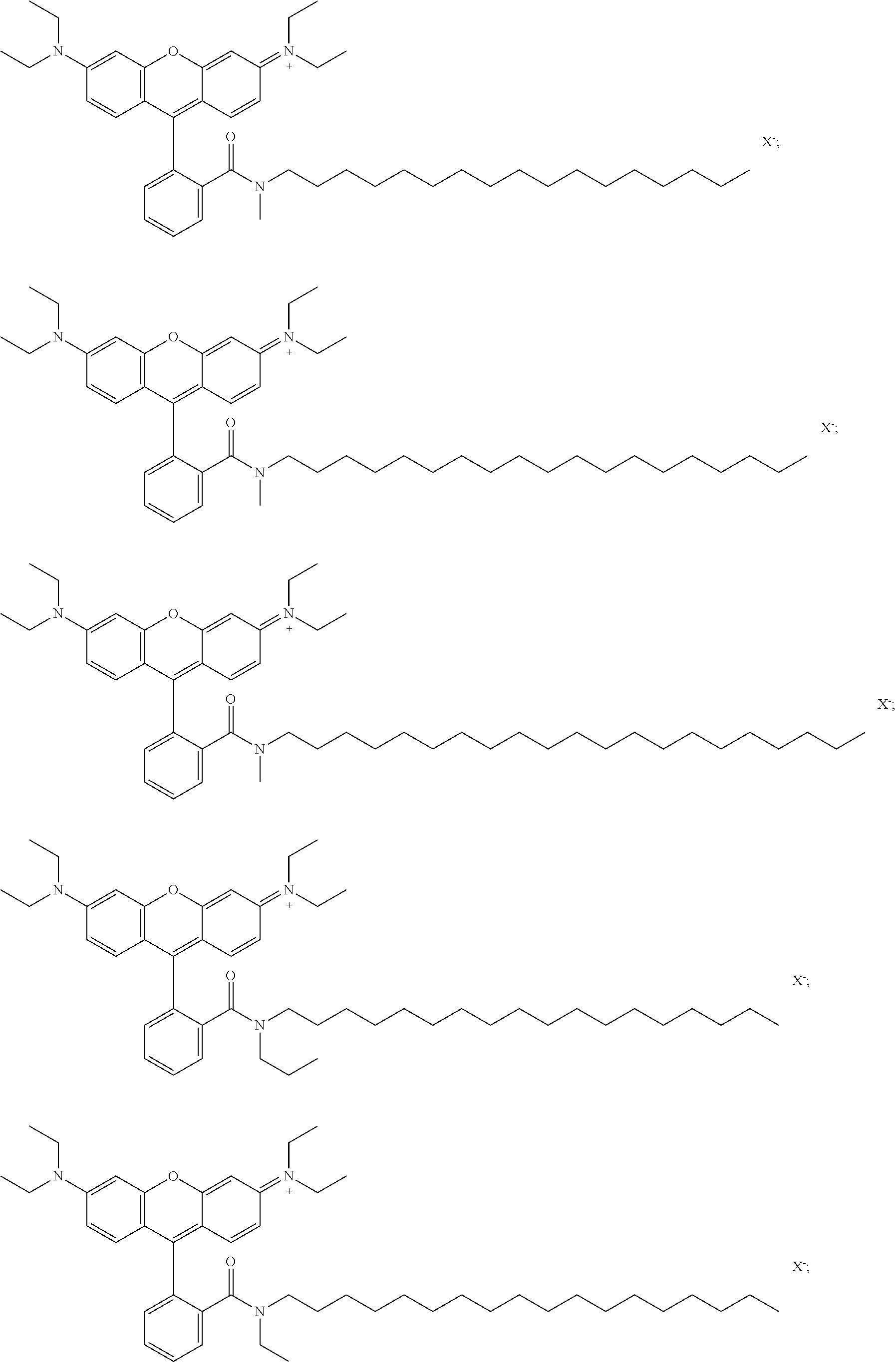 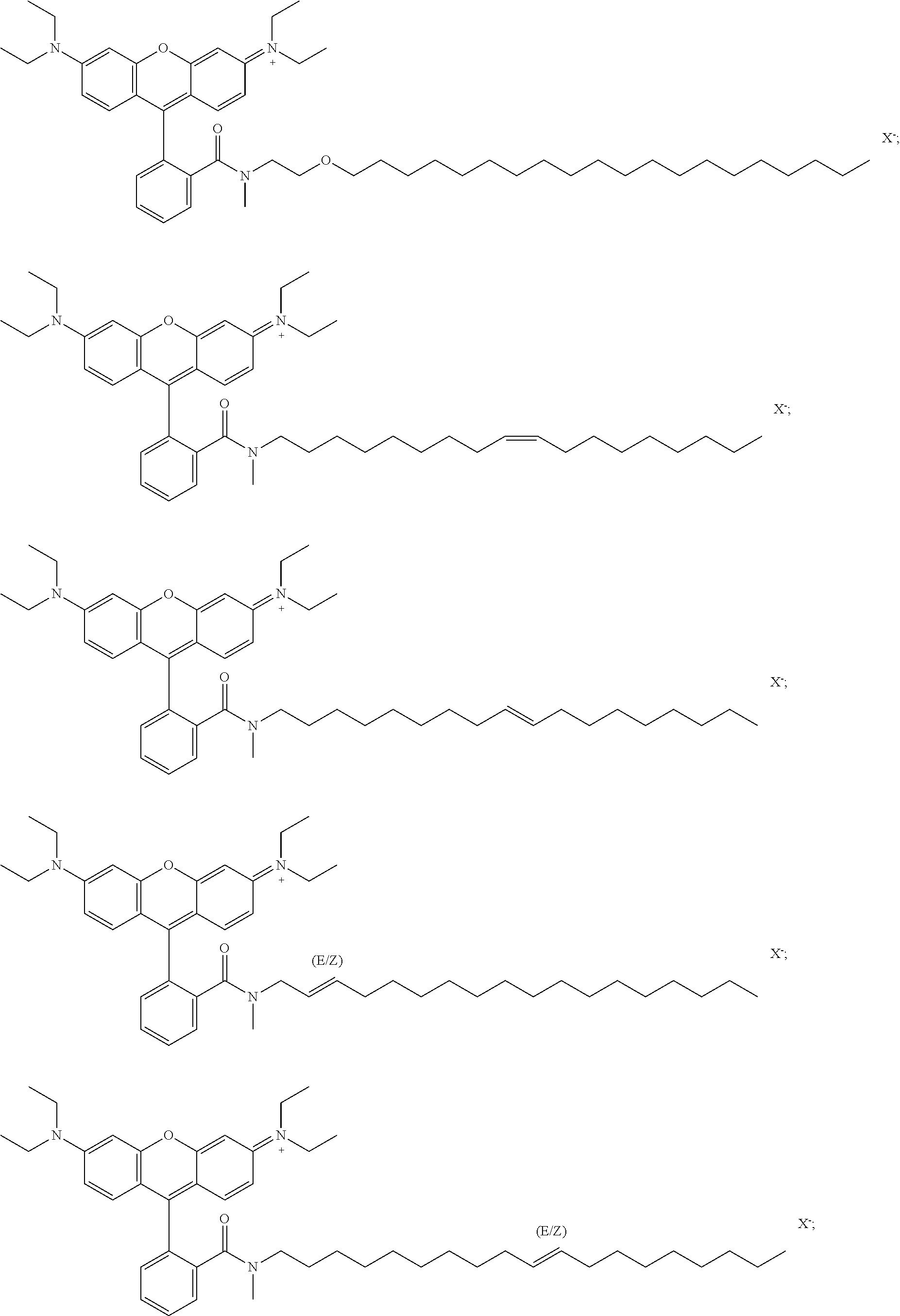 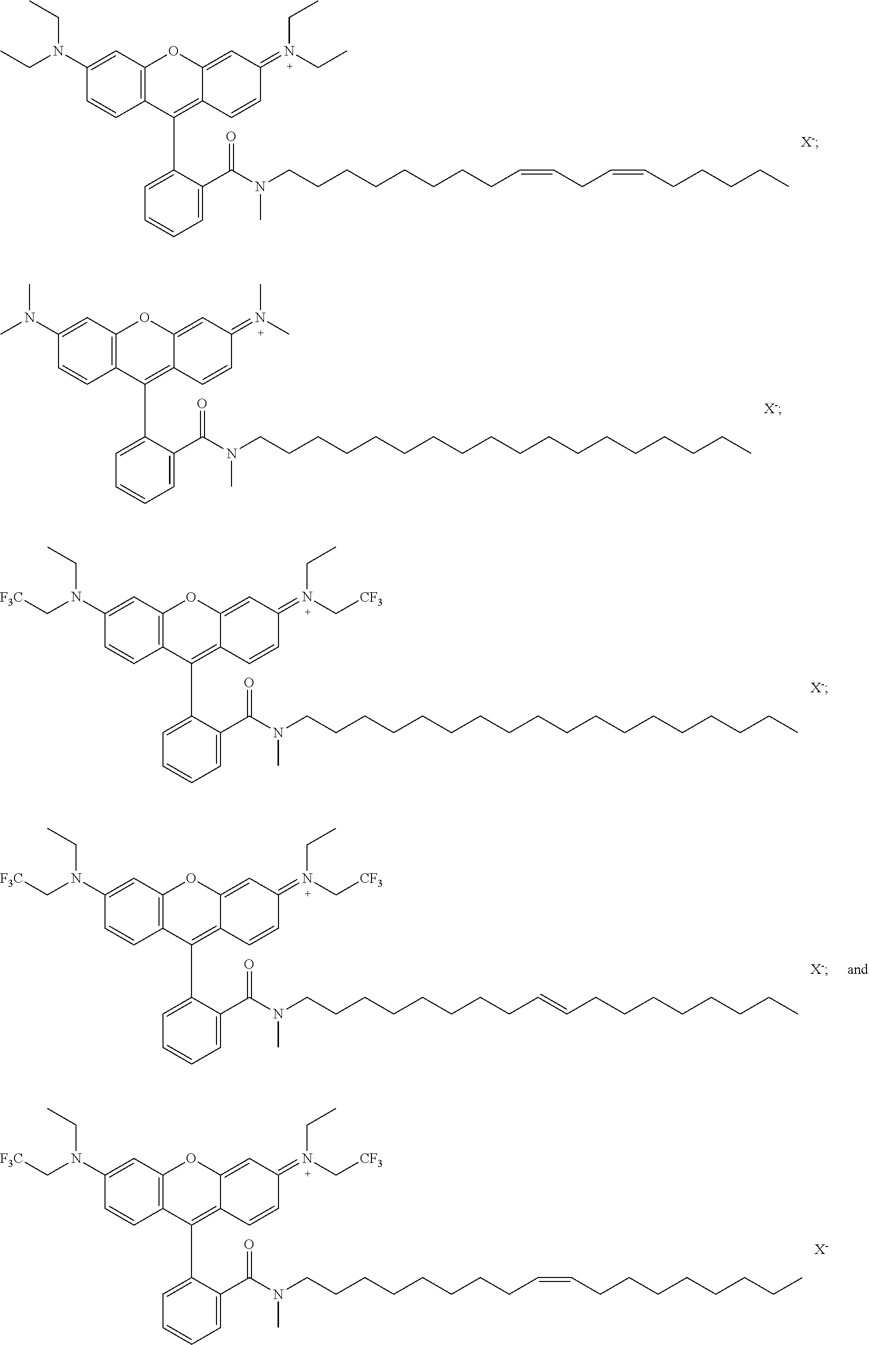
wherein X⁻ is selected from chloride, bromide, iodide, acetate, trifluoroacetate, nitrate, hexafluorophosphate, tetrafluoroborate and perchlorate.

19. The salt of any one of claims 10-18 wherein X⁻ is selected from chloride, bromide and iodide.

20. The salt of any one of claims 10-19 wherein X⁻ is chloride.

21. The salt of claim 10 selected from any one of the following:

| | Structure |
|---|---|
| 1 | 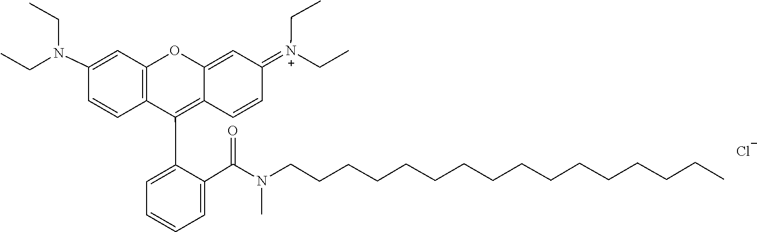 [SMILES: CCCCCCCCCCCCCCCCN(C)C(=O)c1ccccc1-c1c2ccc(=[N+](CC)CC)cc-2oc2cc(N(CC)CC)ccc12 ; [Cl-]] |
| 2 | 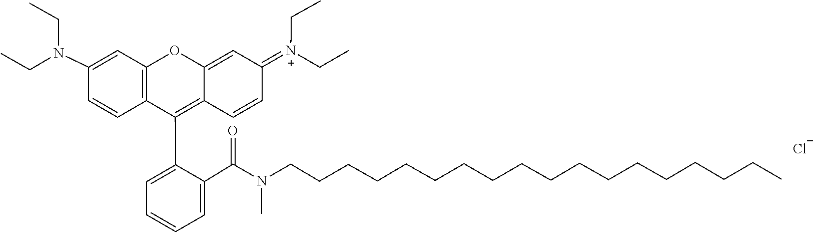 [SMILES: CCCCCCCCCCCCCCCCCCN(C)C(=O)c1ccccc1-c1c2ccc(=[N+](CC)CC)cc-2oc2cc(N(CC)CC)ccc12 ; [Cl-]] |
| 3 | 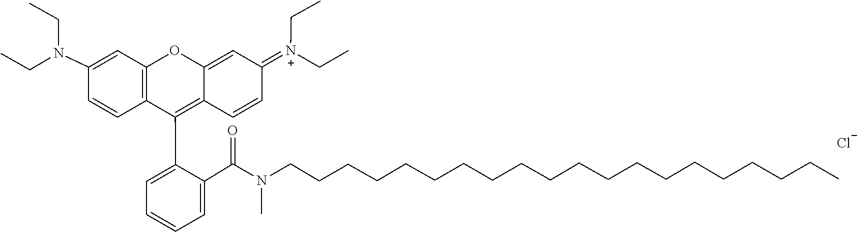 [SMILES: CCCCCCCCCCCCCCCCCCCCN(C)C(=O)c1ccccc1-c1c2ccc(=[N+](CC)CC)cc-2oc2cc(N(CC)CC)ccc12 ; [Cl-]] |
| 4 | 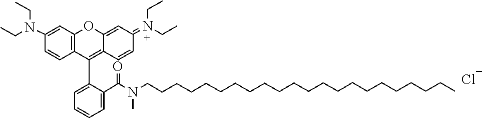 [SMILES: CCCCCCCCCCCCCCCCCCCCCCN(C)C(=O)c1ccccc1-c1c2ccc(=[N+](CC)CC)cc-2oc2cc(N(CC)CC)ccc12 ; [Cl-]] |
| 5 | 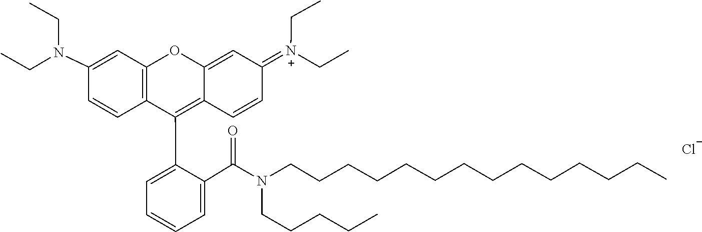 [SMILES: CCCCCCCCCCCCCCN(CCCCC)C(=O)c1ccccc1-c1c2ccc(=[N+](CC)CC)cc-2oc2cc(N(CC)CC)ccc12 ; [Cl-]] |
| 6 | 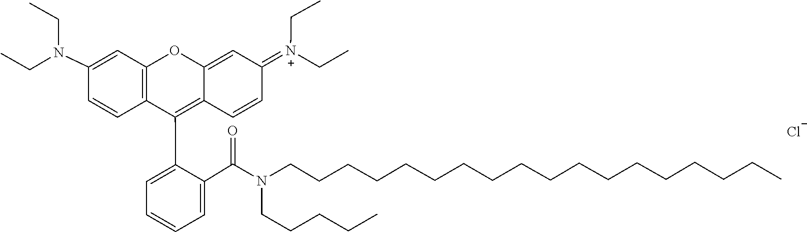 [SMILES: CCCCCCCCCCCCCCCCCCN(CCCCC)C(=O)c1ccccc1-c1c2ccc(=[N+](CC)CC)cc-2oc2cc(N(CC)CC)ccc12 ; [Cl-]] |
| 7 | 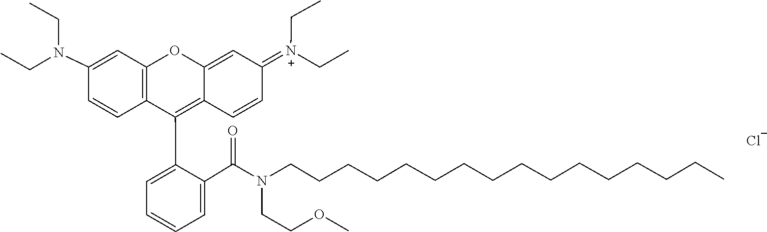 [SMILES: CCCCCCCCCCCCCCCCN(CCOC)C(=O)c1ccccc1-c1c2ccc(=[N+](CC)CC)cc-2oc2cc(N(CC)CC)ccc12 ; [Cl-]] |
| 8 | 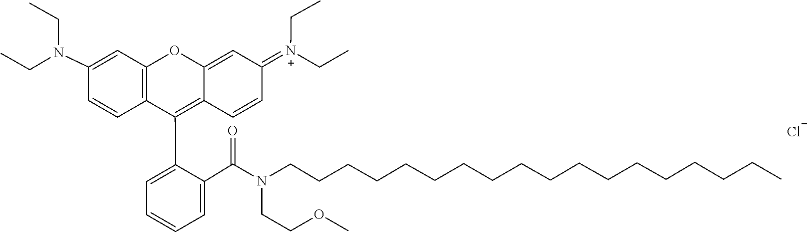 [SMILES: CCCCCCCCCCCCCCCCCCN(CCOC)C(=O)c1ccccc1-c1c2ccc(=[N+](CC)CC)cc-2oc2cc(N(CC)CC)ccc12 ; [Cl-]] |
| 9 | 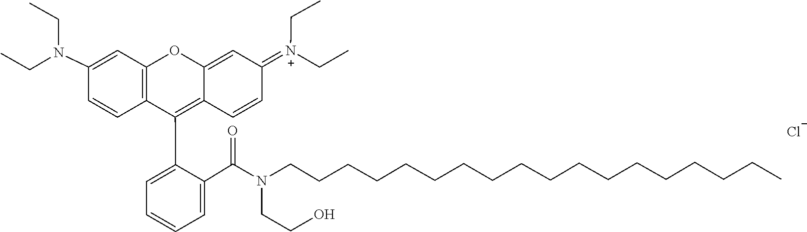 [SMILES: CCCCCCCCCCCCCCCCCCN(CCO)C(=O)c1ccccc1-c1c2ccc(=[N+](CC)CC)cc-2oc2cc(N(CC)CC)ccc12 ; [Cl-]] |
| 10 | 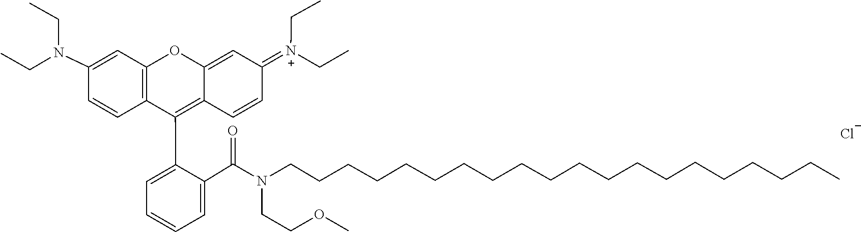 [SMILES: CCCCCCCCCCCCCCCCCCCCN(CCOC)C(=O)c1ccccc1-c1c2ccc(=[N+](CC)CC)cc-2oc2cc(N(CC)CC)ccc12 ; [Cl-]] |
| 11 | 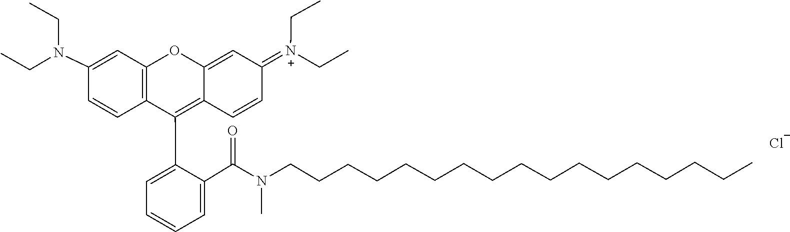 [SMILES: CCCCCCCCCCCCCCCCCN(C)C(=O)c1ccccc1-c1c2ccc(=[N+](CC)CC)cc-2oc2cc(N(CC)CC)ccc12 ; [Cl-]] |
| 12 | 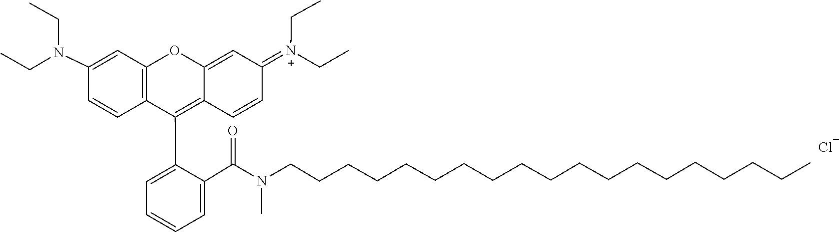 [SMILES: CCCCCCCCCCCCCCCCCCCN(C)C(=O)c1ccccc1-c1c2ccc(=[N+](CC)CC)cc-2oc2cc(N(CC)CC)ccc12 ; [Cl-]] |
| 13 | 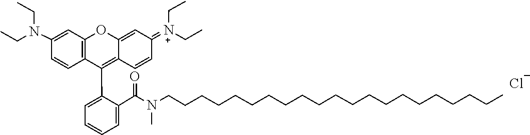 [SMILES: CCCCCCCCCCCCCCCCCCCCCN(C)C(=O)c1ccccc1-c1c2ccc(=[N+](CC)CC)cc-2oc2cc(N(CC)CC)ccc12 ; [Cl-]] |
| 14 | 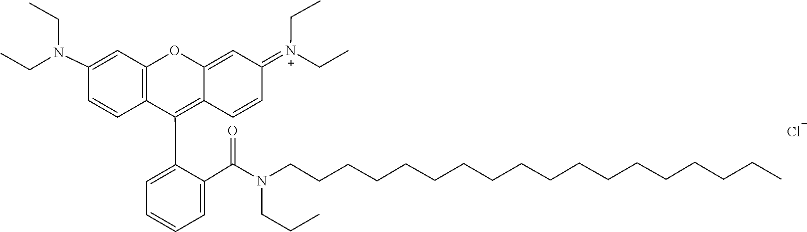 [SMILES: CCCCCCCCCCCCCCCCCCN(CCC)C(=O)c1ccccc1-c1c2ccc(=[N+](CC)CC)cc-2oc2cc(N(CC)CC)ccc12 ; [Cl-]] |
| 15 | 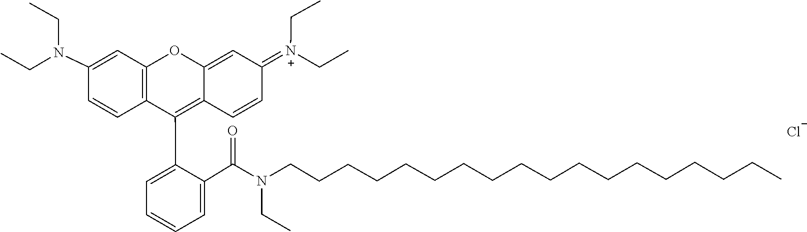 [SMILES: CCCCCCCCCCCCCCCCCCN(CC)C(=O)c1ccccc1-c1c2ccc(=[N+](CC)CC)cc-2oc2cc(N(CC)CC)ccc12 ; [Cl-]] |
| 16 | 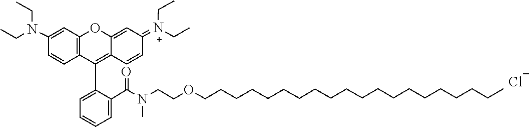 [SMILES: CCCCCCCCCCCCCCCCCCCCOCCN(C)C(=O)c1ccccc1-c1c2ccc(=[N+](CC)CC)cc-2oc2cc(N(CC)CC)ccc12 ; [Cl-]] |
| 17 | 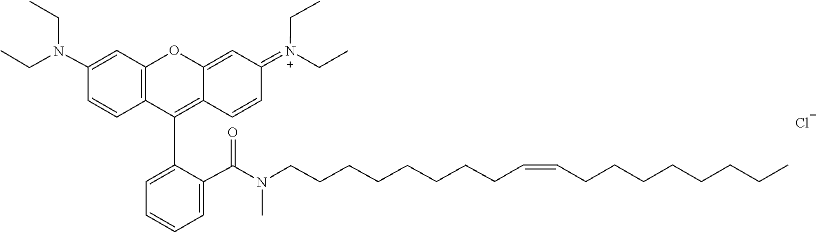 [SMILES: CCCCCCCC/C=C\CCCCCCCCN(C)C(=O)c1ccccc1-c1c2ccc(=[N+](CC)CC)cc-2oc2cc(N(CC)CC)ccc12 ; [Cl-]] |
| 18 | 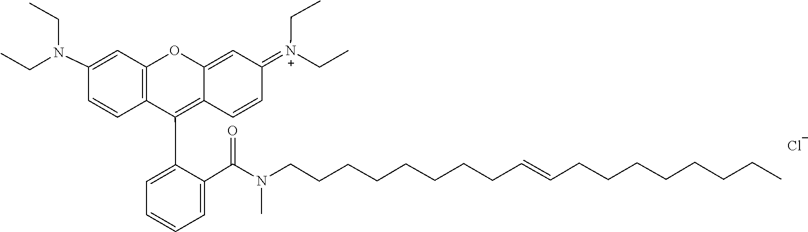 [SMILES: CCCCCCCC/C=C/CCCCCCCCN(C)C(=O)c1ccccc1-c1c2ccc(=[N+](CC)CC)cc-2oc2cc(N(CC)CC)ccc12 ; [Cl-]] |
| 19 | 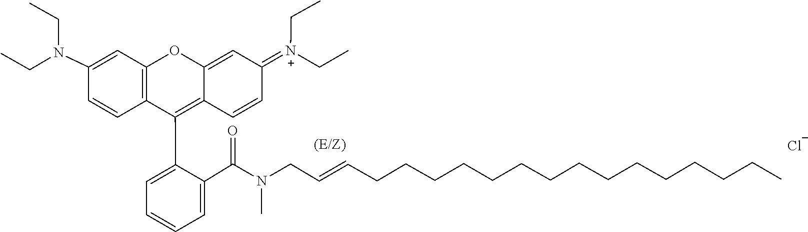 [SMILES: CCCCCCCCCCCCCCC/C=C/CN(C)C(=O)c1ccccc1-c1c2ccc(=[N+](CC)CC)cc-2oc2cc(N(CC)CC)ccc12 ; [Cl-]] |
| 20 | 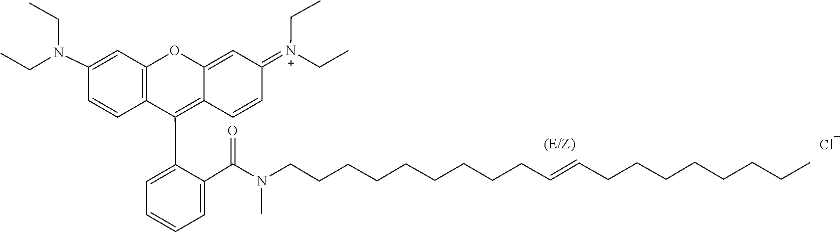 [SMILES: CCCCCCCC/C=C/CCCCCCCCCN(C)C(=O)c1ccccc1-c1c2ccc(=[N+](CC)CC)cc-2oc2cc(N(CC)CC)ccc12 ; [Cl-]] |
| 21 | 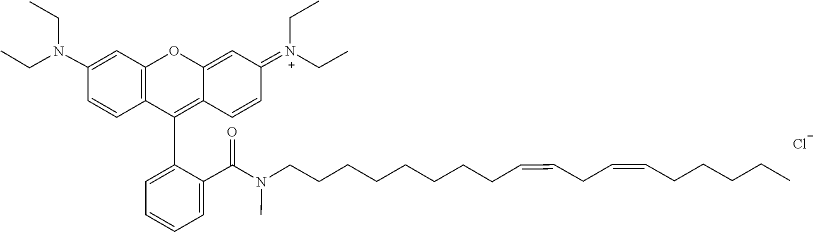 [SMILES: CCCCC/C=C\C/C=C\CCCCCCCCN(C)C(=O)c1ccccc1-c1c2ccc(=[N+](CC)CC)cc-2oc2cc(N(CC)CC)ccc12 ; [Cl-]] |
| 22 | 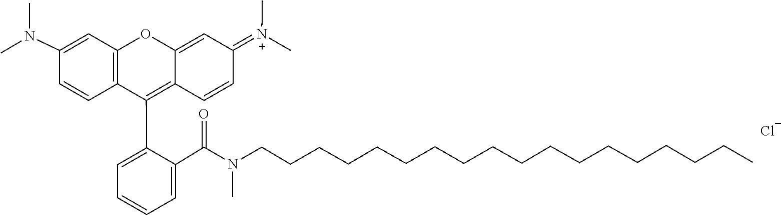 [SMILES: CCCCCCCCCCCCCCCCCCN(C)C(=O)c1ccccc1-c1c2ccc(=[N+](C)C)cc-2oc2cc(N(C)C)ccc12 ; [Cl-]] |
| 23 | 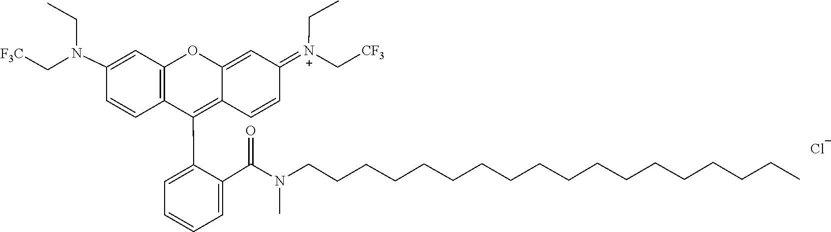 [SMILES: CCCCCCCCCCCCCCCCCCN(C)C(=O)c1ccccc1-c1c2cc/c(=[N+](/CC)CC(F)(F)F)cc-2oc2cc(N(CC)CC(F)(F)F)ccc12 ; [Cl-]] |
| 24 | 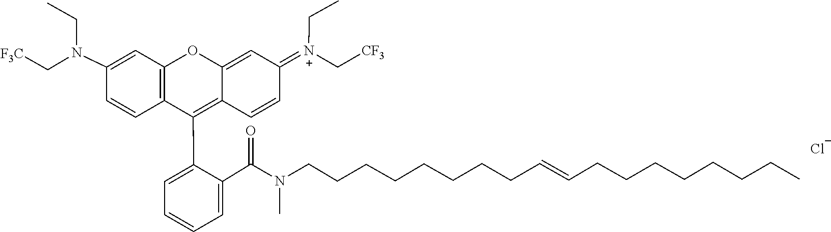 [SMILES: CCCCCCCC/C=C/CCCCCCCCN(C)C(=O)c1ccccc1-c1c2cc/c(=[N+](/CC)CC(F)(F)F)cc-2oc2cc(N(CC)CC(F)(F)F)ccc12 ; [Cl-]] |
| 25 | 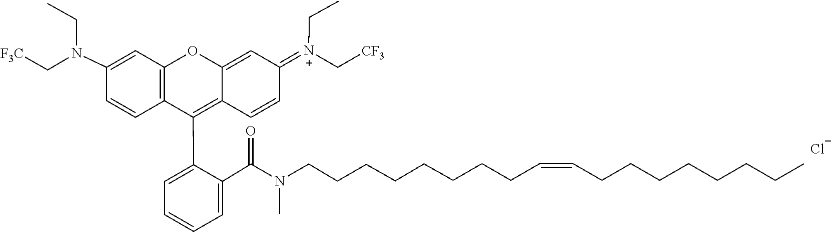 [SMILES: CCCCCCCC/C=C\CCCCCCCCN(C)C(=O)c1ccccc1-c1c2cc/c(=[N+](/CC)CC(F)(F)F)cc-2oc2cc(N(CC)CC(F)(F)F)ccc12 ; [Cl-]] |

22. A method of labelling an extracellular vesicle (EV) or a cell, the method comprising combining the salt of any one of claims 10-21 with the EV or the cell.

23. A method of preparing a labelled extracellular vesicle (EV), the method comprising combining the salt of any one of claims 10-21 with the EV to thereby label the EV with the compound of formula (I).

24. The method of claim 22 or claim 23 wherein the combining step comprises adding the salt to an aqueous solution comprising the EV or cell.

25. The method of claim 23 wherein the aqueous solution comprises a buffer.

26. A method of detecting an extracellular vesicle (EV) or a cell, the method comprising:
exposing an EV or cell labelled with a fluorescent dye of formula (I) as defined in any one of claims 1-9 to a source of light selected to excite the fluorescent dye of formula (I); and
detecting fluorescence emission from the fluorescent dye.

27. A method of tracking an extracellular vesicle (EV) or a cell, the method comprising:
exposing an EV or cell labelled with a fluorescent dye of formula (I) as defined in any one of claims 1-9 to a source of light selected to excite the fluorescent dye of formula (I); and
detecting fluorescence emission from the fluorescent dye.

28. Use of the salt of any one of claims 10-21 for labelling an EV or a cell.

29. A kit for fluorescently labelling an EV or a cell, or when used for fluorescently labelling an EV or a cell, the kit comprising:
a salt according to any one of claims 10-21; and optionally
instructions for its use to fluorescently label an extracellular vesicle or cell.

30. A method for delivering a therapeutic cargo to a cell, the method comprising contacting a cell with an EV of any one of claims 1-9, wherein the EV comprises a therapeutic cargo.
